(19) United States
(12) Patent Application Publication
(10) Pub. No.: US 2024/0033343 A1
(43) Pub. Date: Feb. 1, 2024

(54) MODIFIED POLYPEPTIDES WITH IMPROVED PROPERTIES

(71) Applicant: THE UNIVERSITY OF QUEENSLAND, St. Lucia (AU)

(72) Inventors: Keith CHAPPELL, Kenmore (AU); Paul YOUNG, Ashgrove (AU); Daniel WATTERSON, Dutton Park (AU)

(73) Assignee: THE UNIVERSITY OF QUEENSLAND, St. Lucia (AU)

(21) Appl. No.: 18/023,136

(22) PCT Filed: Aug. 26, 2021
(86) PCT No.: PCT/IB2021/057819

§ 371 Date: Feb. 24, 2023

(30) Foreign Application Priority Data
Aug. 26, 2020 (AU) 2020903050

(51) Int. Cl. A61K 39/215; C07K 14/005; A61P 31/14

(52) U.S. Cl. CPC A61K 39/215; C07K 14/005; A61P 31/14; C12N 2770/20034; C12N 2770/20022; C07K 2319/73; C07K 2319/91; A61K 2039/575

(57) ABSTRACT

This disclosure relates generally to modified SARS-CoV-2 spike polypeptides. More particularly, the present disclosure relates to modified SARS-CoV-2 spike proteins with improved properties, to chimeric polypeptides comprising these modified proteins, and to complexes comprising the chimeric polypeptides. The present disclosure also relates to the use of these modified polypeptides, chimeric polypeptides and complexes in compositions and methods for eliciting an immune response to ACE2-interacting coronaviruses, including SARS-CoV-2, and/or for treating or inhibiting the development of ACE2-interacting coronaviruses infections.

### FIELD

This disclosure relates generally to modified SARS-CoV-2 spike polypeptides. More particularly, the present disclosure relates to modified SARS-CoV-2 spike proteins with improved properties, to chimeric polypeptides comprising these modified proteins, and to complexes comprising the chimeric polypeptides. The present disclosure also relates to the use of these modified polypeptides, chimeric polypeptides and complexes in compositions and methods for eliciting an immune response to ACE2-interacting coronaviruses, including SARS-CoV-2, and/or for treating or inhibiting the development of ACE2-interacting coronaviruses infections.

### BACKGROUND

Coronaviruses are a group of related viruses that cause diseases in humans and animals. In humans, coronaviruses cause respiratory tract infections that are typically mild, such as some cases of the common cold, though rarer forms can be lethal, such as severe acute respiratory syndrome (SARS) and Middle East respiratory syndrome (MERS).

There is currently an outbreak of respiratory disease caused by a novel coronavirus, named “severe acute respiratory syndrome coronavirus 2” (SARS-CoV-2) and the disease it causes has been named “Coronavirus Disease 2019” (COVID-19). The current pandemic caused by SARS-Cov-2 is a health emergency that requires the development of new vaccines and drugs to prevent or treat this disease. Notably, this pandemic has led to more than 800,000 deaths worldwide to 23 Aug. 2020, from over 23 million confirmed infections.

The severity of COVID-19 ranges from asymptomatic infection (estimated to be around 80% of infected individuals) to critical illness. The Chinese Center for Disease Control and Prevention reported that 14% of infected individuals developed severe disease (with dyspnea, hypoxia or greater than 50% lung involvement on imaging tests) and 5% developed critical disease (characterized by respiratory failure, systemic shock or multi-organ failure). It has been estimated that up to one third of hospitalized patients require mechanical ventilation in an intensive care unit, and mortality among those that are ventilated is approximately 80%.

One of the notable characteristics of COVID-19 is the complexity and heterogeneity of the disease, and the number of organs that it affects. SARS-CoV-2 typically enters the subject by the nose and/or throat, where it can attach to the cell surface receptor angiotensin-converting enzyme 2 (ACE2) and enter the cell. As the virus multiplies in the early phase, symptoms may be absent or may include fever, dry cough, loss of smell or taste, and sore throat, for example. If the infection progresses and the virus enters the lungs, the disease can worsen significantly, potentially resulting in sepsis, pneumonia, and acute respiratory distress syndrome (ARDS), and requiring oxygen support and/or ventilation. In biopsy or autopsy studies, diffuse alveolar damage with the formation of hyaline membranes, mononuclear cells, and macrophages infiltrating air spaces, and a diffuse thickening of the alveolar wall was observed (for review, see, Li et al., 2020. Lancet, 395:1517-1520).

However, it is not only the lung that is affected in COVID-19. The disease appears to affect the brain, with some patients presenting with encephalitis, seizures, loss of consciousness, or stroke; the heart and blood vessels, with some patients presenting with blood clots, cardiac inflammation and scarring, or cardiac arrest; the liver, with liver dysfunction evidenced by elevated levels of alanine aminotransferase and aspartate aminotransferase (AST); and the kidneys, with kidney damage evidenced by high levels of protein in the urine, requiring dialysis in some instances.

The scientific community, including critical industry and academic partnerships, have embarked on an unprecedented race against the COVID-19 pandemic to develop and produce effective vaccine(s) for global supply (Amanat, F. et al., 2020. Immunity 52:583-589). A number of recent reports validate the SARS-CoV-2 spike protein as a promising target for COVID-19 vaccine development (van Doremalen, N. et al., 2020. Nature, doi:10.1038/s41586-020-2608-y; Corbett, K. S. et al., 2020. N Engl J Med, doi:10.1056/NEJMoa2024671; Mercado, N. B. et al., 2020. Nature, doi:10.1038/541586-020-2607-z).

### SUMMARY

The present disclosure is based on the unexpected finding that replacement of an amino acid sequence corresponding to a furin-like cleavage site of the SARS-CoV-2 spike protein with a flexible linker leads to significant improvement in protein expression and stability of the modified spike protein. It has also been found that when the modified protein is fused to a structure-stabilizing domain that stabilizes the modified protein in a conformation that mimics a prefusion trimeric form of the SARS-CoV-2 spike protein, the resulting chimeric protein shows significant improvement in reactivity to conformational antibodies that bind specifically to the spike protein presented by SARS-CoV-2. These findings have been reduced to practice in polypeptides, compositions and methods for stimulating an immune response, including the development of a humoral and/or cellular immune response, to wild-type spike protein complexes of ACE2-interacting coronaviruses, including the native SARS-CoV-2 spike protein complex, as described hereafter.

Accordingly, disclosed herein, in one aspect, is a modified SARS-CoV-2 spike polypeptide that is distinguished from a wild-type SARS-CoV-2 spike protein by an absence of a furin cleavage site at a location corresponding to the furin cleavage site of the wild-type SARS-CoV-2 spike protein and a presence of a heterologous flexible linker at the location.

Suitably, the flexible linker connects first and second polypeptides, wherein the first polypeptide corresponds to an upstream portion of the wild-type SARS-CoV-2 spike protein and the second polypeptide corresponds to a downstream portion of the wild-type SARS-CoV-2 spike protein, wherein the carboxy-terminal residue of the upstream portion is immediately upstream of an amino acid corresponding to any one of Pro⁶⁸¹, Ser⁶⁸⁰, Asn⁶⁷⁹, Thr⁶⁷⁸, Gln⁶⁷⁷, and Thr⁶⁷⁶, and the amino-terminal residue of the downstream portion is immediately downstream of an amino acid corresponding to any one of Ser⁶⁸⁶, Val⁶⁸⁷, Ala⁶⁸⁸, Ser⁶⁸⁹, Gln⁶⁹⁰ and Ser⁶⁹¹ of the full-length wild-type SARS-CoV-2 spike protein amino acid sequence set forth in SEQ ID NO:1.

In some embodiments, the carboxy-terminal residue of the upstream portion is immediately downstream of an amino acid corresponding to any one of Gln⁶⁷⁵, Thr⁶⁷⁶, Gln⁶⁷⁷, Thr⁶⁷⁸, Asn⁶⁷⁹, and Ser⁶⁸⁰, and the amino-terminal residue of the downstream portion is immediately upstream of an amino acid corresponding to any one of Ser⁶⁹¹, Gln⁶⁹⁰, Ser⁶⁸⁹, Ala⁶⁸⁸, Val⁶⁸⁷, and Ser⁶⁸⁶ of the full-length wild-type SARS-CoV-2 spike protein amino acid sequence set forth in SEQ ID NO:1.

In some embodiments, the carboxy-terminal residue of the upstream portion correspond to an amino acid residue selected from Pro⁶⁸¹, Ser⁶⁸⁰, Asn⁶⁷⁹, Thr⁶⁷⁸, Gln⁶⁷⁷, Thr⁶⁷⁶ and Gln⁶⁷⁵, and the amino-terminal residue of the downstream portion corresponds to an amino acid residue selected from Ser⁶⁸⁶, Val⁶⁸⁷, Ala⁶⁸⁸, Ser⁶⁸⁹, Gln⁶⁹⁹ and Ser⁶⁹¹, wherein the amino acid numbering is relative to a full-length wild-type SARS-CoV-2 spike protein amino acid sequence set forth in SEQ ID NO:1.

In certain embodiments, the modified polypeptide lacks an amino acid residue corresponding to Pro⁶⁸¹ of the SARS-CoV-2 spike protein amino acid sequence set forth in SEQ ID NO:1.

In certain embodiments, the modified polypeptide lacks an amino acid residue corresponding to Ala⁶⁸⁴ of the SARS-CoV-2 spike protein amino acid sequence set forth in SEQ ID NO:1.

In preferred embodiments, the carboxyl-terminal residue of the upstream portion is an amino acid residue corresponding to Asn⁶⁷⁹, and the amino-terminal residue of the downstream portion is an amino acid residue corresponding to Ser⁶⁹¹ of the SARS-CoV-2 spike protein amino acid sequence set forth in SEQ ID NO:1.

In specific embodiments, the upstream and downstream portions of the wild-type SARS-CoV-2 spike protein comprise, consists or consist essentially of an amino acid sequence having at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98% or 99% sequence similarity or identity to the amino acid sequence set forth in SEQ ID NO:1.

In some of the same and other embodiments, the first and second polypeptides of the modified polypeptide have at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98% or 99% sequence similarity or identity to the amino acid sequence set forth in SEQ ID NO:1.

Suitably, the flexible linker consists or consists essentially of glycine and serine residues. In preferred embodiments, the flexible linker is selected from GSG, GGSG and GGSGG.

In certain embodiments, the flexible linker lacks one or both of a proline and an alanine.

The modified polypeptide may comprise, consist or consist essentially of a whole precursor of a SARS-CoV-2 spike protein or a portion thereof. In some embodiments the modified polypeptide lacks any one or more of an endogenous signal peptide, an endogenous head portion of the spike protein, an endogenous stem portion of the spike protein, an endogenous mucin-like domain, an endogenous membrane proximal external region, an endogenous fusion peptide, an endogenous transmembrane domain and an endogenous cytoplasmic tail corresponding to the SARS-CoV-2 spike protein. In preferred embodiments, the modified polypeptide corresponds to a SARS-CoV-2 spike protein ectodomain. The modified polypeptide suitably comprises at least one pre-fusion epitope that is not present in the post-fusion form of the SARS-CoV-2 spike protein.

The modified polypeptide may be operably connected downstream to a heterologous structure-stabilizing moiety to form a ‘chimeric polypeptide’. Suitably, the structure-stabilizing moiety stabilizes the modified polypeptide in a conformation that mimics the pre-fusion conformation of the wild-type SARS-CoV-2 spike protein. In representative embodiments of this type, the structure-stabilizing moiety inhibits the modified polypeptide from adopting a conformation that mimics the post-fusion conformation of the wild-type SARS-CoV-2 spike protein.

In a related aspect, the present disclosure provides a chimeric polypeptide comprising a modified polypeptide as broadly defined above and elsewhere herein, operably connected downstream to a heterologous structure-stabilizing moiety.

In some embodiments, the structure-stabilizing moiety comprises, consists or consists essentially of a trimerization domain, representative examples of which include, but are not limited to, catalytic subunit of Escherichia coli aspartate transcarbamoylase (ATCase), the ‘foldon’ trimerizing sequence from the bacteriophage T4 fibritin neck region peptide, human lung surfactant D protein, oligomerization coiled-coil adhesins and complementary heptad repeat regions of an enveloped virus class I fusion protein.

In preferred embodiments, the structure-stabilizing moiety comprises complementary first heptad repeat (HR1) and second heptad repeat (HR2) regions that associate with each other under conditions suitable for their association (e.g., in aqueous solution) to form an anti-parallel, two-helix bundle. The HR1 and HR2 regions suitably lack complementarity to the modified polypeptide, so that they preferentially form an anti-parallel, two-helix bundle with each other, rather than with structural elements of the modified polypeptide. In some embodiments, each of the HR1 and HR2 regions is independently characterized by a n-times repeated 7-residue pattern of amino acid types, represented as (a-b-c-d-e-f-g-)ₙ or (d-e-f-g-a-b-c-)ₙ, wherein the pattern elements ‘a’ to ‘g’ denote conventional heptad positions at which the amino acid types are located and n is a number equal to or greater than 2, and at least 50% (or at least 51% to at least 99% and all integer percentages in between) of the conventional heptad positions ‘a’ and ‘d’ are occupied by hydrophobic amino acid types and at least 50% (or at least 51% to at least 99% and all integer percentages in between) of the conventional heptad positions ‘b’, ‘c’, ‘e’, ‘f’ and ‘g’ are occupied by hydrophilic amino acid types, the resulting distribution between hydrophobic and hydrophilic amino acid types enabling the identification of the heptad repeat regions. In some embodiments, one or both of the HR1 and HR2 regions comprises, consists or consists essentially of an endogenous Class I enveloped virus fusion protein heptad repeat region amino acid sequence. In representative examples of this type, the HR1 and HR2 regions comprise, consist or consist essentially of complementary endogenous heptad repeat A (HRA) and heptad repeat B (HRB) regions, respectively, of one or more Class I enveloped virus fusion proteins. In some embodiments, the HRA region amino acid sequence and the HRB region amino acid sequence are derived from the same Class I enveloped virus fusion protein. In other embodiments, the HRA region amino acid sequence and the HRB region amino acid sequence are derived from the different Class I enveloped virus fusion proteins. In representative examples, the HR1 and HR2 regions are independently selected from HRA and HRB regions of fusion proteins expressed by orthomyxoviruses, paramyxoviruses, retroviruses, coronaviruses, filoviruses and arenaviruses. In exemplary embodiments, the HR1 and HR2 regions are derived from HIV GP160.

In specific embodiments, the structure-stabilizing moiety is operably connected directly or indirectly to the carboxy-terminal residue of an amino acid sequence corresponding to the SARS-CoV-2 ectodomain polypeptide. In some embodiments, the structure-stabilising moiety is operably connected indirectly to the carboxy-terminal residue via a flexible linker. Suitably, the carboxy-terminal residue corresponds to Gly¹²⁰⁴ of the SARS-CoV-2 spike protein.

In some embodiments, the HR1 and HR2 regions of the structure-stabilizing moiety are connected by a linker, which generally consists of about 1 to about 100 amino add residues (including all integer amino add residues therebetween). The linker may comprise at least one moiety selected from a purification moiety that facilitates purification of the chimeric polypeptide, an immune-modulating moiety that modulates an immune response to the chimeric polypeptide, a cell targeting moiety that directs the chimeric polypeptide to a specific cell subtype and a structural flexibility-conferring moiety.

In another aspect, the present disclosure provides a polynucleotide that comprises a coding sequence for a modified polypeptide or chimeric polypeptide, as broadly described above and elsewhere herein.

Yet another aspect of the present disclosure provides a nucleic acid construct that comprises a polynucleotide comprising a coding sequence for a modified polypeptide or chimeric polypeptide, as broadly described above and elsewhere herein, operably linked to a regulatory element that is operable in a host cell.

In a related aspect, the present disclosure provides a host cell that contains a nucleic acid construct, as broadly described above and elsewhere herein. The host cell may be a prokaryotic or eukaryotic host cell.

The modified polypeptides and chimeric polypeptides of the present disclosure can self-assemble under suitable conditions (e.g., in aqueous solution) to form a polypeptide complex. Accordingly, in another aspect, the present disclosure provides a method of producing a polypeptide complex, wherein the method comprises: combining modified polypeptides or chimeric polypeptides, as broadly defined above and elsewhere herein, under conditions (e.g., in aqueous solution) suitable for the formation of a polypeptide complex, whereby a polypeptide complex is produced that comprises three modified polypeptides or three chimeric polypeptides. In embodiments in which the modified polypeptide is operably connected to a structure-stabilizing moiety comprising complementary first heptad repeat (HR1) and second heptad repeat (HR2) regions that associate with each other under conditions suitable for their association (e.g., in aqueous solution) to form an anti-parallel, two-helix bundle, the polypeptide complex produced by the method is characterized by a six-helix bundle formed by oligomerization of the two-helix bundles of the respective structure-stabilizing moieties of the chimeric polypeptides.

In a related aspect, a polypeptide complex is disclosed herein that comprises a trimer of modified polypeptides or chimeric polypeptides, as broadly described above and elsewhere herein.

Disclosed herein in another aspect is a composition comprising a modified polypeptide, chimeric polypeptide, polypeptide complex, polynucleotide or nucleic acid construct, as broadly described above and elsewhere herein, and a pharmaceutically acceptable carrier, diluent or adjuvant. In specific embodiments, the composition is an immune-modulating composition.

The modified polypeptide and polypeptide complex of the present disclosure are useful for eliciting an immune response in subjects or production animals, to an ACE2-interacting coronavirus spike protein, including the spike protein of SARS-CoV-2, or complex thereof. Accordingly, another aspect of the present disclosure provides a method of eliciting an immune response to an ACE2-interacting coronavirus spike protein, or complex thereof, in a subject, wherein the method comprises administering to the subject an effective amount of a modified polypeptide, chimeric polypeptide, polypeptide complex, polynucleotide, nucleic acid construct or composition, as broadly described above and elsewhere herein.

In specific embodiments, the ACE2-interacting coronavirus is selected from SARS-CoV and SARS-CoV-2.

Disclosed herein in another aspect is a method of producing an antigen-binding molecule (e.g., an antibody such as a neutralizing antibody) that is immuno-interactive with a SARS-CoV-2 spike protein, or complex thereof, the method comprising: (1) screening a library of antigen-binding molecules with a modified polypeptide, chimeric polypeptide or polypeptide complex, as broadly described above and elsewhere herein; (2) detecting an antigen-binding molecule that binds specifically with the modified polypeptide or polypeptide complex; and (3) isolating the detected antigen-binding molecule. In some embodiments, the method further comprises (1) immunizing an animal with the modified polypeptide, polypeptide complex, or composition; (2) identifying and/or isolating a B cell from the animal, which is immuno-interactive with the fusion protein or complex thereof; and (3) producing the antigen-binding molecule expressed by that B cell.

The disclosure further provides an antigen-binding molecule produced by the above method, or a derivative antigen-binding molecule with the same epitope-binding specificity as the antigen-binding molecule. The derivative antigen-binding molecule may be selected from antibody fragments (such as Fab, Fab′, F(ab′)₂, Fv), single chain (scFv) and domain antibodies (including, for example, shark and camelid antibodies), and fusion proteins comprising an antibody, and any other modified configuration of the immunoglobulin molecule that comprises an antigen binding/recognition site.

A cell (e.g., a hybridoma or cell line) that produces the antigen-binding molecule, and an immune modulating composition comprising the antigen-binding molecule, as well as a pharmaceutically acceptable carrier, diluent or adjuvant are also provided.

The subject modified polypeptide, polypeptide complex, as well as the compositions and antigen-binding molecule, as broadly described above and elsewhere herein, are also useful for treating or preventing ACE2-interacting coronavirus infections, including SARS-CoV-2 infections. Accordingly, in yet another aspect, the present disclosure provides a method for treating, inhibiting the development of, or preventing an ACE2-interacting coronavirus infection, or at least one symptom, or viral shedding, associated therewith in a subject, wherein the method comprises administering to the subject an effective amount of a modified polypeptide, chimeric polypeptide, polypeptide complex, polynucleotide, nucleic acid construct, antigen-binding molecule or composition, as broadly described above and elsewhere herein.

In specific embodiments, the ACE2-interacting coronavirus is selected from SARS-CoV and SARS-CoV-2.

### BRIEF DESCRIPTION OF THE DRAWINGS

FIG. 1 is a graphical and diagrammatic representation showing antigen design and analysis. A, In vitro screening of S1/S2 linker modifications for yield and CR3022 affinity. B, linear representation of recombinant spike antigen, C, In vitro screening of signal sequence changes yield and CR3022 affinity D, In vitro screening of C-terminal length for yield and CR3022 affinity. E, Cryo-TEM reconstruction of antigen structure. F, Production yield from CHO cell culture transient expression, stable pools and clones in flasks or bioreactors. Red—antigen concentration in cell culture supernatant estimated by BIAcore. Black—Protein recovery following immunoaffinity purification measure by absorbance at 280 nm. G, Antigen stability assessed by CR3022 affinity following incubation at 4° C., 25° C., or 40° C. for up to 8 weeks. H, Antigen stability assessed by percentage trimer determined by SE-HPLC following incubation at 4° C., 25° C., or 40° C. for up to 8 weeks.

FIG. 2 is a diagrammatic representation of the sequence of the full-length SARS-CoV-2 spike and regions modified during targeted for production of the candidate vaccine panel.

FIG. 3 is a graphical representation showing the antibody response following SARS-COV-2 Sclamp vaccination in BALB/c mice. A, Prime/boost vaccination and bleed schedule for the study. B, SARS-CoV-2 Sclamp specific IgG EC₅₀ titer (reciprocal EC₅₀) in vaccinated mice 35- and 42-days following delivery of the first dose. C, MN titer against live SARS-CoV-2 (614D). D, MN titer against SARS-CoV-2 (614D) isolate and SARS-CoV-2 (614G) evaluated by PRNT₅₀ assay. E, MN titer against SARS-CoV-2 (614G) isolate evaluated by PRNT₅₀ assay. The p values were calculated using: 1) one-way ANOVA with Tukey's multiple comparison post-hoc test for normally distributed, homoscedastic data, 2) Welch's ANOVA with Games-Howell post-hoc analysis for all heteroscedastic data and 3) Kruskal-Wallis H test for non-normally distributed and homoscedastic data sets.

FIG. 4 is a graphical representation showing that Alhydrogel adjuvanted SARS-CoV-2 Sclamp vaccination elicits a superior Th cell, CTL and polyfunctional T cell response. A, Bar graphs show the mean (n=5)+SEM of the geometric mean fluorescent intensity (GMFI) of CD69 on gated B220+ cells in the FTA B, Bar graphs show the mean (n=5)+SEM percentage of peptides-pulsed targets that were killed in the FTA. C, Mean (n=5)+SEM of the number of the indicated cytokine producing S₁₋₁₂₂₆-specific CD4⁺ or CD8⁺ T cells in vaccinated or placebo control mice. D, Mean (n=5)+SEM of the number of the indicated cytokine producing S1-1226-specific CD8⁺ T cells in vaccinated or placebo control mice. For the ICS analysis, splenocytes recovered 24 days following the boost from mice prime/boost vaccinated at 2-weekly intervals were stimulated for 16 h with the Total pool as described in the Methods. Statistical significance P values for A and D are represented by * for p=0.05-0.01; ** for p=0.01-0.001, and *** for p=<0.001. For the paired data exhibiting a normal or non-normal distribution a paired t-test or Wilcoxon Signed-Rank test was used to calculate the p values respectively.

FIG. 5 is a graphical and photographic representation showing separation of SARS-Cov-2 Sclamp conformations by analytical SE-HPLC. A, Analytical SE-HPLC separation of low pH and high pH eluted SARS-CoV-2 Sclamp showing the presence of three peaks designate i, ii, and iii. B, Analytical SE-HPLC separation of SARS-CoV-2 Sclamp following 2-week incubation at 4° C. or 25° C. showing the presence of three peaks designated i, ii, and iii. C, Hypothesis describing the structure of SARS-CoV-2 Sclamp present at each peak present on the HPLC trace and how the antigen may transition between the two previously described Spike conformations, termed ‘open’ and ‘closed’, and a high molecular weight aggregated product.

FIG. 6 is a photographic representation showing cryo-EM single particle analysis of Sclamp. Purified SARS-CoV-2 Sclamp was plunge frozen on TEM grids and images by cryo-EM. Data was acquired on a CryoARM-300 equipped with a K3 camera. A, 2D class averages of the Sclamp particles with an imposed spherical mask of 250 Å were generated by RELION 3.1. B, Fourier shell correlation (FSC) analysis of single particle analysis 3D refinement with C3 symmetry, indicating a final resolution of 4.97 Å at a FSC cut-off of 0.143. C, Side-on and top down representations of the Sclamp cryo-EM map with the 3 S protein monomers colored individually for clarity. Sclamp cryo-EM map with the 3 S protein monomers colored individually for clarity.

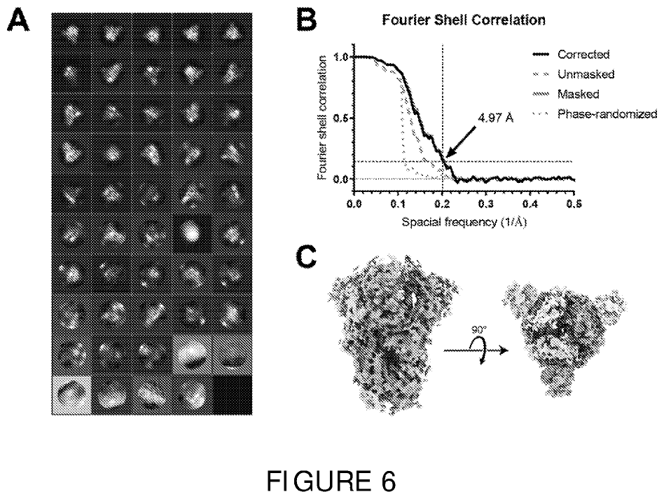

FIG. 7 is a graphical and photographic representation showing thermal stability and separation of SARS-Cov-2 Sclamp conformations by analytical SE-HPLC. Purified SARS-CoV-2 Sclamp was incubated for either 1 (A), 2 (B), 4 (C) or 8 (D) weeks at 4° C., 25° C. or before separation by SE-HPLC. (E) Negative stain images of SARS-CoV Sclamp stored for 4 weeks at 4° C., 25° C. or 40° C., samples are imaged using a Hitachi HT7700 microscope operated at 120 kV, at the magnification of 25,000× using high contrast mode.

FIG. 8 is a graphical representation showing virus neutralization in bronchoalveolar lavage (BAL). Day 42 BAL sample from SARS-CoV-2 Sclamp+Alhydrogel vaccinated BALB/c mice assessed by PRNT₅₀ assay against live SARS-CoV-2 (614D) isolate and SARS-CoV-2 (614G).

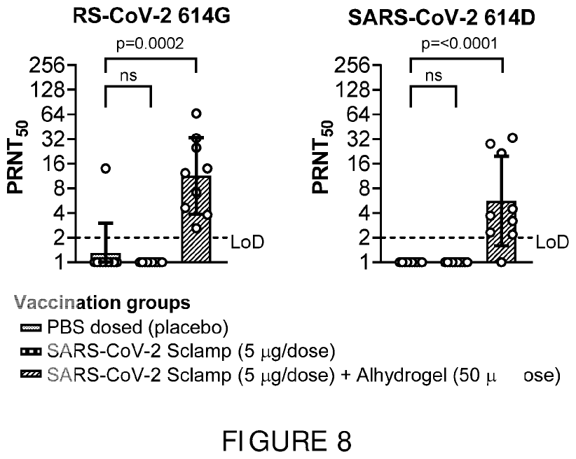

FIG. 9 is a graphical representation showing expression of IFN-γ, TNF-α, IL-2, IL-4 and/or IL-13 gated on CD3⁺CD4⁺ (top panel) or CD3⁺CD8⁺ (bottom panel) in placebo or SARS-CoV-2 Sclamp vaccinated mice.

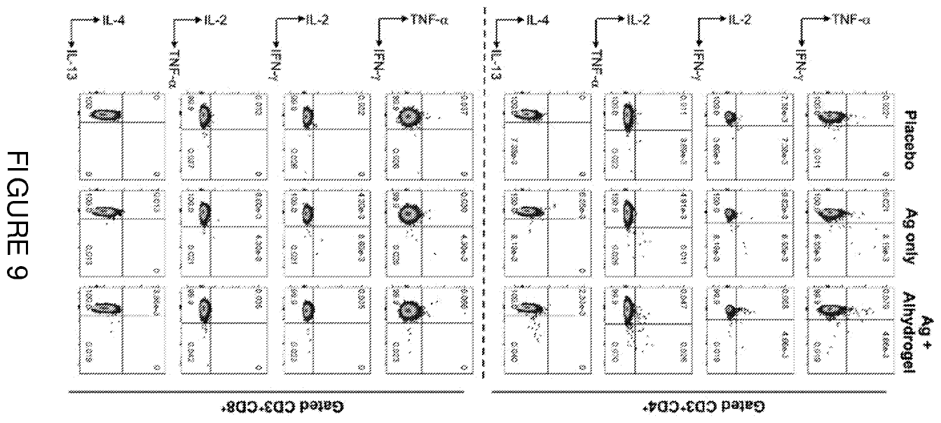

FIG. 10 is a graphical representation showing expression level SARS-CoV-2 S silenced clamp. Estimate based on BIAcore Standard curve using monoclonal antibody 2M10B11.

FIG. 11 is a graphical representation showing expression level SARS-CoV-2 S silenced clamp. Estimate based on BIAcore Standard curve using monoclonal antibody CR3022.

FIG. 12 is a graphical representation showing separation of SARS-Cov-2 Sclamp and S silenced clamp by SE-HPLC.

FIG. 13 is an SDS-PAGE analysis of purified SARS-Cov-2Sclamp and SARS-CoV-2-foldon. Molecular weight standards are shown in kDa on the left.

FIG. 14 is an ELISA assay showing reactivity of purified SARS-CoV-2 Sclamp (A) and SARS-CoV-2-foldon (B) to conformationally specific monoclonal antibodies including Spike RBD specific mAbs huCR3022 and DH1047, Spike NTD specific mAb 4A8, Spike S2 specific mAb 2.8. Only SARS-CoV-2 Sclamp reacts with anti-clamp mAb. No binding to control anti-influenza mAb C05 is observed for either SARS-CoV-2 Sclamp (A) or SARS-CoV-2-foldon (B).

### DETAILED DESCRIPTION OF THE DISCLOSURE

### 1. Definitions

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

As used herein, “and/or” refers to and encompasses any and all possible combinations of one or more of the associated listed items, as well as the lack of combinations when interpreted in the alternative (or).

Further, the terms “about” and “approximate”, as used herein when referring to a measurable value such as an amount, dose, time, temperature, activity, level, number, frequency, percentage, dimension, size, amount, weight, position, length and the like, is meant to encompass variations of ±15%, ±10%, ±5%, ±1%, ±0.5%, or even ±0.1% of the specified amount, dose, time, temperature, activity, level, number, frequency, percentage, dimension, size, amount, weight, position, length and the like. In instances in which the terms “about” and “approximate” are used in connection with the location or position of regions within a reference polypeptide, these terms encompass variations of ± up to 20 amino acid residues, ± up to 15 amino acid residues, ± up to 10 amino acid residues, ± up to 5 amino acid residues, ± up to 4 amino acid residues, ± up to 3 amino acid residues, ± up to 2 amino acid residues, or even ±1 amino acid residue.

The term “adjuvant” as used herein refers to a compound that, when used in combination with a specific immunogen (e.g., a modified polypeptide, chimeric polypeptide, polypeptide complex, polynucleotide and nucleic acid construct of the present disclosure) in a composition, will augment the resultant immune response, including intensification or broadening the specificity of either or both antibody and cellular immune responses. In the context of the present disclosure, an adjuvant will preferably enhance the specific immunogenic effect of the active agents of the present disclosure. The term “adjuvant” is typically understood not to comprise agents which confer immunity by themselves. An adjuvant assists the immune system unspecifically to enhance the antigen-specific immune response by e.g., promoting presentation of an antigen to the immune system or induction of an unspecific innate immune response. Furthermore, an adjuvant may preferably e.g., modulate the antigen-specific immune response by e.g., shifting the dominating Th2-based antigen specific response to a more Th1-based antigen specific response or vice versa. Accordingly, an adjuvant may favorably modulate cytokine expression/secretion, antigen presentation, type of immune response etc.

As used herein, the term “antigen” and its grammatically equivalent expressions (e.g., “antigenic”) refer to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity. Representative antigen-binding molecules that are useful in the practice of the present disclosure include polyclonal and monoclonal antibodies as well as their fragments (such as Fab, Fab′, F(ab′)₂, Fv), single chain (scFv) and domain antibodies (including, for example, shark and camelid antibodies), and fusion proteins comprising an antibody, and any other modified configuration of the immunoglobulin molecule that comprises an antigen binding/recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called α, δ, ε, γ, and μ, respectively. The subunit structures and three-dimensional configurations of different classes of immunoglobulins are well known. Antigen-binding molecules also encompass dimeric antibodies, as well as multivalent forms of antibodies. In some embodiments, the antigen-binding molecules are chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, for example, U.S. Pat. No. 4,816,567; and Morrison et al., 1984, Proc. Natl. Acad. Sci. USA 81:6851-6855). Also contemplated, are humanized antibodies, which are generally produced by transferring complementarity determining regions (CDRs) from heavy and light variable chains of a non-human (e.g., rodent, preferably mouse) immunoglobulin into a human variable domain. Typical residues of human antibodies are then substituted in the framework regions of the non-human counterparts. The use of antibody components derived from humanized antibodies obviates potential problems associated with the immunogenicity of non-human constant regions. General techniques for cloning non-human, particularly murine, immunoglobulin variable domains are described, for example, by Orlandi et al. (1989, Proc. Natl. Acad. Sci. USA 86: 3833). Techniques for producing humanized monoclonal antibodies are described, for example, by Jones et al. (1986, Nature 321:522), Carter et al. (1992, Proc. Natl. Acad. Sci. USA 89: 4285), Sandhu (1992, Crit. Rev. Biotech. 12: 437), Singer et al. (1993, J. Immun. 150: 2844), Sudhir (ed., Antibody Engineering Protocols, Humana Press, Inc. 1995), Kelley (“Engineering Therapeutic Antibodies,” in Protein Engineering: Principles and Practice Cleland et al. (eds.), pages 399-434 (John Wiley & Sons, Inc. 1996), and by Queen et al., U.S. Pat. No. 5,693,762 (1997). Humanized antibodies include “primatized” antibodies in which the antigen-binding region of the antibody is derived from an antibody produced by immunizing macaque monkeys with the antigen of interest. Also contemplated as antigen-binding molecules are humanized antibodies.

The term “anti-parallel”, as used herein, refers to a proteinaceous polymer in which regions or segments of the polymer are in a parallel orientation but have opposite polarities.

As used herein, the term “binds specifically” refers to a binding reaction which is determinative of the presence of a chimeric polypeptide or complex of the present disclosure in the presence of a heterogeneous population of molecules including macromolecules such as proteins and other biologics. In specific embodiments, the term “binds specifically” when referring to an antigen-binding molecule is used interchangeably with the term “specifically immuno-interactive” and the like to refer to a binding reaction which is determinative of the presence of a chimeric polypeptide or complex of the present disclosure in the presence of a heterogeneous population of proteins and other biologics. Under designated assay conditions, a molecule binds specifically to a chimeric polypeptide or complex of the disclosure and does not bind in a significant amount to other molecules (e.g., proteins or antigens) present in the sample. In antigen-binding molecule embodiments, a variety of immunoassay formats may be used to select antigen-binding molecules that are specifically immuno-interactive with a chimeric polypeptide or complex of the disclosure. For example, solid-phase ELISA immunoassays are routinely used to select monoclonal antibodies that are specifically immuno-interactive with a protein. See Harlow and Lane (1988) Antibodies, A Laboratory Manual, Cold Spring Harbor Publications, New York, for a description of immunoassay formats and conditions that can be used to determine specific immunoreactivity

The term “chimeric”, when used in reference to a molecule, means that the molecule contains portions that are derived from, obtained or isolated from, or based upon two or more different origins or sources. Thus, a polypeptide is chimeric when it comprises two or more amino acid sequences of different origin and includes (1) polypeptide sequences that are not found together in nature (i.e., at least one of the amino acid sequences is heterologous with respect to at least one of its other amino acid sequences), or (2) amino acid sequences that are not naturally adjoined.

By “coding sequence” is meant any nucleic acid sequence that contributes to the code for the polypeptide product of a gene or for the final mRNA product of a gene (e.g. the mRNA product of a gene following splicing). By contrast, the term “non-coding sequence” refers to any nucleic acid sequence that does not contribute to the code for the polypeptide product of a gene or for the final mRNA product of a gene.

The terms “coiled coil” or “coiled coil structure” are used interchangeably herein to refer to a structural motif in proteins, in which two or more α-helices (most often 2-7 α-helices) are coiled together like the strands of a rope (dimers and trimers are the most common types). Many coiled coil type proteins are involved in important biological functions such as the regulation of gene expression e.g., transcription factors. Coiled coils often, but not always, contain a repeated pattern, hpphppp or hppphpp, of hydrophobic (h) and polar (p) amino-acid residues, referred to as a heptad repeat (see herein below). Folding a sequence with this repeating pattern into an α-helical secondary structure causes the hydrophobic residues to be presented as a ‘stripe’ that coils gently around the helix in left-handed fashion, forming an amphipathic structure. The most favorable way for two such helices to arrange themselves in a water-filled environment of is to wrap the hydrophobic strands against each other sandwiched between the hydrophilic amino acids. It is thus the burial of hydrophobic surfaces, which provides the thermodynamic driving force for oligomerization of the α-helices. The packing in a coiled-coil interface is exceptionally tight. The α-helices may be parallel or anti-parallel, and usually adopt a left-handed super-coil. Although disfavored, a few right-handed coiled coils have also been observed in nature and in designed proteins. The terms “coiled coil” or “coiled coil structure” will be clear to the person skilled in the art based on the common general knowledge. Particular reference in this regard is made to review papers concerning coiled coil structures, such as for example, Cohen and Parry (1990. Proteins 7:1-15); Kohn and Hodges (1998. Trends Biotechnol 16:379-389); Schneider et al. (1998. Fold Des 3:R29-R40); Harbury et al. (1998. Science 282:1462-1467); Mason and Arndt (2004. Chem-BioChem 5:170-176); Lupas and Gruber (2005. Adv Protein Chem 70:37-78); Woolfson (2005. Adv Protein Chem 70:79-112); Parry et al. 2008. J Struct Biol 163:258-269); and Mcfarlane et al. (2009. Eur J Pharmacol 625:101-107).

As used herein the term “complementary” and grammatically equivalent expressions thereof refer to the characteristic of two or more structural elements (e.g., peptide, polypeptide, nucleic acid, small molecule, or portions thereof etc.) of being able to hybridize, oligomerize (e.g., dimerize), interact or otherwise form a complex with each other. For example, “complementary regions of a polypeptide” are capable of coming together to form a complex, which is characterized in specific embodiments by an anti-parallel, two-helix bundle.

As used herein, the term “complex” refers to an assemblage or aggregate of molecules (e.g., peptides, polypeptides, etc.) in direct and/or indirect contact with one another. In specific embodiments, “contact”, or more particularly, “direct contact” means two or more molecules are close enough so that attractive noncovalent interactions, such as Van der Waal forces, hydrogen bonding, ionic and hydrophobic interactions, and the like, dominate the interaction of the molecules. In such embodiments, a complex of molecules (e.g., a peptide and polypeptide) is formed under conditions such that the complex is thermodynamically favored (e.g., compared to a non-aggregated, or non-complexed, state of its component molecules). As used herein the term “complex”, unless described as otherwise, refers to the assemblage of two or more molecules (e.g., peptides, polypeptides or a combination thereof). In specific embodiments, the term “complex” refers to the assemblage of three polypeptides.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, the terms “conjugated”, “linked”, “fused” or “fusion” and their grammatical equivalents, in the context of joining together of two more elements or components or domains by whatever means including chemical conjugation or recombinant means (e.g., by genetic fusion) are used interchangeably. Methods of chemical conjugation (e.g., using heterobifunctional crosslinking agents) are known in the art. More specifically, as used herein, an “modified polypeptide”-“structure-stabilizing moiety” fusion or conjugate refers to the genetic or chemical conjugation of the modified polypeptide, which is suitably in a metastable, pre-fusion conformation, to a structure-stabilizing moiety. In specific embodiments, the structure-stabilizing moiety is fused indirectly to a modified polypeptide, via a linker, such as a glycine-serine (gly-ser) linker. In other embodiments, the structure-stabilizing moiety is fused directly to a modified polypeptide disclosed herein.

A “conservative amino acid substitution” is one in which the amino acid residue is replaced with an amino acid residue having a similar side chain. Families of amino acid residues having similar side chains have been defined in the art, which can be generally sub-classified as follows:

TABLE 1
AMINO ACID SUB-CLASSIFICATION

| SUB-CLASSES | AMINO ACIDS |
|---|---|
| Acidic | Aspartic acid, Glutamic acid |
| Basic | Noncyclic: Arginine, Lysine; Cyclic: Histidine |
| Charged | Aspartic acid, Glutamic acid, |
| | Arginine, Lysine, Histidine |
| Small | Glycine, Serine, Alanine, Threonine, Proline |
| Polar/neutral | Asparagine, Histidine, Glutamine, |
| | Cysteine, Serine, Threonine |
| Polar/large | Asparagine, Glutamine |
| Hydrophobic | Tyrosine, Valine, Isoleucine, Leucine, |
| | Methionine, Phenylalanine, Tryptophan |
| Aromatic | Tryptophan, Tyrosine, Phenylalanine |
| Residues that influence | Glycine and Proline |
| chain orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional polypeptide can readily be determined by assaying its activity. Conservative substitutions are shown in Table 2 under the heading of exemplary and preferred substitutions. Amino acid substitutions falling within the scope of the disclosure, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE 2
EXEMPLARY AND PREFERRED
AMINO ACID SUBSTITUTIONS

| ORIGINAL | EXEMPLARY | PREFERRED |
|---|---|---|
| RESIDUE | SUBSTITUTIONS | SUBSTITUTIONS |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

The term “construct” refers to a recombinant genetic molecule including one or more isolated nucleic acid sequences from different sources. Thus, constructs are chimeric molecules in which two or more nucleic acid sequences of different origin are assembled into a single nucleic acid molecule and include any construct that contains (1) nucleic acid sequences, including regulatory and coding sequences that are not found together in nature (i.e., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative constructs include any recombinant nucleic acid molecule such as a plasmid, cosmid, virus, autonomously replicating polynucleotide molecule, phage, or linear or circular single stranded or double stranded DNA or RNA nucleic acid molecule, derived from any source, capable of genomic integration or autonomous replication, comprising a nucleic acid molecule where one or more nucleic acid molecules have been operably linked. Constructs of the present disclosure will generally include the necessary elements to direct expression of a nucleic acid sequence of interest that is also contained in the construct, such as, for example, a target nucleic acid sequence or a modulator nucleic acid sequence. Such elements may include control elements such as a promoter that is operably linked to (so as to direct transcription of) the nucleic acid sequence of interest, and often includes a polyadenylation sequence as well. Within certain embodiments of the disclosure, the construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a host cell. Two or more constructs can be contained within a single nucleic acid molecule, such as a single vector, or can be containing within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in an organism or part thereof including a host cell. For the practice of the present disclosure, conventional compositions and methods for preparing and using constructs and host cells are well known to one skilled in the art, see for example, Molecular Cloning: A Laboratory Manual, 3^{rd} edition Volumes 1, 2, and 3. J. F. Sambrook, D. W. Russell, and N. Irwin, Cold Spring Harbor Laboratory Press, 2000.

By “corresponds to” or “corresponding to” is meant a nucleic acid sequence or an amino acid sequence that displays substantial sequence similarity or identity to a reference nucleic acid sequence or amino acid sequence, respectively. In general the nucleic acid sequence or amino acid sequence will display at least about 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to at least a portion of the reference nucleic acid sequence or amino acid sequence.

The term “domain”, as used herein, refers to a part of a molecule or structure that shares common physicochemical features, such as, but not limited to, hydrophobic, polar, globular and helical domains or properties such as ligand-binding, membrane fusion, signal transduction, cell penetration and the like. Often, a domain has a folded protein structure which has the ability to retain its tertiary structure independently of the rest of the protein. Generally, domains are responsible for discrete functional properties of proteins, and in many cases may be added, removed or transferred to other proteins without loss of function of the remainder of the protein and/or of the domain. Domains may be co-extensive with regions or portions thereof; domains may also include distinct, non-contiguous regions of a molecule. Examples of protein domains include, but are not limited to, a cellular or extracellular localization domain (e.g., signal peptide; SP), an immunoglobulin (Ig) domain, a membrane fusion (e.g., fusion peptide; FP) domain, an ectodomain, a membrane proximal external region (MPER) domain, a transmembrane (TM) domain, and a cytoplasmic (C) domain.

By “effective amount”, in the context of treating, inhibiting the development of, or preventing a condition is meant the administration of an amount of an agent or composition to an individual in need of such treatment, inhibition or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials. Non-limiting symptoms of coronavirus infections include acute febrile illness, malaise, fatigue, headache, flushing, diarrhea, nausea, vomiting, coughing including dry coughing, shortness of breath, difficulty in breathing, loss of smell, loss of taste, sore throat, runny nose, nasal congestion, and, in severe disease, pneumonia, acute respiratory distress syndrome, symptoms of systemic inflammatory response syndrome including production of pro-inflammatory mediators, vascular leakage and organ failure.

The term “endogenous” refers to a polypeptide or part thereof that is present and/or naturally expressed within a host organism or cell thereof. For example, an “endogenous” ectodomain polypeptide or part thereof refers to an ectodomain polypeptide of an enveloped fusion protein or a part of that ectodomain that is naturally expressed in enveloped virus.

As used herein, the term “endogenous HRA region” refers to an HRA region that is present in a Class I ectodomain polypeptide at substantially the same position as the HRA region in the amino acid sequence of the fusion protein precursor form of the naturally occurring fusion protein. The approximate amino acid positions of endogenous HRA regions of non-limiting examples of class I fusion proteins are listed in Table 3.

TABLE 3
APPROXIMATE POSITIONS OF HRA REGIONS
IN SELECTED CLASS I FUSION PROTEINS

HRA region

| Class I Fusion Protein | Start position | End position |
|---|---|---|
| Influenza A hemagglutinin (HA) | 356 | 390 |
| Influenza B HA | 383 | 416 |
| RSV Fusion protein (F) | 164 | 196 |
| hMPV F | 126 | 169 |
| PIV F | 136 | 168 |
| Measles F | 138 | 171 |
| Hendra | 136 | 169 |
| Nipah F | 136 | 169 |
| HIV glycoprotein (GP) 160 | 539 | 587 |
| Ebola GP | 557 | 593 |
| Marburg GP | 582 | 598 |
| SARS spike protein (S) | 892 | 1013 |
| MERS S | 984 | 1105 |

As used herein, the term “endogenous HRB region” refers to an HRB region that is present in a Class I ectodomain polypeptide at substantially the same position as the HRB region in the amino acid sequence of the fusion protein precursor form of the naturally occurring fusion protein. The approximate amino acid positions of endogenous HRB regions of non-limiting examples of class I fusion proteins are listed Table 4.

TABLE 4
APPROXIMATE POSITIONS OF HRB REGIONS
IN SELECTED CLASS I FUSION PROTEINS

HRB region

| Class I Fusion Protein | Start position | End position |
|---|---|---|
| Influenza A HA | 421 | 469 |
| Influenza B HA | 436 | 487 |
| RSV F | 488 | 524 |
| hMPV F | 456 | 490 |
| PIV F | 458 | 493 |
| Measles F | 454 | 493 |
| Hendra | 456 | 487 |
| Nipah F | 456 | 487 |
| HIV GP160 | 631 | 667 |
| Ebola GP | 600 | 635 |
| Marburg GP | 611 | 627 |
| SARS S | 1145 | 1187 |
| MERS S | 1248 | 1291 |

The term “endogenous production” refers to expression of a nucleic acid in an organism and the associated production and/or secretion of an expression product of the nucleic acid in the organism. In specific embodiments, the organism is multicellular (e.g., a vertebrate animal, preferably a mammal, more preferably a primate such as a human) and the nucleic acid is expressed within cells or tissues of the multicellular organism.

The terms “epitope” and “antigenic determinant” are used interchangeably herein to refer to an antigen, typically a protein determinant, that is capable of specific binding to an antibody (such epitopes are often referred to as “B cell epitopes”) or of being presented by a Major Histocompatibility Complex (MHC) protein (e.g., Class I or Class II) to a T-cell receptor (such epitopes are often referred to as “T cell epitopes”). Where a B cell epitope is a peptide or polypeptide, it typically comprises three or more amino acids, generally at least 5 and more usually at least 8 to 10 amino acids. The amino acids may be adjacent amino acid residues in the primary structure of the polypeptide (often referred to as contiguous peptide sequences), or may become spatially juxtaposed in the folded protein (often referred to as non-contiguous peptide sequences). T cell epitopes may bind to MHC Class I or MHC Class II molecules. Typically MHC Class I-binding T cell epitopes are 8 to 11 amino acids long. Class II molecules bind peptides that may be 10 to 30 residues long or longer, the optimal length being 12 to 16 residues. The ability of a putative T cell epitope to bind to an MHC molecule can be predicted and confirmed experimentally (Dimitrov et al., 2010. Bioinformatics 26(16):2066-8).

The term “flexible linker” as used herein refers to a proteinaceous molecule containing at least one amino acid residue, usually at least two amino acids residues joined by peptide bond(s), which molecule permits two polypeptides linked thereby to move more freely relative to one another, as compared to their movement without the flexible linker. In certain embodiments, the flexible linker provides increased rotational freedom for two polypeptides linked thereby than the two linked polypeptides would have in the absence of the flexible linker. Such freedom of relative movement or rotational freedom allows polypeptides joined by the flexible linker to perform their individual functions or elicit their activities with less structural hindrance. A flexible linker may be characterized by the absence of secondary structures such as helices or β-sheets or a maximal secondary structure content of 10%, 20% 30% or 40%. Non-limiting examples of flexible linkers include the amino acid sequences GS, GSG, GGSGG, GGSG, GSGS, AS, GGGS, G₄S, (G₄S)₂, (G₄S)₃, (G₄S)₄, G₄SG, GSGG and GSGGS. Additional flexible linker sequences are well known in the art. In various embodiments, the flexible linker contains or consists of at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 amino acid residues. In some of the same and other embodiments, the flexible linker contains or consists of up to 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 amino acid residues. In some of the same and other embodiments, the flexible linker contains or consists of between about 1 to about 30 amino acid residues, between about 1 to about 25 amino acid residues, between about 1 to about 20 amino acid residues, between about 1 to about 15 amino acid residues, between about 1 to about 12 amino acid residues, between about 1 to about 10 amino acid residues, between about 1 to about 8 amino acid residues, between about 1 to about 6 amino acid residues, between about 1 to about 5 amino acid residues, between about 1 to about 4 amino acid residues, or between about 1 to about 3 amino acid residues. In some of the same and other embodiments, the flexible linker contains or consists of between about 2 to about 30 amino acid residues, between about 2 to about 25 amino acid residues, between about 2 to about 20 amino acid residues, between about 2 to about 15 amino acid residues, between about 2 to about 12 amino acid residues, between about 2 to about 10 amino acid residues, between about 2 to about 8 amino acid residues, between about 2 to about 6 amino acid residues, between about 2 to about 5 amino acid residues, or between about 2 to about 4 amino acid residues. In some of the same and other embodiments, the flexible linker contains or consists of between about 3 to about amino acid residues, between about 3 to about 25 amino acid residues, between about 3 to about 20 amino acid residues, between about 3 to about 15 amino acid residues, between about 3 to about 12 amino acid residues, between about 3 to about 10 amino acid residues, between about 3 to about 8 amino acid residues, between about 3 to about 6 amino acid residues, or between about 3 to about 5 amino acid residues. In certain embodiments, the flexible linker contains or consists of 1, 2, 3, 4, 5, 6, 7, 8, 9 or 10 amino acid residues.

As used herein, the terms “furin cleavage site” and “furin-like cleavage site” are used interchangeably herein to refer to a scissile bond together with adjacent or non-adjacent recognition elements, or both, sufficient for detectable proteolysis at the scissile bond by furin under conditions suitable for furin protease activity. Furin cleavage sites are well known in the art or can be defined by routine methods. See, e.g., Basak, A. et al., 2001. Biochem. J. 353:537-545; Bader, O. et al., 2008. BMC Microbiol. 8:116; Schilling, O. et al., 2008. Nat. Biotechnol. 26:685-694; Rawlings, N. D. et al., 2008. Nucleic Acids Res. 36(Database issue): D320-D325; Rawlings, N. D. et al., 2010. Nucleic Acids Res. 38(Database issue): D227-D233 (2010); Seider, N. G. et al., 2012. Nat. Rev. Drug Discov. 10.1038/nrd3699; Braun, E. et al., 2019. Clin. Transl. Immunol., 8:e1073; Izaguirre. G., 2019. Viruses 11 (2019), 10.3390/v11090837. Reference is also made to Coutard, B. et al., 2020. Antiviral Res. 176:104742, who identified a furin-like cleavage site in SARS-CoV-2.

The term “helix bundle” refers to a plurality of peptide helices that fold such that the helices are substantially parallel or anti-parallel to one another. A two-helix bundle has two helices folded such that they are substantially parallel or anti-parallel to one another. Likewise, a six-helix bundle has six helices folded such that they are substantially parallel or anti-parallel to one another. By “substantially parallel or anti-parallel” is meant that the helices are folded such that the side chains of the helices are able to interact with one another. For example, the hydrophobic side chains of the helices are able to interact with one another to form a hydrophobic core.

The term “heterologous” when used with reference to portions of a nucleic acid or protein indicates that the nucleic acid or protein comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source, or coding regions from different sources. Similarly, a heterologous protein indicates that the protein comprises two or more subsequences that are not found in the same relationship to each other in nature (e.g., a fusion protein).

The term “host” refers to any organism, or cell thereof, whether eukaryotic or prokaryotic into which a construct of the disclosure can be introduced. In particular embodiments, the term “host” refers to eukaryotes, including unicellular eukaryotes such as yeast and fungi as well as multicellular eukaryotes such as animals non-limiting examples of which include invertebrate animals (e.g., insects, cnidarians, echinoderms, nematodes, etc.); eukaryotic parasites (e.g., malarial parasites, such as Plasmodium falciparum, helminths, etc.); vertebrate animals (e.g., fish, amphibian, reptile, bird, mammal); and mammals (e.g., rodents, primates such as humans and non-human primates). Thus, the term “host cell” suitably encompasses cells of such eukaryotes as well as cell lines derived from such eukaryotes.

Reference herein to “immuno-interactive” includes reference to any interaction, reaction, or other form of association between molecules and in particular where one of the molecules is, or mimics, a component of the immune system.

As use herein, the term “immunogenic composition” or “immunogenic formulation” refers to a preparation which, when administered to a vertebrate, especially an animal such as a mammal, will induce an immune response.

By “linker” is meant a molecule or group of molecules (such as a monomer or polymer) that connects two molecules and often serves to place the two molecules in a desirable configuration.

As used herein, the term “meta-stable”, as used in the context of a protein (e.g., an enveloped virus ectodomain polypeptide), refers to a labile conformational state that rapidly converts to a more stable conformational state upon a change in conditions. For example, an enveloped virus fusion protein in a pre-fusion form is in a labile, meta-stable conformation, and converts to the more stable post-fusion conformation upon, e.g., fusion to a host cell.

As used herein, the term “moiety” refers to a portion of a molecule, which may be a functional group, a set of functional groups, and/or a specific group of atoms within a molecule, that is responsible for a characteristic chemical, biological, and/or medicinal property of the molecule.

The term “neutralizing antigen-binding molecule” refers to an antigen-binding molecule that binds to or interacts with a target molecule or ligand and prevents binding or association of the target antigen to a binding partner such as a receptor or substrate, thereby interrupting the biological response that otherwise would result from the interaction of the molecules. In the case of the instant disclosure a neutralizing antigen-binding molecule suitably associates with a metastable or pre-fusion form of a SARS-CoV-2 spike protein and preferably interferes or reduces binding and/or fusion of the spike protein to a cell membrane.

The term “oligomer” refers to a molecule that consists of more than one but a limited number of monomer units in contrast to a polymer that, at least in principle, consists of an unlimited number of monomers. Oligomers include, but are not limited to, dimers, trimers, tetramers, pentamers, hexamers, heptamers, octamers, nonamers, decamers and the like. An oligomer can be a macromolecular complex formed by non-covalent bonding of macromolecules like proteins. In this sense, a homo-oligomer would be formed by identical molecules and by contrast, a hetero-oligomer would be made of at least two different molecules. In specific embodiments, an oligomer of the disclosure is a trimeric polypeptide complex consisting of three polypeptide subunits. In these embodiments, the trimeric polypeptide may be a “homotrimeric polypeptide complex” consisting of three identical polypeptide subunits, or a “heterotrimeric polypeptide complex” consisting of three polypeptide subunits in which at least one subunit polypeptide is non-identical. A “polypeptide subunit” is a single amino acid chain or monomer that in combination with two other polypeptide subunits forms a trimeric polypeptide complex.

The term “operably connected” or “operably linked” as used herein refers to a juxtaposition wherein the components so described are in a relationship permitting them to function in their intended manner. For example, a regulatory sequence (e.g., a promoter) “operably linked” to a nucleotide sequence of interest (e.g., a coding and/or non-coding sequence) refers to positioning and/or orientation of the control sequence relative to the nucleotide sequence of interest to permit expression of that sequence under conditions compatible with the control sequence. The control sequences need not be contiguous with the nucleotide sequence of interest, so long as they function to direct its expression. Thus, for example, intervening non-coding sequences (e.g., untranslated, yet transcribed, sequences) can be present between a promoter and a coding sequence, and the promoter sequence can still be considered “operably linked” to the coding sequence. Likewise, “operably connecting” a modified polypeptide as described herein to a heterologous, structure-stabilizing moiety encompasses positioning and/or orientation of the structure-stabilizing moiety such that the complementary HR1 and HR2 regions are permitted to associate with each other under conditions suitable for their association (e.g., in aqueous solution) to form an anti-parallel, two-helix bundle.

The terms “patient”, “subject”, “host” or “individual” used interchangeably herein, refer to any subject, particularly a vertebrate subject, and even more particularly a mammalian subject, for whom therapy or prophylaxis is desired. Suitable vertebrate animals that fall within the scope of the disclosure include, but are not restricted to, any member of the subphylum Chordata including primates (e.g., humans, monkeys and apes, and includes species of monkeys such from the genus Macaca (e.g., cynomolgus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. A preferred subject is a human, particularly a human in need of eliciting an immune response to a SARS-CoV-2 spike protein, or complex thereof. However, it will be understood that the aforementioned terms do not imply that symptoms are present.

By “pharmaceutically acceptable carrier” is meant a solid or liquid filler, diluent or encapsulating substance that can be safely used in topical or systemic administration to an animal, preferably a mammal, including humans. Representative pharmaceutically acceptable carriers include any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient(s), its use in the pharmaceutical compositions is contemplated.

The term “polynucleotide” or “nucleic acid” as used herein designates mRNA, RNA, cRNA, cDNA or DNA. The term typically refers to polymeric forms of nucleotides of at least 10 bases in length, either ribonucleotides or deoxynucleotides or a modified form of either type of nucleotide. The term includes single and double stranded forms of DNA.

“Polypeptide”, “peptide”, “protein” and “proteinaceous molecule” are used interchangeably herein to refer to molecules comprising or consisting of a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers.

As used herein, the term “post-fusion conformation” of a SARS-CoV-2 spike protein refers to the structure of the SARS-CoV-2 spike protein, which is in a terminal conformation (i.e., formed at the end of the fusion process with a host cell) and is the most energetically favorable state. In the post-fusion conformation, the fusion peptides or loops of the spike protein are brought into close proximity with the spike protein transmembrane domain. The post-fusion conformation of a SARS-CoV-2 spike protein is characterized by interaction between the endogenous HRA region and the endogenous HRB region of spike proteins to form a hairpin structure characterized by a six-helix bundle, comprising three endogenous HRB and three endogenous HRA regions. Post-fusion conformations of SARS-CoV-2 spike protein have been determined and are readily identifiable when viewed in negatively stained electron micrographs and/or by a lack of pre-fusion epitopes.

As used herein, the term “pre-fusion conformation” of a SARS-CoV-2 spike protein refers to the structure of a SARS-CoV-2 spike protein, which is in a meta-stable confirmation (i.e., in a semi-stable conformation that is not the most energetically favorable terminal conformation) and upon appropriate triggering is able to undergo conformational rearrangement to the terminal post-fusion conformation. Typically a pre-fusion conformations of SARS-CoV-2 spike protein contain an hydrophobic sequence, referred to as the fusion peptide or fusion loop, that is located internally within the pre-fusion conformation and cannot interact with either the viral or host cell membranes. Upon triggering, this hydrophobic sequence is inserted into the host cell membrane and the spike protein collapses into the post-fusion hairpin like conformation. The pre-fusion conformation of SARS-CoV-2 spike protein is characterized by non-interacting structural elements that subsequently associate in the energetically favorable post-fusion conformation. For example, the pre-fusion conformation of SARS-CoV-2 spike protein is dependent on the endogenous HRA region not interacting with the endogenous HRB region of individual fusion proteins of the trimer, thereby not permitting formation of a hairpin structure characterized by a six-helix bundle. Pre-fusion conformations of SARS-CoV-2 spike protein have been determined and are readily identifiable when viewed in negatively stained electron micrographs and/or by pre-fusion epitopes that are not present on post-fusion conformations.

“Regulatory elements”, “regulatory sequences”, control elements”, “control sequences” and the like are used interchangeably herein to refer to nucleotide sequences located upstream (5′ non-coding sequences), within, or downstream (3′ non-coding sequences) of a coding sequence, and which influence the transcription, RNA processing or stability, or translation of the associated coding sequence, either directly or indirectly. Regulatory elements include enhancers, promoters, translation leader sequences, introns, Rep recognition element, intergenic regions and polyadenylation signal sequences. They include natural and synthetic sequences as well as sequences which may be a combination of synthetic and natural sequences.

The term “replicon” refers to any genetic element, e.g., a plasmid, a chromosome, a virus, a cosmid, etc., that behaves as an autonomous unit of polynucleotide replication within a cell, i.e., capable of replication under its own control.

As used herein, “SARS-CoV-2 spike protein ectodomain polypeptide” refers to a polypeptide that contains a virion surface exposed portion of a mature SARS-CoV-2 spike protein, with or without the signal peptide but lacks the transmembrane domain and cytoplasmic tail of the naturally occurring or reference SARS-CoV-2 spike protein.

“Self-assembly” refers to a process of spontaneous assembly of a higher order structure that relies on the natural attraction of the components of the higher order structure (e.g., molecules) for each other. It typically occurs through random movements of the molecules and formation of bonds based on size, shape, composition, or chemical properties.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

“Similarity” refers to the percentage number of amino acids that are identical or constitute conservative substitutions as defined in Tables 1 and 2 supra. Similarity may be determined using sequence comparison programs such as GAP (Deveraux et al. 1984, Nucleic Acids Research 12: 387-395). In this way, sequences of a similar or substantially different length to those cited herein might be compared by insertion of gaps into the alignment, such gaps being determined, for example, by the comparison algorithm used by GAP.

Terms used to describe sequence relationships between two or more polynucleotides or polypeptides include “reference sequence,” “comparison window”, “sequence identity,” “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

As used herein, the term “single-chain” refers to a molecule comprising amino acid monomers linearly linked by peptide bonds.

A “trimerization domain”, as used herein, refers to a protein domain that preferentially interacts or associates with one or more other protein domains directly or via a bridging molecule, wherein the interaction of the other protein domains substantially contribute to or efficiently promote trimerization (i.e., the formation of a trimer, which may be a homotrimer or heterotrimer). Representative trimerization domains include the catalytic subunit of Escherichia coli aspartate transcarbamoylase (ATCase), the ‘foldon’ trimerizing sequence from the bacteriophage T4 fibritin neck region peptide, human lung surfactant D protein, oligomerization coiled-coil adhesins, complementary heptad repeat regions of an enveloped virus class I fusion protein, etc.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “wild-type”, “native” and “naturally occurring” are used interchangeably herein to refer to a gene or gene product that has the characteristics of that gene or gene product when isolated from a naturally occurring source. A wild type, native or naturally occurring gene or gene product (e.g., a polypeptide) is that which is most frequently observed in a population and is thus arbitrarily designed the “normal” or “wild-type” form of the gene or gene product.

Each embodiment described herein is to be applied mutatis mutandis to each and every embodiment unless specifically stated otherwise.

### 2. Modified Polypeptides

The present disclosure is predicated in part on the discovery that replacing the furin-like cleavage site of the SARS-CoV-2 spike protein, which is located at about aa680 to about aa685 of the full-length spike protein amino acid sequence, with a flexible linker leads to significant improvement in protein expression and stability of the modified spike protein. When the modified protein is fused to a structure-stabilizing domain that stabilizes the modified protein in a conformation that mimics a prefusion trimeric form of the wild-type spike protein, the resulting chimeric protein shows significant improvement in reactivity to conformational antibodies that bind specifically to the spike protein presented by native SARS-CoV-2. These improvements are considered beneficial for facilitating large-scale manufacture and storage of a vaccine against SARS-CoV-2 and for eliciting effective immunity to SARS-CoV-2 and for treating or inhibiting the development of symptoms associated with COVID-19.

Accordingly, the present disclosure provides a modified SARS-CoV-2 spike polypeptide that is distinguished from a wild-type SARS-CoV-2 spike protein by an absence of a furin cleavage site at a location corresponding to the furin cleavage site of the wild-type SARS-CoV-2 spike protein and a presence of a heterologous flexible linker at the location. The flexible linker is typically connected directly or indirectly to a first polypeptide and to a second polypeptide, wherein the first polypeptide corresponds to an upstream portion of the wild-type SARS-CoV-2 spike protein and the second polypeptide corresponds to a downstream portion of the wild-type SARS-CoV-2 spike protein.

The carboxy-terminal residue of the upstream portion may be immediately upstream of an amino acid corresponding to any one of Pro⁶⁸¹, Ser⁶⁸⁰, Asn⁶⁷⁹, Thr⁶⁷⁸, Gln⁶⁷⁷, and Thr⁶⁷⁶, and the amino-terminal residue of the downstream portion may be immediately downstream of an amino acid corresponding to any one of Ser⁶⁸⁶, Val⁶⁸⁷, Ala⁶⁸⁸, Ser⁶⁸⁹, Gln⁶⁹⁰ and Ser⁶⁹¹ of the full-length wild-type SARS-CoV-2 spike protein amino acid sequence set forth in SEQ ID NO:1. In some embodiments, the carboxy-terminal residue of the upstream portion is immediately downstream of an amino acid corresponding to any one of Gln⁶⁷⁵, Thr⁶⁷⁶, Gln⁶⁷⁷, Thr⁶⁷⁸, Asn⁶⁷⁹, and Ser⁶⁸⁰, and the amino-terminal residue of the downstream portion is immediately upstream of an amino acid corresponding to any one of Ser⁶⁹¹, Gln⁶⁹⁰, Ser⁶⁸⁹, Ala⁶⁸⁸, Val⁶⁸⁷, and Ser⁶⁸⁶ of the following full-length wild-type SARS-CoV-2 spike protein amino acid sequence:

[SEQ ID NO: 1]
MFVFLVLLPLVSSQCVNLTTRTQLPPAYTNSFTRGVYYPDKVFRSSVL
HSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVY
YHKNNKSWMESEFRVYSSANNCTFEYVSQPFLMDLEGKQGNFKNLREF
VFKNIDGYFKIYSKHTPINLVRDLPQGFSALEPLVDLPIGINITRFQT
LLALHRSYLTPGDSSSGWTAGAAAYYVGYLQPRTFLLKYNENGTITDA
VDCALDPLSETKCTLKSFTVEKGIYQTSNFRVQPTESIVRFPNITNLC
PFGEVFNATRFASVYAWNRKRISNCVADYSVLYNSASFSTFKCYGVSP
TKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPK
KSTNLVKNKCVNFNFNGLTGTGVLTESNKKFLPFQQFGRDIADTTDAV
RDPQTLEILDITPCSFGGVSVITPGTNTSNQVAVLYQDVNCTEVPVAI
HADQLTPTWRVYSTGSNVFQTRAGCLIGAEHVNNSYECDIPIGAGICA
SYQTQTNSPRRARSVASQSIIAYTMSLGAENSVAYSNNSIAIPTNFTI
SVTTEILPVSMTKTSVDCTMYICGDSTECSNLLLQYGSFCTQLNRALT
GIAVEQDKNTQEVFAQVKQIYKTPPIKDFGGFNFSQILPDPSKPSKRS
FIEDLLFNKVTLADAGFIKQYGDCLGDIAARDLICAQKFNGLTVLPPL
LTDEMIAQYTSALLAGTITSGWTFGAGAALQIPFAMQMAYRFNGIGVT
QNVLYENQKLIANQFNSAIGKIQDSLSSTASALGKLQDVVNQNAQALN
TLVKQLSSNFGAISSVLNDILSRLDKVEAEVQIDRLITGRLQSLQTYV
TQQLIRAAEIRASANLAATKMSECVLGQSKRVDFCGKGYHLMSFPQSA
PHGVVFLHVTYVPAQEKNFTTAPAICHDGKAHFPREGVFVSNGTHWFV
TQRNFYEPQIITTDNTFVSGNCDVVIGIVNNTVYDPLQPELDSFKEEL
DKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCC
SCGSCCKFDEDDSEPVLKGVKLHYT;

wherein:
   Italicized text corresponds to the signal peptide of SARS-CoV-2 spike protein;
   Bold text corresponds to a canonical furin-like cleavage site identified by Coutard, B. et al., 2020. supra; and
   Underlined text corresponds to transmembrane and cytoplasmic domains of SARS-CoV-2 spike protein.

In some embodiments, the carboxy-terminal residue of the upstream portion is selected from Pro⁶⁸¹, Ser⁶⁸⁰, Asn⁶⁷⁹, Thr⁶⁷⁸, Gln⁶⁷⁷, Thr⁶⁷⁶ and Gln⁶⁷⁵, and the amino-terminal residue of the downstream portion is selected from Ser⁶⁸⁶, Val⁶⁸⁷, Ala⁶⁸⁸, Ser⁶⁸⁹, Gln⁶⁹⁰ and Ser⁶⁹¹, wherein the amino acid numbering is relative to a full-length wild-type SARS-CoV-2 spike protein amino acid sequence set forth in SEQ ID NO:1.

In representative examples of this type, the carboxy-terminal residue of the upstream portion and the amino-terminal residue of the downstream portion are selected from the following:

| Carboxy-terminal residue of | Amino-terminal residue of the |
|---|---|
| upstream portion | downstream portion |
| Pro⁶⁸¹ | Ser⁶⁸⁶ |
| Pro⁶⁸¹ | Val⁶⁸⁷ |
| Pro⁶⁸¹ | Ala⁶⁸⁸ |
| Pro⁶⁸¹ | Ser⁶⁸⁹ |
| Pro⁶⁸¹ | Gln⁶⁹⁰ |
| Pro⁶⁸¹ | Ser⁶⁹¹ |
| Ser⁶⁸⁰ | Ser⁶⁸⁶ |
| Ser⁶⁸⁰ | Val⁶⁸⁷ |
| Ser⁶⁸⁰ | Ala⁶⁸⁸ |
| Ser⁶⁸⁰ | Ser⁶⁸⁹ |
| Ser⁶⁸⁰ | Gln⁶⁹⁰ |
| Ser⁶⁸⁰ | Ser⁶⁹¹ |
| Asn⁶⁷⁹ | Ser⁶⁸⁶ |
| Asn⁶⁷⁹ | Val⁶⁸⁷ |
| Asn⁶⁷⁹ | Ala⁶⁸⁸ |
| Asn⁶⁷⁹ | Ser⁶⁸⁹ |
| Asn⁶⁷⁹ | Gln⁶⁹⁰ |
| Asn⁶⁷⁹ | Ser⁶⁹¹ |
| Thr⁶⁷⁸ | Ser⁶⁸⁶ |
| Thr⁶⁷⁸ | Val⁶⁸⁷ |
| Thr⁶⁷⁸ | Ala⁶⁸⁸ |
| Thr⁶⁷⁸ | Ser⁶⁸⁹ |
| Thr⁶⁷⁸ | Gln⁶⁹⁰ |
| Thr⁶⁷⁸ | Ser⁶⁹¹ |
| Gln⁶⁷⁷ | Ser⁶⁸⁶ |
| Gln⁶⁷⁷ | Val⁶⁸⁷ |
| Gln⁶⁷⁷ | Ala⁶⁸⁸ |
| Gln⁶⁷⁷ | Ser⁶⁸⁹ |
| Gln⁶⁷⁷ | Gln⁶⁹⁰ |
| Gln⁶⁷⁷ | Ser⁶⁹¹ |
| Thr⁶⁷⁶ | Ser⁶⁸⁶ |
| Thr⁶⁷⁶ | Val⁶⁸⁷ |
| Thr⁶⁷⁶ | Ala⁶⁸⁸ |
| Thr⁶⁷⁶ | Ser⁶⁸⁹ |
| Thr⁶⁷⁶ | Gln⁶⁹⁰ |
| Thr⁶⁷⁶ | Ser⁶⁹¹ |
| Gln⁶⁷⁵ | Ser⁶⁸⁶ |
| Gln⁶⁷⁵ | Val⁶⁸⁷ |
| Gln⁶⁷⁵ | Ala⁶⁸⁸ |
| Gln⁶⁷⁵ | Ser⁶⁸⁹ |
| Gln⁶⁷⁵ | Gln⁶⁹⁰ |
| Gln⁶⁷⁵ | Ser⁶⁹¹ |

In certain embodiments, the modified polypeptide lacks an amino acid residue corresponding to Pro⁶⁸¹ of the SARS-CoV-2 spike protein amino acid sequence set forth in SEQ ID NO:1.

In some of the same and other embodiments, the modified polypeptide lacks an amino acid residue corresponding to Ala⁶⁸⁴ of the SARS-CoV-2 spike protein amino acid sequence set forth in SEQ ID NO:1.

In some of the same and other embodiments, an amino acid sequence corresponding to a wild-type SARS-CoV-2 spike protein amino acid sequence is not present in the modified polypeptide, wherein the amino acid sequence is selected from: 681-PRRAR-685 [SEQ ID NO:2], 681-PRRARS-686 [SEQ ID NO:3], 681-PRRARSV-687 [SEQ ID NO:4], 681-PRRARSVA-688 [SEQ ID NO:5], 681-PRRARSVAS-689 [SEQ ID NO:6], 681-PRRARSVASQ-690 [SEQ ID NO:7], 680-SPRRAR-685 [SEQ ID NO:8], 680-SPRRARS-686 [SEQ ID NO:9], 680-SPRRARSV-687 [SEQ ID NO:10], 680-SPRRARSVA-688 [SEQ ID NO:11], 680-SPRRARSVAS-689 [SEQ ID NO:12], 680-SPRRARSVASQ-690 [SEQ ID NO:13], 679-NSPRRAR-685 [SEQ ID NO:14], 680-NSPRRARS-686 [SEQ ID NO:15], 680-NSPRRARSV-687 [SEQ ID NO:16], 680-NSPRRARSVA-688 [SEQ ID NO:17], 680-NSPRRARSVAS-689 [SEQ ID NO:18], 680-NSPRRARSVASQ-690 [SEQ ID NO:19], 678-TNSPRRAR-685 [SEQ ID NO:20], 680-TNSPRRARS-686 [SEQ ID NO:21], 680-TNSPRRARSV-687 [SEQ ID NO:22], 680-TNSPRRARSVA-688 [SEQ ID NO:23], 680-TNSPRRARSVAS-689 [SEQ ID NO:24], 680-TNSPRRARSVASQ-690 [SEQ ID NO:25], 677-QTNSPRRAR-685 [SEQ ID NO:26], 680-QTNSPRRARS-686 [SEQ ID NO:27], 680-QTNSPRRARSV-687 [SEQ ID NO:28], 680-QTNSPRRARSVA-688 [SEQ ID NO:29], 680-QTNSPRRARSVAS-689 [SEQ ID NO:30], 680-QTNSPRRARSVASQ-690 [SEQ ID NO:31], 676-TQTNSPRRAR-685 [SEQ ID NO:32], 680-TQTNSPRRARS-686 [SEQ ID NO:33], 680-TQTNSPRRARSV-687 [SEQ ID NO:34], 680-TQTNSPRRARSVA-688 [SEQ ID NO:35], 680-TQTNSPRRARSVAS-689 [SEQ ID NO:36], and 680-TQTNSPRRARSVASQ-690 [SEQ ID NO:37], or an amino acid sequence corresponding (e.g., an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% sequence similarity or identity) to any of these sequences.

In preferred embodiments, the carboxyl-terminal residue of the upstream portion is an amino acid residue corresponding to Asn⁶⁷⁹ and the amino-terminal residue of the downstream portion is an amino acid residue corresponding to Ser⁶⁹¹ of the SARS-CoV-2 spike protein amino acid sequence set forth in SEQ ID NO:1, or an amino acid sequence corresponding thereto. In these embodiments, the sequence 680-SPRRARSVASQ-690 [SEQ ID NO:13] of the SARS-CoV-2 spike protein is not present in the modified polypeptide, or an amino acid sequence corresponding thereto (e.g., an amino acid sequence having at least 80%, 81%, 82%, 83%, 84%, 85%, 86%, 87%, 88%, 89%, 90%, 91%, 92%, 93%, 94%, 95%, 96%, 97%, 98% or 99% sequence similarity or identity).

In specific embodiments, the upstream and downstream portions of the wild-type SARS-CoV-2 spike protein comprise, consists or consist essentially of an amino acid sequence having at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98% or 99% sequence similarity or identity to the amino acid sequence set forth in SEQ ID NO:1.

In some of the same and other embodiments, the first and second polypeptides of the modified polypeptide have at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98% or 99% sequence similarity or identity to the amino acid sequence set forth in SEQ ID NO:1.

The flexible linker may comprise any suitable amino acid or amino acid sequence that spaces the upstream and downstream portions and permits the portions to move more freely relative to each other, as compared to their movement in the absence of the flexible linker. Flexible linkers are generally conformationally flexible in solution, and are suitably and predominantly composed of polar amino acid residue types. Typical (frequently used) amino acids in flexible linkers are serine and glycine. Less preferably, flexible linkers may also include alanine, threonine and proline. Thus, flexible linker is preferably flexible in conformation to ensure relaxed (unhindered) of the upstream and downstream portions of the modified polypeptide to adopt the same or similar structural conformation as the corresponding portions of the SARS-CoV-2 spike protein. Suitable linkers for use in the polypeptides envisaged herein will be clear to the skilled person, and may generally be any linker used in the art to link amino acid sequences, as long as the linkers are structurally flexible, in the sense that they do not affect a biological activity of the individual portions connected by the linker.

The skilled person will be able to determine the optimal linkers, optionally after performing a limited number of routine experiments. The flexible linker is suitably a proteinaceous molecule generally consisting of at least 1 amino acid residue and usually consisting of at least 2 amino acid residues, with a non-critical upper limit chosen for reasons of convenience being about 100 amino acid residues. In particular embodiments, the linker consists of about 1 to about 50 amino acid residues, or about 50 to about 100 amino acid residues, usually about 1 to about 40 amino acid residues, about 1 to about 30 amino acid residues, about 1 to about 20 amino acid residues, typically about 1 to about 10 amino acid residues, including all integers within these ranges. In specific embodiments, the flexible linker comprises 2, 3, 4, 5, 6, 7, 8, 9 or 10 amino acid residues. In particular, non-limiting embodiments, at least 50% of the amino acid residues of a linker sequence are selected from the group proline, glycine, and serine, more preferably glycine and serine. In further non-limiting embodiments, at least 60%, such as at least 70%, such as for example 80% and more particularly 90% of the amino acid residues of a linker sequence are selected from the group proline, glycine, and serine, more preferably glycine and serine. In other particular embodiments, the linker sequences essentially consist of polar amino acid residues; in such particular embodiments, at least 50%, such as at least 60%, such as for example 70% or 80% and more particularly 90% or up to 100% of the amino acid residues of a linker sequence are selected from the group consisting of glycine, serine, threonine, alanine, proline, histidine, asparagine, aspartic acid, glutamine, glutamic acid, lysine and arginine, more preferably glycine and serine. In some embodiments, linker sequences may include GG, GS, GSG, GGSG [SEQ ID NO: 76], GGSGG [SEQ ID NO: 77], [GGSG]ₙGG [SEQ ID NO: 78], [GGGGS]ₙ [SEQ ID NO: 79], [SSSG]ₙ [SEQ ID NO: 80], [SSSSG]ₙ [SEQ ID NO: 81], [AAPA]ₙ [SEQ ID NO: 82], [GGGKGGGG]ₙ [SEQ ID NO: 83], [GGGNGGGG]ₙ [SEQ ID NO: 84], [GGGCGGGG]ₙ [SEQ ID NO: 85], wherein n is an integer from 1 to 10, suitably 1 to 5, more suitably 1 to 3.

In specific embodiments, the flexible linker is selected from GSG, GGSG and GGSGG.

In certain embodiments, the flexible linker lacks one or both of a proline and an alanine.

The modified polypeptide may comprise, consist or consist essentially of a whole precursor of a SARS-CoV-2 spike protein or a portion thereof. In some embodiments the modified polypeptide lacks any one or more of an endogenous signal peptide, an endogenous head portion of the spike protein, an endogenous stem portion of the spike protein, an endogenous mucin-like domain, an endogenous membrane proximal external region, an endogenous fusion peptide, an endogenous transmembrane domain and an endogenous cytoplasmic tail, corresponding to the SARS-CoV-2 spike protein. In preferred embodiments, the modified polypeptide comprises, consists or consists essentially of an amino acid sequence corresponding to the a SARS-CoV-2 spike protein ectodomain, suitably lacking one or both of the endogenous transmembrane domain and endogenous cytoplasmic tail of the SARS-CoV-2 spike.

The modified polypeptide suitably comprises at least one pre-fusion epitope that is not present in the post-fusion form of the SARS-CoV-2 spike protein.

2.1 Chimeric Polypeptides

The present disclosure also contemplates a chimeric polypeptide comprising the modified polypeptide operably connected downstream to a heterologous structure-stabilizing moiety. The structure-stabilizing moiety is used to stabilize the modified polypeptide in a conformation that mimics the pre-fusion conformation of the wild-type SARS-CoV-2 spike protein, and generally inhibits the modified polypeptide from adopting a conformation that mimics the post-fusion conformation of the wild-type SARS-CoV-2 spike protein.

2.1.1 Structure-Stabilizing Moieties

Stabilization of the modified polypeptide typically requires a trimerization domain that is able to self-assemble to form a stable trimeric structure. Numerous trimerization domains are known in the art, including for example the catalytic subunit of Escherichia coli aspartate transcarbamoylase (ATCase), the ‘foldon’ trimerizing sequence from the bacteriophage T4 fibritin neck region peptide, human lung surfactant D protein, oligomerization coiled-coil adhesins and complementary heptad repeat regions of an enveloped virus class I fusion protein

Alternatively, a class of trimerization domains that can be used in the context of the present disclosure is found in the left-handed triple helix known as the collagen helix (Section 5.5.3 of Proteins by Creighton (ISBN 0-7167-2317-4). These triple helix-forming sequences involve a basic tripeptide repeat sequence of ¹Gly²Xaa³Xaa, where ²Xaa is often Pro, and ³Xaa is often 4-hydroxyproline. Although this motif is known as the “collagen” helix, it is found in many proteins beyond just collagen. The trimerization domain may thus be a sequence comprising multiple repeats of the sequence motif ¹Gly²Xaa³Xaa, which motif folds to form a helical structure that can trimerize with corresponding helical structures in other polypeptide chains.

Collagen also provides another class of trimerization domain. Zhang & Chen (J Biol Chem 274:22409-22413, 1999) describe a motif found in the non-collagenous domain 1 (NC1) of type X collagen, and this motif can be used for trimer and higher order oligomer formation without a triple helix. This trimeric association is highly thermostable without intermolecular disulfide bonds. The trimerization domain may thus comprise an NC1 sequence.

The trimerization domain (foldon) of the bacteriophage T4 protein fibritin (Tao et al., 1997. Structure 5:789-798; Gu the et al., 2004. J. Mol. Biol. 337, 905-915), in particular the C-terminal 27 to 30 residues of foldon, or a derivative thereof, may also be used to oligomerize a PD-L2 polypeptide. This trimerization domain may have the sequence GYIPEAPRDGQAYVRKDGEWVLLSTFL [SEQ ID NO: 38] or GSGYIPEAPRDGQAYVRKDGEWVLLSTFL [SEQ ID NO: 39]. Small modifications of this domain are also envisaged. Such modifications may be the substitution of Asp 9 by Cys for the purpose of the formation of a disulfide bridge between adjacent domains. Other modifications of surface amino acids of this domain may include substitutions of residues for optimizing the interactions at the interface between adjacent oligomerization domains such as hydrophobic, hydrophilic or ionic interactions or covalent bonds like disulfide bridges. Yet other modifications of surface amino acids of this domain may include substitutions of amino acids (e.g. by cysteine or lysine) for the generation of attachment sites for functional groups. A primary sequence of the SARS-CoV-2-foldon is provided in Example 6 and shown in SEQ ID NO: 99.

A common structural motif involved in protein trimerization is the coiled-coil domain, which occurs in a wide variety of proteins including motor proteins, DNA-binding proteins, extracellular proteins and viral fusion proteins (see, e.g., Burkhard et al., 2001. Trends Cell Biol 11:82-88). Coiled coils have been functionally characterized as folding (assembly, oligomerization) motifs, i.e., formation of a coiled coil structure drives in many instances the non-covalent association of different protein chains. Coiled coils have been structurally characterized as 2-, 3-, 4- or 5-stranded assemblies of α-helices arranged in parallel, anti-parallel or mixed topologies (see, e.g., Lupas, 1996. Trends Biochem Sci 21:375-382). Usually, the helices are slightly wrapped (coiled, wound) around each other in a left- or right-handed manner, termed supercoiling. It will be understood that the two-helix bundles of the present disclosure generally form coiled coil structures with a strong propensity to trimerize in order to form a highly stable six-helical coiled coil bundle.

In specific embodiments, the coiled coil domain is a leucine zipper, an illustrative example of which is derived from a nuclear protein that functions as a transcriptional activator of a family of genes involved in the General Control of Nitrogen (GCN4) metabolism in Saccharomyces cerevisiae. An exemplary sequence of such a GCN4 leucine zipper domain capable of forming a trimer is selected from:

[SEQ ID NO: 40]
RMKQIEDKIEEILSKIYHIENEIARIKKLIGE;
and
[SEQ ID NO: 41]
MKQIEDKIEEIESKQKKIENEIARIKK.

Alternative coiled-coil domains are those taken from bacterial transmembrane proteins, which form trimers. A suitable subset of transmembrane proteins is the adhesins (i.e., cell-surface proteins that mediate adhesion to other cells or to surfaces), and particularly non-fimbrial adhesins (e.g., in the oligomerization coiled-coil adhesins, or ‘Oca’, family). Specific sequences for use in the context of the present disclosure include those from Yersinia enterocolitica adhesin YadA, Neisseria meningitidis adhesin NadA, Moraxella catarrhalis surface protein UspA2, and other adhesins, such as the HadA adhesin from Hemophilus influenzae biogroup aegyptius etc. (see, SEQ ID NOs 28-31 and 42-58 of WO2006/011060). In addition, the eukaryotic heat-shock transcription factor has a coiled-coil trimerization domain that can be separately expressed and therefore used in the context of the present disclosure.

In specific embodiments, the coiled coil domain that forms the structure-stabilizing moiety, is in the form of a single-chain polypeptide comprising complementary first heptad repeat (HR1) and second heptad repeat (HR2) regions, as disclosed in International Application Publication No. WO 2018/176103. These complementary heptad repeats fold into an anti-parallel configuration, forming an anti-parallel, two-helix bundle that stabilizes an operably connected enveloped virus fusion polypeptide in a pre-fusion conformation. In preferred embodiments, the heptad repeats lack complementarity to the modified polypeptide and therefore preferentially associate with each other rather than with structural elements of the modified polypeptide, particularly structural elements corresponding to the endogenous heptad repeat regions of the SARS-CoV-2 spike protein. Association of the complementary heptad repeats of the structure-stabilizing moiety to one another under conditions suitable for their association (e.g., in aqueous solution) results in formation of an anti-parallel, two-helix bundle that inhibits rearrangement of the modified polypeptide to a post-fusion conformation. This two-helix bundle of the structure-stabilizing moiety can trimerize to form a highly stable six-helix bundle, thus permitting self-assembly of the chimeric polypeptide to form a modified polypeptide complex. The complex so assembled can mimic the pre-fusion conformation of a wild-type SARS-CoV-2 spike protein complex and comprises three chimeric polypeptides, characterized by a six-helix bundle formed by the coiled coil structures of the respective structure-stabilizing moieties of the chimeric polypeptides.

Heptad Repeats

Alpha-helical coiled coils have been characterized at the level of their amino acid sequences, in that, each helix is constituted of a series of heptad repeats. A heptad repeat (heptad unit, heptad) is a 7-residue sequence motif which can be encoded as hpphppp, and wherein each ‘h’ represents a hydrophobic residue and each ‘p’ is a polar residue. Occasionally, p-residues are observed at h-positions, and vice versa. A heptad repeat is also often encoded by the patterns a-b-c-d-e-f-g (abcdefg) or d-e-f-g-a-b-c (defgabc), in which case the indices ‘a’ to ‘g’ refer to the conventional heptad positions at which typical amino acid types are observed. By convention, indices ‘a’ and ‘d’ denote the positions of the core residues (central, buried residues) in a coiled coil. The typical amino acid types that are observed at core a- and d-positions are hydrophobic amino acid residue types; at all other positions (non-core positions), predominantly polar (hydrophilic) residue types are observed. Thus, conventional heptad patterns ‘hpphppp’ match with the pattern notation ‘abcdefg’ (‘hppphpp’ patterns match with the pattern notation ‘defgabc’, this notation being used for coiled coils starting with a hydrophobic residue at a d-position). The heptad repeat regions of the present disclosure include at least 2, and suitably 3 or more consecutive (uninterrupted) heptad repeats in individual α-helices of the coiled coil structure. Each series of consecutive heptad repeats in a helix is denoted a ‘heptad repeat sequence’ (HRS). The start and end of a heptad repeat sequence is preferably determined on the basis of the experimentally determined 3-dimensional (3-D) structure, if available. If a 3-D structure is not available, the start and end of a heptad repeat sequence is preferably determined on the basis of an optimal overlay of a (hpphppp)ₙ or (hppphpp)ₙ pattern with the actual amino acid sequence, where ‘h’ and ‘p’ denote hydrophobic and polar residues, respectively, and where ‘n’ is a number equal to or greater than 2. The start and end of each heptad repeat sequence is taken to be the first and last hydrophobic residue at an a- or d-position, respectively. Conventional H-residues are preferably selected from the group consisting of valine, isoleucine, leucine, methionine, phenylalanine, tyrosine, tryptophan, histidine, glutamine, threonine, serine and alanine, more preferably from the group consisting of valine, isoleucine, leucine and methionine, and most preferably isoleucine. Conventional p-residues are preferably selected from the group consisting of glycine, alanine, cysteine, serine, threonine, histidine, asparagine, aspartic acid, glutamine, glutamic acid, lysine and arginine. In case this method does not permit unambiguous assignment of amino acid residues to a heptad repeat sequence, a more specialized analysis method can be applied, such as the COILS method of Lupas et al. (1991. Science 252:1162-1164; http://www.russell.embl-heidelberg.de/cgi-bin/coils-svr.pl).

In particular embodiments, each heptad repeat region (HR1, HR2) is independently characterized by a n-times repeated 7-residue pattern of amino acid types, represented as (a-b-c-d-e-f-g-)ₙ or (d-e-f-g-a-b-c-)ₙ, as described for example in WO 2010/066740, the content of which is incorporated by reference herein in its entirety, wherein the pattern elements ‘a’ to ‘g’ denote conventional heptad positions at which the amino acid types are located and n is a number equal to or greater than 2, and at least 50% (or at least 51% to at least 99% and all integer percentages in between) of the conventional heptad positions ‘a’ and ‘d are occupied by hydrophobic amino acid types and at least 50% (or at least 51% to at least 99% and all integer percentages in between) of the conventional heptad positions ‘b’, ‘c’, ‘e’, ‘f’ and ‘g’ are occupied by hydrophilic amino acid types, the resulting distribution between hydrophobic and hydrophilic amino acid types enabling the identification of the heptad repeat regions. In specific embodiments, at least 50%, 70%, 90%, or 100% of the conventional heptad positions ‘a’ and ‘d’ are occupied by amino acids selected from the group consisting of valine, isoleucine, leucine, methionine or non-natural derivatives thereof. Since the latter amino acids correspond to more standard (more frequently observed) coiled coil core residues. In other embodiments, at least 50%, 70%, 90%, or 100% of the conventional heptad positions ‘a’ and ‘d’ are occupied by isoleucines. In some embodiments, at least 50%, 70%, 90%, or 100% of the conventional heptad positions ‘b’, ‘c’, ‘e’, ‘f’ and ‘g’ are occupied by amino acids selected from the group consisting of glycine, alanine, cysteine, serine, threonine, histidine, asparagine, aspartic acid, glutamine, glutamic acid, lysine, arginine or non-natural derivatives thereof. In illustrative examples of this type, the HR1 and HR2 regions comprise, consist or consist essentially of the sequence:

[SEQ ID NO: 42]
IEEIQKQIAAIQKQIAAIQKQIYRM

In particular embodiments, the HR1 and HR2 regions of the structure-stabilizing moiety (also referred to herein as “SSM”) comprise at least one endogenous heptad repeat of a Class I enveloped virus fusion protein. Suitably, the HR1 and HR2 regions are formed largely by complementary HRA and HRB regions, respectively, of one or more Class I enveloped virus fusion proteins. The HRA region amino acid sequence and the HRB region amino acid sequence may be derived from the same Class I enveloped virus fusion protein. Alternatively, they may be derived from the different Class I enveloped virus fusion proteins. In representative examples, the HR1 and HR2 regions are independently selected from HRA and HRB regions of orthomyxoviruses (e.g., Influenza A (Inf A), Influenza B (Inf B), Influenza C (Inf C)), paramyxoviruses (e.g., Measles (MeV), Rinderpest virus (RPV), Canine distemper virus (CDV), RSV, Human Metapneumovirus (HMPV), Parainfluenza virus (PIV), Mumps virus (MuV), Hendra virus (HeV), Nipah virus (NiV), Newcastle disease virus (NDV)), retroviruses (e.g., Human T cell leukemia virus type 1 (HTLV-1), HTLV-2, HTLV-3, HIV-1, HIV-2), filoviruses (e.g., Ebola virus (EBOV) including Zaire (ZEBOV), Reston (REBOV) and Sudan (SEBOV) strains, Marburg virus (MARV)), arenaviruses (e.g., Lassa virus (LASV), Lymphocytic choriomeningitis virus (LCMV), Junin virus (JUNV)), and coronaviruses (e.g., Human Coronavirus (HCoV), including HCoV 229E, HCoV OC43, HCoV HKU1, HCoV EMC, Human Torovirus (HToV), Middle East Respiratory Syndrome virus (MERS-CoV), Severe Acute Respiratory Syndrome virus (SARS-CoV)).

Exemplary HRA region amino acid sequences include, but are not limited to, those in Table 5:

TABLE 5
HRA REGION SEQUENCES OF
SELECTED CLASS I FUSION PROTEINS

| VIRUS | HRA REGION SEQUENCE |
|---|---|
| HIV | SGIVQQQNNLLRAIEAQQHLLQLTVWGIKQLQARILA |
| GP160 | [SEQ ID NO: 43] |
| | (GenPept dbjBAF31430.1) |
| RSV F | LHLEGEVNKIKSALLSTNKAVVSLGNGVSVLTSKVLD |
| | LK [SEQ ID NO: 44] |
| | (GenPept gbAHL84194.1) |
| HMPV F | IRLESEVTAIKNALKKTNEAVSTLGNGVRVLATAVRE |
| | LK [SEQ ID NO: 45] |
| | (GenPept gbAAN52913.1) |
| PIV F | KQARSDIEKLKEAIRDTNKAVQSVQSSIGNLIVAIKS |
| | VQ [SEQ ID NO: 46] |
| | (GenPept gbAAB21447.1) |
| MeV F | MLNSQAIDNLRASLETTNQAIEAIRQAGQEMILAVQG |
| | VQ [SEQ ID NO: 47] |
| | (GenPept dbjBAB60865.1) |
| HeV F | MKNADNINKLKSSIESTNEAVVKLQETAEKTVYVLTA |
| | LQ [SEQ ID NO: 48] |
| | (GenPept NP_047111.2) |
| Inf A | ENGWEGMVDGWYGFRHQNSEGTGQAADLKSTQAAI |
| HA | [SEQ ID NO: 49] |
| | (GenPept gbAEC23340.1) |
| Inf B | HGYTSHGAHGVAVAADLKSTQEAINKITKNLNYL |
| HA | [SEQ ID NO: 50] |
| | (GenPept gbAFH57854.1) |
| EBOV | GLRQLANETTQALQLFLRATTELRTFSILNRKAIDFL |
| GP | [SEQ ID NO: 51] |
| | (GenPept NP_066246.1) |
| MARV | LANQTAKSLELLLRVTTEERTFSLINRHAIDFLLTRW |
| GP | G [SEQ ID NO: 52] |
| | (GenPept YP_001531156.1) |
| MERS S | ISASIGDIIQRLDVLEQDAQIDRLINGRLTTLNAFVA |
| | QQLVRSESAALSAQLAKDKVNE [SEQ ID NO: 53] |
| | (GenPept gbAHX00711.1) |
| SARS S | ISSVLNDILSRLDKVEAEVQIDRLITGRLQSLQTYVT |
| | QQLIRAAEIRASANLAATKMSE [SEQ ID NO: 54] |
| | (GenPept gbAAR86788.1] |

Exemplary HRB region amino acid sequences include, but are not limited to, those in Table 6:

TABLE 6
HRB REGION SEQUENCES OF
SELECTED CLASS I FUSION PROTEINS

| VIRUS | HRB REGION SEQUENCE |
|---|---|
| HIV | HTTWMEWDREINNYTSLIHSLIEESQNQQEKNEQ |
| GP160 | ELLE [SEQ ID NO: 55] |
| | (GenPept dbjBAF31430.1) |
| RSV F | FDASISQVNEKINQSLAFIRKSDELLHNVNAGKS |
| | TTN [SEQ ID NO: 56] |
| | (GenPept gbAHL84194.1) |
| hMPV F | FNVALDQVFESIENSQALVDQSNRILSSAEKGNT |
| | G [SEQ ID NO: 57] |
| | (GenPept gbAAN52913.1] |
| PIV F | IELNKAKSDLEESKEWIRRSNQKLDSIGNWHQSS |
| | TT [SEQ ID NO: 58] |
| | (GenPept gbAAB21447.1) |
| MeV F | LERLDVGTNLGNAIAKLEDAKELLESSDQILRSM |
| | KGLSST [SEQ ID NO: 59] |
| | (GenPept dbjBAB60865.1) |
| HeV F | ISSQISSMNQSLQQSKDYIKEAQKILDTVNPS |
| | [SEQ ID NO: 60] |
| | (GenPept NP_047111.2) |
| Inf A | RIQDLEKYVEDTKIDLWSYNAELVLALENQHTID |
| HA | LTDSEMSKLFERTRR [SEQ ID NO: 61] |
| | (GenPept gbAEC23340.1] |
| Inf B | DEILELDEKVDDLRADTISSQIELAVLLSNEGII |
| HA | NSEDEHLLALERKLKKML [SEQ ID NO: 62] |
| | (GenPept gbAFH57854.1] |
| EBOV | HDWTKNITDKIDQIIHDFVDKTL |
| GP | [SEQ ID NO: 63] |
| | (GenPept NP_066246.1] |
| MARV | IGIEDLSKNISEQIDQI |
| GP | [SEQ ID NO: 64] |
| | (GenPept YP_001531156.1] |
| MERS S | NFGSLTQINTTLLDLTYEMLSLQQVVKALNESYI |
| | DLKELGNYTY [SEQ ID NO: 65] |
| | (GenPept gbAHX00711.1] |
| SARS S | DVDLGDISGINASVVNIQKEIDRLNEVAKNLNES |
| | LIDLQELGK [SEQ ID NO: 66] |
| | (GenPept gbAAR86788.1] |

The HR1 and HR2 regions are capable of coming together to form an oligomer, typically a hexamer composed of three HR1 regions and three HR2 regions, which is thermodynamically stable and typifies the post-fusion conformation of class I viral fusion proteins. HR1 and HR2 regions with a strong propensity to oligomerize are referred to herein as “complementary” heptad repeat regions. Non-limiting examples of such heptad repeat regions those listed in Table 7.

In particular embodiments, the structure-stabilizing moiety, including one or both of the heptad repeat regions, includes an immune-silencing or suppressing moiety that inhibits elicitation or production of an immune response to the structure-stabilizing moiety, particularly when folded into an anti-parallel, two-helix bundle. These embodiments are advantageous as they can permit the generation of a stronger or enhanced immune response to the ectodomain polypeptide or complex thereof. The immune-silencing moiety can be a glycosylation site that is specifically recognized and glycosylated by a glycosylation enzyme, in particular a glycosyltransferase. Glycosylations can be N-linked or O-linked. N-linked refers to the attachment of the carbohydrate moiety to the side chain of an asparagine residue. The tripeptide sequences N-X-S and N-X-T, where X is any amino acid except P, are the recognition sequences for enzymatic attachment of the carbohydrate moiety to the asparagine side chain, and these sequences are commonly referred to as ‘glycosylation sites’. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The immune-silencing moiety may be inserted into the structure stabilizing moiety, including one or both of the heptad repeat regions.

In other embodiments, unnatural or nonnative amino acids can be incorporated into one or both of the heptad repeat regions using an expanded genetic code. The nonnative amino acids are biosynthetically incorporated into a desired location using tyrosyl-tRNA/aminoacyl-tRNA synthetase orthogonal pair and a nonsense codon at the desired site. The nonnative or unnatural amino acids are supplied to cells expressing a construct from which the chimeric polypeptide is expressible, from an external source and this strategy can incorporate side chains with a wide range of physical attributes including, but not limited to, chemical crosslinking group (e.g., azide or haloalkane), a trackable maker (e.g., fluorescent or radioactive) and photosensitive groups to enable temporally controlled modifications. To these unnatural amino acids various moieties can be covalently linked by chemical addition to the structure-stabilizing moiety to provide advantageous properties.

TABLE 7
COMPLEMENTARY HEPTAD REPEAT REGION SEQUENCES

| VIRUS | HR1 REGION SEQUENCE | HR2 REGION SEQUENCE |
|---|---|---|
| Synthetic | IEEIQKQIAAIQKQIAAIQKQIYRM | IEEIQKQIAAIQKQIAAIQKQIYRM |
| | [SEQ ID NO: 42] | [SEQ ID NO: 42] |
| HIV | SGIVQQQNNLLRAIEAQQHLLQLTVWGIKQLQA | HTTWMEWDREINNYTSLIHSLIEESQNQQEKNE |
| GP160 | RILA [SEQ ID NO: 43] | QELLE [SEQ ID NO: 55] (GenPept |
| | (GenPept dbjBAF31430.1) | dbjBAF31430.1) |
| RSV F | LHLEGEVNKIKSALLSTNKAVVSLGNGVSVLTS | FDASISQVNEKINQSLAFIRKSDELLHNVNAGK |
| | KVLDLK [SEQ ID NO: 44] | STTN [SEQ ID NO: 56] (GenPept |
| | (GenPept gbAHL84194.1] | gbAHL84194.1] |
| hMPV F | IRLESEVTAIKNALKKTNEAVSTLGNGVRVLAT | FNVALDQVFESIENSQALVDQSNRILSSAEKGN |
| | AVRELK [SEQ ID NO: 45] | TG [SEQ ID NO: 57] (GenPept |
| | (GenPept gbAAN52913.1] | gbAAN52913.1) |
| PIV F | KQARSDIEKLKEAIRDTNKAVQSVQSSIGNLIV | IELNKAKSDLEESKEWIRRSNQKLDSIGNWHQS |
| | AIKSVQ [SEQ ID NO: 46] | STT [SEQ ID NO: 58] (GenPept |
| | (GenPept gbAAB21447.1) | gbAAB21447.1] |
| MeV F | MLNSQAIDNLRASLETTNQAIEAIRQAGQEMIL | LERLDVGTNLGNAIAKLEDAKELLESSDQILRS |
| | AVQGVQ [SEQ ID NO: 47] | MKGLSST [SEQ ID NO: 59] (GenPept |
| | (dbjBAB60865.1) | dbjBAB60865.1) |
| HeV F | MKNADNINKLKSSIESTNEAVVKLQETAEKTVY | ISSQISSMNQSLQQSKDYIKEAQKILDTVNPS |
| | VLTALQ [SEQ ID NO: 48] | [SEQ ID NO: 60] (GenPept |
| | (GenPept NP_047111.2) | NP_047111.2] |
| Inf A HA | ENGWEGMVDGWYGFRHQNSEGTGQAADLKSTQA | RIQDLEKYVEDTKIDLWSYNAELVLALENQHTI |
| | AI [SEQ ID NO: 49] | DLTDSEMSKLFERTRR [SEQ ID NO: 61] |
| | (GenPept gbAEC23340.1) | (GenPept gbAEC23340.1) |
| Inf B HA | HGYTSHGAHGVAVAADLKSTQEAINKITKNLNY | DEILELDEKVDDLRADTISSQIELAVLLSNEGI |
| | L [SEQ ID NO: 50] (GenPept | INSEDEHLLALERKLKKML [SEQ ID |
| | gbAFH57854.1) | NO: 62] (GenPept gbAFH57854.1) |
| EBOV GP | GLRQLANETTQALQLFLRATTELRTFSILNRKA | HDWTKNITDKIDQIIHDFVDKTL [SEQ ID |
| | IDFL [SEQ ID NO: 51] (GenPept | NO: 63] (GenPept NP_066246.1) |
| | NP_066246.1) | |
| MARV GP | LANQTAKSLELLLRVTTEERTFSLINRHAIDFL | IGIEDLSKNISEQIDQI [SEQ ID NO: 64] |
| | LTRW [SEQ ID NO: 52] (GenPept | (GenPept YP_001531156.1) |
| | YP_001531156.1) | |
| MERS S | ISASIGDIIQRLDVLEQDAQIDRLINGRLTTLN | GSGGNFGSLTQINTTLLDLTYEMLSLQQVVKAL |
| | AFVAQQLVRSESAALSAQLAKDKVNEG [SEQ | NESYIDLKELGNYTY [SEQ ID NO: 65] |
| | ID NO: 53] (GenPept gbAHX00711.1) | (GenPept gbAHX00711.1) |
| SARS S | ISSVLNDILSRLDKVEAEVQIDRLITGRLQSLQ | GSGGDVDLGDISGINASVVNIQKEIDRLNEVAK |
| | TYVTQQLIRAAEIRASANLAATKMSEG [SEQ | NLNESLIDLQELGK [SEQ ID NO: 66] |
| | ID NO: 54] (GenPept gbAAR86788.1) | (GenPept gbAAR86788.1) |

Further embodiments may include any possible combination of the above examples, or additional unnatural chemical addition, covalently linked to the structure-stabilizing moiety.

Optionally, one or more additional cysteine residues may be inserted into the HR1 and/or HR2 regions to form disulfide bonds and further stabilize the anti-parallel, α-helical coiled coil structure of the structure stabilizing moiety.

Linkers for Spacing Heptad-Repeat Regions

The structure-stabilizing moiety of the present disclosure suitably comprises a linker that spaces the heptad repeat regions (also referred to herein as HR1 and HR2). The linker generally includes any amino acid residue that cannot be unambiguously assigned to a heptad repeat sequence. Linkers are frequently used in the field of protein engineering to interconnect different functional units, e.g., in the creation of single-chain variable fragment (scFv) constructs derived from antibody variable light (VL) and variable heavy (VH) chains. They are generally conformationally flexible in solution, and are suitably and predominantly composed of polar amino acid residue types. Typical (frequently used) amino acids in flexible linkers are serine and glycine. Less preferably, flexible linkers may also include alanine, threonine and proline. Thus, an intervening linker of the structure-stabilizing moiety is preferably flexible in conformation to ensure relaxed (unhindered) association of HR1 and HR2 as two-helix bundle that suitably adopts an α-helical coiled coil structure. Suitable linkers for use in the polypeptides envisaged herein will be clear to the skilled person, and may generally be any linker used in the art to link amino acid sequences, as long as the linkers are structurally flexible, in the sense that they permit, and suitably do not impair, assembly of the characteristic two-helix bundle structure of the structure-stabilizing moiety.

The skilled person will be able to determine the optimal linkers, optionally after performing a limited number of routine experiments. The intervening linker is suitably an amino acid sequence generally consisting of at least 1 amino acid residue and usually consisting of at least 2 amino acid residues, with a non-critical upper limit chosen for reasons of convenience being about 100 amino acid residues. In particular embodiments, the linker consists of about 1 to about 50 amino acid residues, or about 50 to about 100 amino acid residues, usually about 1 to about 40 amino acid residues, typically about 1 to about 30 amino acid residues. In non-limiting examples, the linker has about the same number of amino acids as the number of amino acids connecting complementary HRA and HRB regions of a Class I enveloped virus fusion protein. In particular, non-limiting embodiments, at least 50% of the amino acid residues of a linker sequence are selected from the group proline, glycine, and serine. In further non-limiting embodiments, at least 60%, such as at least 70%, such as for example 80% and more particularly 90% of the amino acid residues of a linker sequence are selected from the group proline, glycine, and serine. In other particular embodiments, the linker sequences essentially consist of polar amino acid residues; in such particular embodiments, at least 50%, such as at least 60%, such as for example 70% or 80% and more particularly 90% or up to 100% of the amino acid residues of a linker sequence are selected from the group consisting of glycine, serine, threonine, alanine, proline, histidine, asparagine, aspartic acid, glutamine, glutamic acid, lysine and arginine. In specific embodiments, linker sequences may include [GGSG]ₙGG, [GGGGS]ₙ, [GGGGG]ₙ, [GGGKGGGG]ₙ, [GGGNGGGG]ₙ, [GGGCGGGG]ₙ, wherein n is an integer from 1 to 10, suitably 1 to 5, more suitably 1 to 3.

In specific embodiments in which the heptad repeat regions comprise, consist or consist essentially of complementary HRA and HRB regions, respectively, of a Class I enveloped virus fusion protein, the linker comprises, consists or consists essentially of an intervening naturally-occurring amino acid sequence, which connects the HRA and HRB regions. The intervening sequence can be full-length, or about full-length or can comprise, consist or consist essentially of one or more portions of a full-length intervening naturally-occurring amino acid sequence. In other embodiments, the linker lacks a naturally-occurring amino acid sequence interposed between the HRA and HRB regions of a wild-type Class I enveloped virus fusion protein. In any of the above embodiments, the linker may comprise one or more non-naturally-occurring amino acid sequences.

In addition to spacing the heptad repeat regions of the structure-stabilizing moiety and preferably introducing structural flexibility to facilitate anti-parallel association of those regions, the linker may comprise one or more ancillary functionalities. For example, the linker may comprise a purification moiety that facilitates purification of the chimeric polypeptide and/or at least one immune-modulating moiety that modulates an immune response to the chimeric polypeptide.

Purification moieties typically comprise a stretch of amino acids that enables recovery of the chimeric polypeptide through affinity binding. Numerous purification moieties or ‘tags’ are known in the art, illustrative examples of which include biotin carboxyl carrier protein-tag (BCCP-tag), Myc-tag (c-myc-tag), Calmodulin-tag, FLAG-tag, HA-tag, His-tag (Hexahistidine-tag, His6, 6H), Maltose binding protein-tag (MBP-tag), Nus-tag, Chitin-binding protein-tag (CBP-tag) Glutathione-S-transferase-tag (GST-tag), Green fluorescent protein-tag (GFP-tag), Polyglutamate-tag, Amyloid beta-tag, Thioredoxin-tag, S-tag, Softag 1, Softag 3, Strep-tag, Streptavidin-binding peptide-tag (SBP-tag), biotin-tag, streptavidin-tag and V5-tag.

Immune-modulating moieties can be introduced into the linker to modulate the immune response elicited by the chimeric polypeptide or complex thereof. Non-limiting examples of such moieties include immune-silencing or suppressing moieties as described for example above, antigenic moieties, including antigenic moieties derived from pathogenic organisms, or other disease associated antigenic moieties such as cancer or tumor associated antigens. Exemplary pathogenic organisms include, but are not limited to, viruses, bacteria, fungi parasites, algae and protozoa and amoebae. In specific embodiments, the antigenic moieties are derived from antigens of pathogenic viruses. Illustrative viruses responsible for diseases including, but not limited to, measles, mumps, rubella, poliomyelitis, hepatitis A, B (e.g., GenBank Accession No. E02707), and C (e.g., GenBank Accession No. E06890), as well as other hepatitis viruses, influenza, adenovirus (e.g., types 4 and 7), rabies (e.g., GenBank Accession No. M34678), yellow fever, Epstein-Barr virus and other herpesviruses such as papillomavirus, Ebola virus, influenza virus, Japanese encephalitis (e.g., GenBank Accession No. E07883), dengue (e.g., GenBank Accession No. M24444), hantavirus, Sendai virus, respiratory syncytial virus, orthomyxoviruses, vesicular stomatitis virus, visna virus, cytomegalovirus and human immunodeficiency virus (HIV) (e.g., GenBank Accession No. U18552). Any suitable antigen derived from such viruses are useful in the practice of the present disclosure. For example, illustrative retroviral antigens derived from HIV include, but are not limited to, antigens such as gene products of the gag, pol, and env genes, the Nef protein, reverse transcriptase, and other HIV components. Illustrative examples of hepatitis viral antigens include, but are not limited to, antigens such as the S, M, and L proteins of hepatitis B virus, the pre-S antigen of hepatitis B virus, and other hepatitis, e.g., hepatitis A, B, and C. Illustrative examples of influenza viral antigens include; but are not limited to, antigens such as hemagglutinin and neuraminidase and other influenza viral components. Illustrative examples of measles viral antigens include, but are not limited to, antigens such as the measles virus fusion protein and other measles virus components. Illustrative examples of rubella viral antigens include, but are not limited to, antigens such as proteins E1 and E2 and other rubella virus components; rotaviral antigens such as VP7sc and other rotaviral components. Illustrative examples of Cytomegaloviral antigens include, but are not limited to, antigens such as envelope glycoprotein B and other Cytomegaloviral antigen components. Non-limiting examples of respiratory syncytial viral antigens include antigens such as the RSV fusion protein, the M2 protein and other respiratory syncytial viral antigen components. Illustrative examples of herpes simplex viral antigens include, but are not limited to, antigens such as immediate early proteins, glycoprotein D, and other herpes simplex viral antigen components. Non-limiting examples of varicella zoster viral antigens include antigens such as 9PI, gpII, and other varicella zoster viral antigen components. Non-limiting examples of Japanese encephalitis viral antigens include antigens such as proteins E, M-E, M-E-NS 1, NS 1, NS 1-NS2A, 80% E, and other Japanese encephalitis viral antigen components. Representative examples of rabies viral antigens include, but are not limited to, antigens such as rabies glycoprotein, rabies nucleoprotein and other rabies viral antigen components. Illustrative examples of papillomavirus antigens include, but are not limited to, the L1 and L2 capsid proteins as well as the E6/E7 antigens associated with cervical cancers, See Fundamental Virology, Second Edition, eds. Fields, B. N. and Knipe, D. M., 1991, Raven Press, New York, for additional examples of viral antigens. In particular embodiments, the viral antigen is an antigen of an enveloped virus to which the ectodomain polypeptide corresponds. In other embodiments, the viral antigen is an antigen of a different enveloped virus to which the ectodomain polypeptide corresponds.

In some embodiments, one or more cancer- or tumor-associated antigens are inserted into the linker. Such antigens include, but are not limited to, MAGE-2, MAGE-3, MUC-1, MUC-2, HER-2, high molecular weight melanoma-associated antigen MAA, GD2, carcinoembryonic antigen (CEA), TAG-72, ovarian-associated antigens OV-TL3 and MOV 18, TUAN, alpha-feto protein (AFP), OFP, CA-125, CA-50, CA-19-9, renal tumor-associated antigen G250, EGP-40 (also known as EpCAM), S100 (malignant melanoma-associated antigen), p53, prostate tumor-associated antigens (e.g., PSA and PSMA), p21ras, Her2/neu, EGFR, EpCAM, VEGFR, FGFR, MUC-I, CA 125, CEA, MAGE, CD20, CD19, CD40, CD33, A3, antigen specific to A33 antibodies, BrE3 antigen, CD1, CD1a, CD5, CD8, CD14, CD15, CD16, CD21, CD22, CD23, CD30, CD33, CD37, CD38, CD40, CD45, CD46, CD52, CD54, CD74, CD79a, CD126, CD138, CD154, B7, Ia, Ii, HMI.24, HLA-DR (e.g., HLA-DR10), NCA95, NCA90, HCG and sub-units, CEA (CEACAMS), CEACAM-6, CSAp, EGP-I, EGP-2, Ba 733, KC4 antigen, KS-I antigen, KS1-4, Le-Y, MUC2, MUC3, MUC4, PIGF, ED-B fibronectin, NCA 66a-d, PAM-4 antigen, PSA, PSMA, RS5, SIOO, TAG-72, T101, TAG TRAIL-RI, TRAIL-R2, p53, tenascin, insulin growth factor-1 (IGF-I), Tn antigen etc.

The antigenic moiety or moieties included in the linker may correspond to full-length antigens or part antigens. When part antigens are employed, the part antigens may comprise one or more epitopes of an antigen of interest, including B cell epitopes and/or T cell epitopes (e.g., cytotoxic T lymphocyte (CTL) epitopes and/or T helper (Th) epitopes). Epitopes of numerous antigens are known in the literature or can be determined using routine techniques known to persons of skill in the art. In other embodiments the linker may include another cell targeting moiety which can provide delivery to a specific cell type within the immunized individual. Cell populations of interest include, but are not limited to, B-cells, Microfold cells and antigen-presenting cells (APC). In the later example the targeting moiety facilitates enhanced recognition of the chimeric polypeptide or complex thereof to an APC such as a dendritic cell or macrophage. Such targeting sequences can enhance APC presentation of epitopes of an associated ectodomain polypeptide, which can in turn augment the resultant immune response, including intensification or broadening the specificity of either or both of antibody and cellular immune responses to the ectodomain polypeptide. Non-limiting examples of APC-targeting moieties include ligands that bind to APC surface receptors such as, but not limited to, mannose-specific lectin (mannose receptor), IgG Fc receptors, DC-SIGN, BDCA3 (CD141), 33D1, SIGLEC-H, DCIR, CD11c, heat shock protein receptors and scavenger receptors. In particular embodiments, the APC-targeting moiety is a dendritic cell targeting moiety, which comprises, consists or consists essentially of the sequence FYPSYHSTPQRP (Uriel, et al., J. Immunol. 2004 172: 7425-7431) or NWYLPWLGTNDW (Sioud, et al., FASEB J 2013 27(8): 3272-83).

2.1.2 Representative Chimeric Polypeptides

Non-limiting examples of chimeric polypeptides in accordance with the present disclosure are provided below.

SARS-CoV-2 Spike Protein-Q⁶⁷⁵-GSG-S⁶⁹¹-G¹²⁰⁴-HIV GP160-Based SSM

[SEQ ID NO: 67]
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQGSGSIIAYTMS
LGAENSVAYSNNSIAIPTNFTISVTTEILPVSMTKTSVDCTMYICGDST
ECSNLLLQYGSFCTQLNRALTGIAVEQDKNTQEVFAQVKQIYKTPPIKD
FGGFNFSQILPDPSKPSKRSFIEDLLFNKVTLADAGFIKQYGDCLGDIA
ARDLICAQKFNGLTVLPPLLTDEMIAQYTSALLAGTITSGWTFGAGAAL
QIPFAMQMAYRFNGIGVTQNVLYENQKLIANQFNSAIGKIQDSLSSTAS
ALGKLQDVVNQNAQALNTLVKQLSSNFGAISSVLNDILSRLDKVEAEVQ
IDRLITGRLQSLQTYVTQQLIRAAEIRASANLAATKMSECVLGQSKRVD
FCGKGYHLMSFPQSAPHGVVFLHVTYVPAQEKNFTTAPAICHDGKAHFP
REGVFVSNGTHWFVTQRNFYEPQIITTDNTFVSGNCDVVIGIVNNTVYD
PLQPELDSFKEELDKYFKNHTSPDVDLGDISGINASVVNIQKEIDRLNE
VAKNLNESLIDLQELGSGIVQQQNNLLRAIEAQQHLLQLTVWGIKQLQA
RILAGGSGGHTTWMEWDREINNYTSLIHSLIEESQNQQEKNEQELLE,

or an amino acid sequence corresponding thereto (e.g., an amino acid sequence having at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98% or 99% sequence similarity or identity);
wherein:
   Italicized text corresponds to the signal peptide of SARS-CoV-2 spike protein;
   Bold text is a flexible linker; and
   Underlined text corresponds to an HIV GP160-based SSM.

SARS-CoV-2 Spike Protein-Q⁶⁷⁵-GGSGG-S⁶⁹¹-G¹²⁰⁴-HIV GP160-Based SSM

[SEQ ID NO: 68]
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQGGSGGSIIAYT
MSLGAENSVAYSNNSIAIPTNFTISVTTEILPVSMTKTSVDCTMYICGD
STECSNLLLQYGSFCTQLNRALTGIAVEQDKNTQEVFAQVKQIYKTPPI
KDFGGFNFSQILPDPSKPSKRSFIEDLLFNKVTLADAGFIKQYGDCLGD
IAARDLICAQKFNGLTVLPPLLTDEMIAQYTSALLAGTITSGWTFGAGA
ALQIPFAMQMAYRFNGIGVTQNVLYENQKLIANQFNSAIGKIQDSLSST
ASALGKLQDVVNQNAQALNTLVKQLSSNFGAISSVLNDILSRLDKVEAE
VQIDRLITGRLQSLQTYVTQQLIRAAEIRASANLAATKMSECVLGQSKR
VDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTV
YDPLQPELDSFKEELDKYFKNHTSPDVDLGDISGINASVVNIQKEIDRL
NEVAKNLNESLIDLQELGSGIVQQQNNLLRAIEAQQHLLQLTVWGIKQL
QARILAGGSGGHTTWMEWDREINNYTSLIHSLIEESQNQQEKNEQELLE,

or an amino acid sequence corresponding thereto (e.g., an amino acid sequence having at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98% or 99% sequence similarity or identity);
wherein:
   Italicized text corresponds to the signal peptide of SARS-CoV-2 spike protein;
   Bold text is a flexible linker; and
   Underlined text corresponds to an HIV GP160-based SSM.

SARS-CoV-2 Spike Protein-Q⁶⁷⁷-GSG-S⁶⁸⁹-G¹²⁰⁴ HIV GP160-Based SSM

[SEQ ID NO: 69]
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQGSGSQSIIA
YTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTP
PIKDFGGFNFSQILPDPSKPSKRSFIEDLLFNKVTLADAGFIKQYGDCL
GDIAARDLICAQKFNGLTVLPPLLTDEMIAQYTSALLAGTITSGWTFGA
GAALQIPFAMQMAYRFNGIGVTQNVLYENQKLIANQFNSAIGKIQDSLS
STASALGKLQDVVNQNAQALNTLVKQLSSNFGAISSVLNDILSRLDKVE
AEVQIDRLITGRLQSLQTYVTQQLIRAAEIRASANLAATKMSECVLGQS
KRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNN
TVYDPLQPELDSFKEELDKYFKNHTSPDVDLGDISGINASVVNIQKEID
RLNEVAKNLNESLIDLQELGSGIVQQQNNLLRAIEAQQHLLQLTVWGIK
QLQARILAGGSGGHTTWMEWDREINNYTSLIHSLIEESQNQQEKNEQEL
LE,

or an amino acid sequence corresponding thereto (e.g., an amino acid sequence having at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98% or 99% sequence similarity or identity);
wherein:
   Italicized text corresponds to the signal peptide of SARS-CoV-2 spike protein;
   Bold text is a flexible linker; and
   Underlined text corresponds to an HIV GP160-based SSM.

SARS-CoV-2 Spike Protein-Q⁶⁷⁷-GGSGG-S⁶⁸⁹-G¹²⁰⁴-HIV GP160-Based SSM

[SEQ ID NO: 70]
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQGGSGGSQSI
IAYTMSLGAENSVAYSNNSIAIPTNFTISVTTEILPVSMTKTSVDCTMY
ICGDSTECSNLLLQYGSFCTQLNRALTGIAVEQDKNTQEVFAQVKQIYK
TPPIKDFGGFNFSQILPDPSKPSKRSFIEDLLFNKVTLADAGFIKQYGD
CLGDIAARDLICAQKFNGLTVLPPLLTDEMIAQYTSALLAGTITSGWTF
GAGAALQIPFAMQMAYRFNGIGVTQNVLYENQKLIANQFNSAIGKIQDS
LSSTASALGKLQDVVNQNAQALNTLVKQLSSNFGAISSVLNDILSRLDK
VEAEVQIDRLITGRLQSLQTYVTQQLIRAAEIRASANLAATKMSECVLG
QSKRVDFCGKGYHLMSFPQSAPHGVVFLHVTYVPAQEKNFTTAPAICHD
GKAHFPREGVFVSNGTHWFVTQRNFYEPQIITTDNTFVSGNCDVVIGIV
NNTVYDPLQPELDSFKEELDKYFKNHTSPDVDLGDISGINASVVNIQKE
IDRLNEVAKNLNESLIDLQELGSGIVQQQNNLLRAIEAQQHLLQLTVWG
IKQLQARILAGGSGGHTTWMEWDREINNYTSLIHSLIEESQNQQEKNEQ
ELLE,

or an amino acid sequence corresponding thereto (e.g., an amino acid sequence having at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98% or 99% sequence similarity or identity);
wherein:
   Italicized text corresponds to the signal peptide of SARS-CoV-2 spike protein;
   Bold text is a flexible linker; and
   Underlined text corresponds to an HIV GP160-based SSM.

SARS-CoV-2 Spike Protein-N⁶⁷⁹-GSG-S⁶⁹¹-G¹²⁰⁴ HIV GP160-Based SSM

[SEQ ID NO: 71]
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNGSGSIIA
YTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTP
PIKDFGGFNFSQILPDPSKPSKRSFIEDLLFNKVTLADAGFIKQYGDCL
GDIAARDLICAQKFNGLTVLPPLLTDEMIAQYTSALLAGTITSGWTFGA
GAALQIPFAMQMAYRFNGIGVTQNVLYENQKLIANQFNSAIGKIQDSLS
STASALGKLQDVVNQNAQALNTLVKQLSSNFGAISSVLNDILSRLDKVE
AEVQIDRLITGRLQSLQTYVTQQLIRAAEIRASANLAATKMSECVLGQS
KRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNN
TVYDPLQPELDSFKEELDKYFKNHTSPDVDLGDISGINASVVNIQKEID
RLNEVAKNLNESLIDLQELGSGIVQQQNNLLRAIEAQQHLLQLTVWGIK
QLQARILAGGSGGHTTWMEWDREINNYTSLIHSLIEESQNQQEKNEQEL
LE,

or an amino acid sequence corresponding thereto (e.g., an amino acid sequence having at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98% or 99% sequence similarity or identity);
wherein:
   Italicized text corresponds to the signal peptide of SARS-CoV-2 spike protein;
   Bold text is a flexible linker; and
   Underlined text corresponds to an HIV GP160-based SSM.

SARS-CoV-2 Spike Protein-N⁶⁷⁹-GGSGG-S⁶⁹¹-G¹²⁰⁴ HIV GP160-Based SSM

[SEQ ID NO: 72]
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNGGSGGSI
IAYTMSLGAENSVAYSNNSIAIPTNFTISVTTEILPVSMTKTSVDCTMY
ICGDSTECSNLLLQYGSFCTQLNRALTGIAVEQDKNTQEVFAQVKQIYK
TPPIKDFGGFNFSQILPDPSKPSKRSFIEDLLFNKVTLADAGFIKQYGD
CLGDIAARDLICAQKFNGLTVLPPLLTDEMIAQYTSALLAGTITSGWTF
GAGAALQIPFAMQMAYRFNGIGVTQNVLYENQKLIANQFNSAIGKIQDS
LSSTASALGKLQDVVNQNAQALNTLVKQLSSNFGAISSVLNDILSRLDK
VEAEVQIDRLITGRLQSLQTYVTQQLIRAAEIRASANLAATKMSECVLG
QSKRVDFCGKGYHLMSFPQSAPHGVVFLHVTYVPAQEKNFTTAPAICHD
GKAHFPREGVFVSNGTHWFVTQRNFYEPQIITTDNTFVSGNCDVVIGIV
NNTVYDPLQPELDSFKEELDKYFKNHTSPDVDLGDISGINASVVNIQKE
IDRLNEVAKNLNESLIDLQELGSGIVQQQNNLLRAIEAQQHLLQLTVWG
IKQLQARILAGGSGGHTTWMEWDREINNYTSLIHSLIEESQNQQEKNEQ
ELLE,

or an amino acid sequence corresponding thereto (e.g., an amino acid sequence having at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98% or 99% sequence similarity or identity);
wherein:
   Italicized text corresponds to the signal peptide of SARS-CoV-2 spike protein;
   Bold text is a flexible linker; and
   Underlined text corresponds to an HIV GP160-based SSM.

SARS-CoV-2 Spike Protein-P⁶⁸¹-GSG-S⁶⁸⁶-G¹²⁰⁴-HIV GP160-Based SSM

[SEQ ID NO: 73]
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPGSGSV
ASQSIIAYTMSLGAENSVAYSNNSIAIPTNFTISVTTEILPVSMTKTSV
DCTMYICGDSTECSNLLLQYGSFCTQLNRALTGIAVEQDKNTQEVFAQV
KQIYKTPPIKDFGGFNFSQILPDPSKPSKRSFIEDLLFNKVTLADAGFI
KQYGDCLGDIAARDLICAQKFNGLTVLPPLLTDEMIAQYTSALLAGTIT
SGWTFGAGAALQIPFAMQMAYRFNGIGVTQNVLYENQKLIANQFNSAIG
KIQDSLSSTASALGKLQDVVNQNAQALNTLVKQLSSNFGAISSVLNDIL
SRLDKVEAEVQIDRLITGRLQSLQTYVTQQLIRAAEIRASANLAATKMS
ECVLGQSKRVDFCGKGYHLMSFPQSAPHGVVFLHVTYVPAQEKNFTTAP
AICHDGKAHFPREGVFVSNGTHWFVTQRNFYEPQIITTDNTFVSGNCDV
VIGIVNNTVYDPLQPELDSFKEELDKYFKNHTSPDVDLGDISGINASVV
NIQKEIDRLNEVAKNLNESLIDLQELGSGIVQQQNNLLRAIEAQQHLLQ
LTVWGIKQLQARILAGGSGGHTTWMEWDREINNYTSLIHSLIEESQNQQ
EKNEQELLE,

or an amino acid sequence corresponding thereto (e.g., an amino acid sequence having at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98% or 99% sequence similarity or identity);
wherein:
   Italicized text corresponds to the signal peptide of SARS-CoV-2 spike protein;
   Bold text is a flexible linker; and
   Underlined text corresponds to an HIV GP160-based SSM.

SARS-CoV-2 Spike Protein-P⁶⁸¹-GGSGG-S⁶⁹¹-G¹²⁰⁴ HIV GP160-Based SSM

[SEQ ID NO: 74]
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPGGSGG
SVASQSIIAYTMSLGAENSVAYSNNSIAIPTNFTISVTTEILPVSMTKT
SVDCTMYICGDSTECSNLLLQYGSFCTQLNRALTGIAVEQDKNTQEVFA
QVKQIYKTPPIKDFGGFNFSQILPDPSKPSKRSFIEDLLFNKVTLADAG
FIKQYGDCLGDIAARDLICAQKFNGLTVLPPLLTDEMIAQYTSALLAGT
ITSGWTFGAGAALQIPFAMQMAYRFNGIGVTQNVLYENQKLIANQFNSA
IGKIQDSLSSTASALGKLQDVVNQNAQALNTLVKQLSSNFGAISSVLND
ILSRLDKVEAEVQIDRLITGRLQSLQTYVTQQLIRAAEIRASANLAATK
MSECVLGQSKRVDFCGKGYHLMSFPQSAPHGVVFLHVTYVPAQEKNFTT
APAICHDGKAHFPREGVFVSNGTHWFVTQRNFYEPQIITTDNTFVSGNC
DVVIGIVNNTVYDPLQPELDSFKEELDKYFKNHTSPDVDLGDISGINAS
VVNIQKEIDRLNEVAKNLNESLIDLQELGSGIVQQQNNLLRAIEAQQHL
LQLTVWGIKQLQARILAGGSGGHTTWMEWDREINNYTSLIHSLIEESQN
QQEKNEQELLE,

or an amino acid sequence corresponding thereto (e.g., an amino acid sequence having at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98% or 99% sequence similarity or identity);
wherein:
   Italicized text corresponds to the signal peptide of SARS-CoV-2 spike protein;
   Bold text is a flexible linker; and
   Underlined text corresponds to an HIV GP160-based SSM.

SARS-CoV-2 Spike Protein-N⁶⁷⁹-GSG-S⁶⁹¹-G¹²⁰⁴-Immunosilenced HIV GP160-Based SSM

[SEQ ID NO: 75]
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNGSGSIIA
YTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTP
PIKDFGGFNFSQILPDPSKPSKRSFIEDLLFNKVTLADAGFIKQYGDCL
GDIAARDLICAQKFNGLTVLPPLLTDEMIAQYTSALLAGTITSGWTFGA
GAALQIPFAMQMAYRFNGIGVTQNVLYENQKLIANQFNSAIGKIQDSLS
STASALGKLQDVVNQNAQALNTLVKQLSSNFGAISSVLNDILSRLDKVE
AEVQIDRLITGRLQSLQTYVTQQLIRAAEIRASANLAATKMSECVLGQS
KRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNN
TVYDPLQPELDSFKEELDKYFKNHTSPDVDLGDISGINASVVNIQKEID
RLNEVAKNLNESLIDLQELGSGIVQQQNNLLRAIEAQQHLLQLTVWGIK
QLQARILAGGSGGNHTTWMNWSREINNYTSLIHNLTEESQNQTEKNEQE
LLE,

or an amino acid sequence corresponding thereto (e.g., an amino acid sequence having at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98% or 99% sequence similarity or identity),
wherein:
   Italicized text corresponds to the signal peptide of SARS-CoV-2 spike protein;
   Bold text is a flexible linker;
   Underlined regular text corresponds to an HIV GP160-based SSM; and
   Underlined italicized text correspond to N-linked glycosylation sites.

Further non-limiting examples of chimeric polypeptides in accordance with the present disclosure are provided in Examples 5 and 6. In Example 5 the lead candidate as identified in Example 1 is connected upstream of various alternative viral-derived SSMs instead of the HIV GP160-based SSM as illustrated in Example 1. These various alternative SSMs are derived from RSV F, hMPV F, PIV F, MEV F, HEV F, Inf A HA, Inf B HA, EBOV GP, MARV GP, MERS S and SARS S. The primary sequences of the SARS-CoV-2 Sclamp viral-derived SSM constructs with these alternative SSMs are shown in SEQ ID NOs 87 to 97. SEQ ID NOs 87 to 97 are depicted in Example 5, wherein the viral-derived SSM sequences correspond to the parts of SEQ ID NOs 87 to 97 as shown as italicized non-bolded text. In Example 6 the lead candidate as identified in Example 1 is connected upstream of an SSM derived from the T4-phage (also called “foldon”) instead of the HIV GP160-based SSM as illustrated in Example 1. The T4-derived SSM sequences are SEQ ID NOs 38 and 39. The “foldon” construct is shown in SEQ ID NO: 98.

In preferred embodiments, the chimeric polypeptide comprises, consists or consists essentially of the amino acid sequence set forth in any of SEQ ID NO: 71 or 75 or SEQ ID NOs 87 to 98, or an amino acid sequence corresponding thereto (e.g., an amino acid sequence having at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98% or 99% sequence similarity or identity).

2.2 Methods of Preparing Chimeric Polypeptide Constructs

The modified polypeptides and chimeric polypeptides of the present disclosure may be prepared by chemical synthesis or recombinant means. Usually, the polypeptides are prepared by expression of a recombinant construct that encodes the modified or chimeric polypeptide in suitable host cells, although any suitable methods can be used. Suitable host cells include, for example, insect cells (e.g., Aedes aegypti, Autographa californica, Bombyx mori, Drosophila melanogaster, Spodoptera frugiperda, and Trichoplusia ni), mammalian cells (e.g., human, non-human primate, horse, cow, sheep, dog, cat, and rodent (e.g., hamster), avian cells (e.g., chicken, duck, and geese), bacteria (e.g., Escherichia coli, Bacillus subtilis, and Streptococcus spp.), yeast cells (e.g., Saccharomyces cerevisiae, Candida albicans, Candida maltosa, Hansenula polymorphs, Kluyveromyces fragilis, Kluyveromyces lactis, Pichia guillerimondii, Pichia pastoris, Schizosaccharomyces pombe and Yarrowia lipolytica), Tetrahymena cells (e.g., Tetrahymena thermophile) or combinations thereof. Many suitable insect cells and mammalian cells are well-known in the art. Suitable insect cells include, for example, Sf9 cells, Sf21 cells, Tn5 cells, Schneider S2 cells, and High Five cells (a clonal isolate derived from the parental Trichoplusia ni BTI-TN-5B1-4 cell line (Invitrogen)). Suitable mammalian cells include, for example, Chinese hamster ovary (CHO) cells, human embryonic kidney cells (HEK293 cells, typically transformed by sheared adenovirus type 5 DNA), NIH-3T3 cells, 293-T cells, Vero cells, HeLa cells, PERC.6 cells (ECACC deposit number 96022940), Hep G2 cells, MRC-5 (ATCC CCL-171), WI-38 (ATCC CCL-75), fetal rhesus lung cells (ATCC CL-160), Madin-Darby bovine kidney (“MDBK”) cells, Madin-Darby canine kidney (“MDCK”) cells (e.g., MDCK (NBL2), ATCC CCL34; or MDCK 33016, DSM ACC 2219), baby hamster kidney (BHK) cells, such as BHK21-F, HKCC cells, and the like. Suitable avian cells include, for example, chicken embryonic stem cells (e.g., EBx® cells), chicken embryonic fibroblasts, chicken embryonic germ cells, duck cells (e.g., AGE1.CR and AGE1.CR.pIX cell lines (ProBioGen) which are described, for example, in Vaccine 27:4975-4982 (2009) and WO2005/042728), EB66 cells, and the like.

Appropriate insect cell expression systems, such as Baculovirus systems, are known to those of skill in the art and described in, e.g., Summers and Smith, Texas Agricultural Experiment Station Bulletin No. 1555 (1987). Materials and methods for Baculovirus/insert cell expression systems are commercially available in kit form from, inter alia, Invitrogen, San Diego Calif. Avian cell expression systems are also known to those of skill in the art and described in, e.g., U.S. Pat. Nos. 5,340,740; 5,656,479; 5,830,510; 6,114,168; and 6,500,668; European Patent No. EP 0787180B; European Patent Application No. EP03291813.8; WO 03/043415; and WO 03/076601. Similarly, bacterial and mammalian cell expression systems are also known in the art and described in, e.g., Yeast Genetic Engineering (Barr et al., eds., 1989) Butterworths, London.

Recombinant constructs encoding the modified or chimeric polypeptides of the present disclosure can be prepared in suitable vectors using conventional methods. A number of suitable vectors for expression of recombinant proteins in insect or mammalian cells are well-known and conventional in the art. Suitable vectors can contain a number of components, including, but not limited to one or more of the following: an origin of replication; a selectable marker gene; one or more expression control elements, such as a transcriptional control element (e.g., a promoter, an enhancer, a terminator), and/or one or more translation signals; and a signal sequence or leader sequence for targeting to the secretory pathway in a selected host cell (e.g., of mammalian origin or from a heterologous mammalian or non-mammalian species). For example, for expression in insect cells a suitable Baculovirus expression vector, such as pFastBac (Invitrogen), can be used to produce recombinant Baculovirus particles. The Baculovirus particles are amplified and used to infect insect cells to express recombinant protein. For expression in mammalian cells, a vector that will drive expression of the construct in the desired mammalian host cell (e.g., Chinese hamster ovary cells) is used.

The modified or chimeric polypeptides can be purified using any applicable method. Suitable methods for purifying desired proteins including precipitation and various types of chromatography, such as hydrophobic interaction, ion exchange, affinity, chelating and size exclusion are well-known in the art. Appropriate purification schemes can be created using two or more of these or other suitable methods. If desired, the modified or chimeric polypeptides can include a purification moiety or “tag” that facilitates purification, as described for example supra. Such tagged polypeptides can conveniently be purified, for example from conditioned media, by chelating chromatography or affinity chromatography.

The modified or chimeric polypeptides may include additional sequences. For example, for expression purposes, the natural leader peptide of a heterologous polypeptide of interest (e.g., the natural leader peptide of an enveloped virus fusion protein) may be substituted for a different one.

### 3. Polynucleotides and Nucleic Acid Constructs for Endogenous Production of Chimeric Polypeptides

The present disclosure also contemplates polynucleotides and nucleic acid constructs for endogenous production of modified or chimeric polypeptides in a host organism, suitably a vertebrate animal, preferably a mammal such as a human.

Polynucleotides contemplated herein comprise a coding sequence for the modified polypeptide and chimeric polypeptide of the disclosure. These polynucleotides are useful for making nucleic acid constructs from which a modified polypeptide or chimeric polypeptide coding sequence is expressible for immunizing subjects. In some embodiments, these polynucleotides are themselves useful for immunizing subjects directly. In representative embodiments of this type, the polynucleotides comprise at least one ribonucleic acid (RNA) having an open reading frame encoding a polypeptide of the present disclosure. In some embodiments, the RNA is a messenger RNA (mRNA) having an open reading frame that codes for a polypeptide disclosed herein.

Polynucleotides of the present disclosure, in some embodiments, are codon optimized. Codon optimization methods are known in the art and may be used for optimizing expression of the polypeptides disclosed herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g. glycosylation sites); add, remove or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art. Non-limiting examples include services from GeneArt (Life Technologies), DNA2.0 (Menlo Park Calif.) and/or proprietary methods. In some embodiments, the open reading frame (ORF) sequence is optimized using optimization algorithms. In some embodiments a codon optimized RNA may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

In some embodiments, the RNA polynucleotides of the present disclosure may further comprise sequence comprising or encoding additional sequence, for example, one or more functional domain(s), one or more further regulatory sequence(s), and/or an engineered 5′ cap. Thus, in some embodiments, the RNA vaccines comprise a 5′UTR element, an optionally codon optimized open reading frame, and a 3′UTR element, a poly(A) sequence and/or a polyadenylation signal wherein the RNA is not chemically modified.

The RNA polynucleotide may be transcribed in vitro from template DNA, referred to as an “in vitro transcription template”. In some embodiments, an in vitro transcription template encodes a 5′ untranslated (UTR) region, contains an open reading frame, and encodes a 3′ UTR and a polyA tail. The particular nucleotide sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of codons beginning with a start codon (e.g., methionine (ATG)), and ending with a stop codon (e.g., TAA, TAG or TGA) that encodes a polypeptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30.40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, export of the mRNA from the nucleus and translation.

In some embodiments, the RNA polynucleotide is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-0 methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-0 methyl-transferase. Cap 3 structure may be generated from the Cap 2 structure followed by the 2′-O-methylation of the 5′-preantepenultimate nucleotide using a 2′-0 methyl-transferase. Enzymes may be derived from a recombinant source.

The present disclosure also contemplates nucleic acid constructs for endogenous production of the polypeptides disclosed herein. The nucleic acid constructs can be self-replicating extra-chromosomal vectors/replicons (e.g., plasmids) or vectors that integrate into a host genome. In specific embodiments, the nucleic acid constructs are viral vectors. Exemplary viral vectors include retroviral vectors, lentiviral vectors, poxvirus vectors, vaccinia virus vectors, adenovirus vectors, adenovirus-associated virus vectors, herpes virus vectors, flavivirus vectors, and alphavirus vectors. Viral vectors may be live, attenuated, replication conditional or replication deficient, and typically is a non-pathogenic (defective), replication competent viral vector.

By way of example, when the viral vector is a vaccinia virus vector, a polynucleotide encoding a chimeric polypeptide of the disclosure may be inserted into a non-essential site of a vaccinia viral vector genome. Such non-essential sites are described, for example, in Perkus et al. (1986. Virology 152:285); Hruby et al. (1983. Proc. Natl. Acad. Sci. USA Weir et al. (1983. J. Virol. 46:530). Suitable promoters for use with vaccinia viruses include but are not limited to P7.5 (see, e.g., Cochran et al. 1985. J. Virol. 54:30); P11 (see, e.g., Bertholet, et al., 1985. Proc. Natl. Acad. Sci. USA 82:2096); and CAE-1 (see, e.g., Patel et al., 1988. Proc. Natl. Acad. Sci. USA 85:9431). Highly attenuated strains of vaccinia are more acceptable for use in humans and include Lister, NYVAC, which contains specific genome deletions (see, e.g., Guerra et al., 2006. J. Virol. 80:985-998); Tartaglia et al., 1992. AIDS Research and Human Retroviruses 8:1445-1447), or MVA (see, e.g., Gheradi et al., 2005. J. Gen. Virol. 86:2925-2936); Mayr et al., 1975. Infection 3:6-14). See also Hu et al. (2001. J. Virol. 75:10300-10308), describing use of a Yaba-Like disease virus as a vector for cancer therapy); U.S. Pat. Nos. 5,698,530 and 6,998,252. See also, e.g., U.S. Pat. No. 5,443,964. See also U.S. Pat. Nos. 7,247,615 and 7,368,116.

In certain embodiments, an adenovirus vector may be used for expressing a chimeric polypeptide of interest. The adenovirus on which a viral transfer vector may be based may be from any origin, any subgroup, any subtype, mixture of subtypes, or any serotype. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. Adenoviral serotypes 1 through 51 are available from the American Type Culture Collection (ATCC, Manassas, Va.). Non-group C adenoviruses, and even non-human adenoviruses, can be used to prepare replication-deficient adenoviral vectors. Non-group C adenoviral vectors, methods of producing non-group C adenoviral vectors, and methods of using non-group C adenoviral vectors are disclosed in, for example, U.S. Pat. Nos. 5,801,030, and 5,849,561, and International Patent Applications WO 97/12986 and WO 98/53087. Any adenovirus, even a chimeric adenovirus, can be used as the source of the viral genome for an adenoviral vector. For example, a human adenovirus can be used as the source of the viral genome for a replication-deficient adenoviral vector. Further examples of adenoviral vectors can be found in Molin et al. (1998. J. Virol. 72:8358-8361), Narumi et al. (1998. Am J. Respir. Cell Mol. Biol. 19:936-941) Mercier et al. (2004. Proc. Natl. Acad. Sci. USA 101:6188-6193), U.S. Publication Nos. 20150093831, 20140248305, 20120283318, 20100008889, 20090175897 and 20090088398 and U.S. Pat. Nos. 6,143,290; 6,596,535; 6,855,317; 6,936,257; 7,125,717; 7,378,087; 7,550,296.

The viral vector can also be based on adeno-associated viruses (AAVs). For a description of AAV-based vectors, see, for example, U.S. Pat. Nos. 8,679,837, 8,637,255, 8,409,842, 7,803,622, and 7,790,449, and U.S. Publication Nos. 20150065562, 20140155469, 20140037585, 20130096182, 20120100606, and 20070036757. The AAV vectors may also be self-complementary (sc) AAV vectors, which are described, for example, in U.S. Patent Publications 2007/01110724 and 2004/0029106, and U.S. Pat. Nos. 7,465,583 and 7,186,699.

Herpes simplex virus (HSV)-based viral vectors are also suitable for endogenous production of the chimeric polypeptides of the disclosure. Many replication-deficient HSV vectors contain a deletion to remove one or more intermediate-early genes to prevent replication. Advantages of the herpes vector are its ability to enter a latent stage that can result in long-term DNA expression, and its large viral DNA genome that can accommodate exogenous DNA up to 25 kb. For a description of HSV-based vectors, see, for example, U.S. Pat. Nos. 5,837,532, 5,846,782, 5,849,572, and 5,804,413, and International Patent Applications WO 91/02788, WO 96/04394, WO 98/15637, and WO 99/06583.

Retroviral vectors may include those based upon murine leukemia virus (MuLV), gibbon ape leukemia virus (GaLV), ecotropic retroviruses, simian immunodeficiency virus (SW), human immunodeficiency virus (HIV), and combinations (see, e.g., Buchscher et al., 1992. J. Virol. 66:2731-2739; Johann et al., 1992. J. Virol. 66:1635-1640; Sommerfelt et al., 1990. Virology 176:58-59; Wilson et al., 1989. J. Virol. 63:2374-2378; Miller et al., 1991. J. Virol. 65:2220-2224; Miller et al., 1990. Mol. Cell Biol. 10:4239; Kolberg, 1992. NIH Res. 4:43; Cornetta et al., 1991. Hum. Gene Ther. 2:215).

In specific embodiments, the retroviral vector is a lentiviral vector. As would be understood by the skilled person, a viral vector, such as a lentiviral vector, generally refers to a viral vector particle that comprises the viral vector genome. For example, a lentiviral vector particle may comprise a lentiviral vector genome. With respect to lentiviral vectors, the vector genome can be derived from any of a large number of suitable, available lentiviral genome based vectors, including those identified for human gene therapy applications (see, e.g., Pfeifer et al., 2001. Annu. Rev. Genomics Hum. Genet. 2:177-211). Suitable lentiviral vector genomes include those based on Human Immunodeficiency Virus (HIV-1), HIV-2, feline immunodeficiency virus (FIV), equine infectious anemia virus, Simian Immunodeficiency Virus (SIV), and maedi/visna virus. A desirable characteristic of lentiviruses is that they are able to infect both dividing and non-dividing cells, although target cells need not be dividing cells or be stimulated to divide. Generally, the genome and envelope glycoproteins will be based on different viruses, such that the resulting viral vector particle is pseudotyped. Safety features of the viral vector are desirably incorporated. Safety features include self-inactivating LTR and integration deficiency as described in more detail herein. In certain embodiments integration deficiency may be conferred by elements of the vector genome but may also derive from elements of the packaging system (e.g., a non-functional integrase protein that may not be part of the vector genome but supplied in trans). Exemplary vectors contain a packaging signal (psi), a Rev-responsive element (RRE), splice donor, splice acceptor, optionally a central poly-purine tract (cPPT), and WPRE element. In certain exemplary embodiments, the viral vector genome comprises sequences from a lentivirus genome, such as the HIV-1 genome or the SIV genome. The viral genome construct may comprise sequences from the 5′ and 3′ LTRs of a lentivirus, and in particular may comprise the R and U5 sequences from the 5′ LTR of a lentivirus and an inactivated or self-inactivating 3′ LTR from a lentivirus. The LTR sequences may be LTR sequences from any lentivirus from any species. For example, they may be LTR sequences from HIV, SIV, FIV or BIV. Typically, the LTR sequences are HIV LTR sequences.

The vector genome may comprise an inactivated or self-inactivating 3′ LTR (see, e.g., Zufferey et al., 1998. J. Virol. 72: 9873; Miyoshi et al., 1998. J. Virol. 72:8150). A self-inactivating vector generally has a deletion of the enhancer and promoter sequences from the 3′ long terminal repeat (LTR), which is copied over into the 5′ LTR during vector integration. In one instance, the U3 element of the 3′ LTR contains a deletion of its enhancer sequence, the TATA box, Spl and NF-kappa B sites. As a result of the self-inactivating 3′ LTR, the provirus that is generated following entry and reverse transcription will comprise an inactivated 5′ LTR. The rationale is to improve safety by reducing the risk of mobilization of the vector genome and the influence of the LTR on nearby cellular promoters. The self-inactivating 3′ LTR may be constructed by any method known in the art.

Optionally, the U3 sequence from the lentiviral 5′ LTR may be replaced with a promoter sequence in the viral construct, such as a heterologous promoter sequence. This can increase the titer of virus recovered from the packaging cell line. An enhancer sequence may also be included. Any enhancer/promoter combination that increases expression of the viral RNA genome in the packaging cell line may be used. In one example, the CMV enhancer/promoter sequence is used (see, e.g., U.S. Pat. Nos. 5,385,839 and 5,168,062).

In certain embodiments, the risk of insertional mutagenesis is minimized by constructing the lentiviral vector to be integration defective. A variety of approaches can be pursued to produce a non-integrating vector genome. These approaches entail engineering a mutation(s) into the integrase enzyme component of the pol gene, such that it encodes a protein with an inactive integrase. The vector genome itself can be modified to prevent integration by, for example, mutating or deleting one or both attachment sites, or making the 3′ LTR-proximal polypurine tract (PPT) non-functional through deletion or modification. In addition, non-genetic approaches are available; these include pharmacological agents that inhibit one or more functions of integrase. The approaches are not mutually exclusive, that is, more than one of them can be used at a time. For example, both the integrase and attachment sites can be non-functional, or the integrase and PPT site can be non-functional, or the attachment sites and PPT site can be non-functional, or all of them can be non-functional.

Exemplary lentivirus vectors are described for example in U.S. Publication Nos. 20150224209, 20150203870, 20140335607, 20140248306, 20090148936, and 20080254008.

The viral vectors may also be based on an alphavirus. Alphaviruses include Sindbis virus (and Venezuelan equine encephalitis virus (VEEV)), Aura virus, Babanki virus, Barmah Forest virus, Bebaru virus, Cabassou virus, Chikungunya virus, Eastern equine encephalitis virus, Everglades virus, Fort Morgan virus, Getah virus, Highlands J virus, Kyzylagach virus, Mayaro virus, Me Tri virus, Middelburg virus, Mosso das Pedras virus, Mucambo virus, Ndumu virus, O'nyong-nyong virus, Pixuna virus, Rio Negro virus, Ross River virus, Salmon pancreas disease virus, Semliki Forest virus (SFV), Southern elephant seal virus, Tonate virus, Trocara virus, Una virus, Venezuelan equine encephalitis virus, Western equine encephalitis virus, and Whataroa virus. Generally, the genome of such viruses encode nonstructural (e.g., replicon) and structural proteins (e.g., capsid and envelope) that can be translated in the cytoplasm of the host cell. Ross River virus, Sindbis virus, SFV, and VEEV have all been used to develop viral transfer vectors for transgene delivery. Pseudotyped viruses may be formed by combining alphaviral envelope glycoproteins and retroviral capsids. Examples of alphaviral vectors can be found in U.S. Publication Nos. 20150050243, 20090305344, and 20060177819.

Alternatively, the viral vectors can be based on a flavivirus. Flaviviruses include Japanese encephalitis virus, Dengue virus (e.g., Dengue-1, Dengue-2, Dengue-3, Dengue-4), Yellow fever virus, Murray Valley encephalitis virus, St. Louis encephalitis virus, West Nile virus, Kunjin virus, Rocio encephalitis virus, Ilheus virus, Tick-borne encephalitis virus, Central European encephalitis virus, Siberian encephalitis virus, Russian Spring-Summer encephalitis virus, Kyasanur Forest Disease virus, Omsk Hemorrhagic fever virus, Louping ill virus, Powassan virus, Negishi virus, Absettarov virus, Hansalova virus, Apoi virus, and Hypr virus. Examples of flavivirus vectors can be found in U.S. Publication Nos. 20150231226, 20150024003, 20140271708, 20140044684, 20130243812, 20120294889, 20120128713, 20110135686, 20110014229, 20110003884, 20100297167, 20100184832, 20060159704, 20060088937, 20030194801 and 20030044773.

### 4. Chimeric Polypeptide Complexes

The chimeric polypeptides of the present disclosure can self-assemble under suitable conditions to form chimeric polypeptide complexes. Accordingly, the present disclosure further encompasses a method of producing a chimeric polypeptide complex, wherein the method comprises: combining chimeric polypeptides of the present disclosure under conditions (e.g., in aqueous solution) suitable for the formation of a chimeric polypeptide complex, whereby a chimeric polypeptide complex is produced that comprises three chimeric polypeptides. In specific embodiments in which and is characterized by a six-helix bundle formed by the coiled coil structures of the respective structure-forming moieties of the chimeric polypeptides. The chimeric polypeptides that are combined may be identical or non-identical to thereby form homotrimers and heterotrimers, respectively.

Generally the chimeric polypeptides self-assemble in a buffered aqueous solution (e.g., pH about 5 to about 9). If required, mild denaturing conditions can be used, such as, by including urea, small amounts of organic solvents or heat to mildly denature the chimeric polypeptides in order to facilitate refolding and self-assembly.

Any suitable preparation of chimeric polypeptides can be used in the method. For example, conditioned cell culture media that contains the desired chimeric polypeptide can be used in the method. However, it is preferable to use purified chimeric polypeptides in the method.

In particular embodiments in which a structure-stabilizing moiety comprising complementary first and second heptad repeat regions is used to oligomerize modified polypeptides to form chimeric polypeptide complexes, the modified polypeptide subunits of the complexes are in the pre-fusion conformation. Consistent with the disclosure of International Application Publication No. WO 2018/176103, it is believed that the pre-fusion form of the modified polypeptide trimer is stabilized in the complexes described herein because the heterologous structure-stabilizing moiety induces complex formation and prevents internal moieties or domains of the ectodomain polypeptide (e.g., the HRA and HRB regions of the SARS-CoV-2 spike protein) from interacting. The interaction of such internal moieties or domains leads to refolding into the post fusion form.

### 5. Antigen-Binding Molecules

The modified polypeptides, chimeric polypeptides and complexes of the present disclosure are useful for producing antigen-binding molecules, which are preferably proteins (i.e., “antigen-binding protein”) that are immuno-interactive with a SARS-CoV-2 spike protein. In specific embodiments, the modified polypeptides, chimeric polypeptides and complexes include at least one pre-fusion epitope that is not present in the post-fusion form of the SARS-CoV-2 spike protein, and therefore useful for preparation of antigen-binding molecules that are immuno-interactive with a metastable or pre-fusion form of the SARS-CoV-2 spike protein.

Those of ordinary skill in the art will appreciate the well-developed knowledge base on antigen-binding proteins such as set forth, for example, in Abbas et al., Cellular and Molecular Immunology, 6^{th} ed., W.B. Saunders Company (2010) or Murphey et al., Janeway's Immunobiology, 8^{th} ed., Garland Science (2011), each of which is incorporated herein by reference in its entirety.

In some embodiments, antigen binding proteins that are immuno-interactive with the modified polypeptides, chimeric polypeptides and complexes of the present disclosure are antibodies. Antibodies include intact antibodies and antigen binding fragments thereof, as described in the definition section. An antibody may comprise a complete antibody molecule (including polyclonal, monoclonal, chimeric, humanized, or human versions having full length heavy and/or light chains), or comprise an antigen binding fragment thereof. Antibody fragments include F(ab′)₂, Fab, Fab′, Fv, Fc, and Fd fragments, and can be incorporated into single domain antibodies, single-chain antibodies, maxibodies, minibodies, intrabodies, diabodies, triabodies, tetrabodies, v-NAR and bis-scFv (see e.g., Hollinger and Hudson, 2005, Nature Biotechnology, 23, 9, 1126-1136). Also included are antibody polypeptides such as those disclosed in U.S. Pat. No. 6,703,199, including fibronectin polypeptide monobodies. Other antibody polypeptides are disclosed in U.S. Patent Publication 2005/0238646, which are single-chain polypeptides.

Numerous methods of preparing antibodies to antigens of interest are known in the art. For example, monoclonal antibodies that bind specifically with the modified polypeptides, chimeric polypeptides and complexes can be made using conventional hybridoma methods that are often based on the seminal method of Kohler, G. et al. (1975, “Continuous Cultures Of Fused Cells Secreting Antibody Of Predefined Specificity,” Nature 256:495-497) or a modification thereof. Typically, monoclonal antibodies are developed in non-human species, such as mice. In general, a mouse or rat is used for immunization but other animals may also be used. The antibodies may be produced by immunizing mice with an immunogenic amount of an immunogen, in this case a modified polypeptide, chimeric polypeptide or complex of the present disclosure. The immunogen may be administered multiple times at periodic intervals such as, bi weekly, or weekly, or may be administered in such a way as to maintain viability in the animal.

To monitor the antibody response, a small biological sample (e.g., blood) may be obtained from the animal and tested for antibody titer against the immunogen. The spleen and/or several large lymph nodes can be removed and dissociated into single cells. If desired, the spleen cells may be screened (after removal of non-specifically adherent cells) by applying a cell suspension to a plate or to a well coated with the antigen. 6-cells, expressing membrane-bound immunoglobulin specific for the antigen, will bind to the plate, and are not rinsed away with the rest of the suspension. Resulting 6-cells, or all dissociated spleen cells, can then be fused with myeloma cells (e.g., X63-Ag8.653 and those from the Salk Institute, Cell Distribution Center, San Diego, Calif.). Polyethylene glycol (PEG) may be used to fuse spleen or lymphocytes with myeloma cells to form a hybridoma. The hybridoma is then cultured in a selective medium (e.g., hypoxanthine, aminopterin, thymidine medium, otherwise known as “HAT medium”). The resulting hybridomas are then plated by limiting dilution, and are assayed for the production of antibodies that bind specifically to the immunogen, using, for example, FACS (fluorescence activated cell sorting) or immunohistochemistry (IHC) screening. The selected monoclonal antibody-secreting hybridomas are then cultured either in vitro (e.g., in tissue culture bottles or hollow fiber reactors), or in vivo (e.g., as ascites in mice).

As another alternative to the cell fusion technique, Epstein-Barr Virus (EBV)-immortalized B cells may be used to produce monoclonal antibodies that bind specifically with a modified polypeptide, chimeric polypeptide or complex of the present disclosure. The hybridomas are expanded and subcloned, if desired, and supernatants are assayed for anti-immunogen activity by conventional assay procedures (e.g., FACS, IHC, radioimmunoassay, enzyme immunoassay, fluorescence immunoassay, etc.).

Thus, the present disclosure further contemplates methods of producing an antigen-binding molecule that binds specifically with a modified polypeptide, chimeric polypeptide or complex as described herein, wherein the method comprises: (1) immunizing an animal with a a modified polypeptide, chimeric polypeptide or complex of the present disclosure; (2) detecting a B cell from the animal, which binds specifically with the modified polypeptide, chimeric polypeptide or complex or coronavirus spike protein; and (3) isolating the antigen-binding molecule expressed by that B cell.

The present disclosure also encompasses antigen-binding molecule that are produced by such methods as well as derivatives thereof. Also encompassed are cells including hybridomas that are capable of producing the antigen-binding molecules of the disclosure, and methods of producing antigen-binding molecules from those cells. In specific embodiments, the antigen-binding molecules produced by the methods and cells of the disclosure are preferably neutralizing antigen-binding molecules.

Also contemplated are chimeric antibodies and humanized antibodies. In some embodiments, a humanized monoclonal antibody comprises the variable domain of a murine antibody (or all or part of the antigen binding site thereof) and a constant domain derived from a human antibody. Alternatively, a humanized antibody fragment may comprise the antigen binding site of a murine monoclonal antibody and a variable domain fragment (lacking the antigen-binding site) derived from a human antibody. Procedures for the production of engineered monoclonal antibodies include those described in Riechmann et al., 1988, Nature 332:323, Liu et al., 1987, Proc. Nat. Acad. Sci. USA 84:3439, Larrick et al., 1989, Bio/Technology 7:934, and Winter et al., 1993, TIPS 14:139. In one embodiment, the chimeric antibody is a CDR grafted antibody. Techniques for humanizing antibodies are discussed in, e.g., U.S. Pat. Nos. 5,869,619; 5,225,539; 5,859,205; 6,881,557, Padlan et al., 1995, FASEB J. 9:133-39, Tamura et al., 2000, J. Immunol. 164:1432-41, Zhang, W., et al., Molecular Immunology 42(12):1445-1451, 2005; Hwang W. et al., Methods 36(1):35-42, 2005; Dall'Acqua W F, et al., Methods 36(1):43-60, 2005; and Clark, M., Immunology Today 21(8):397-402, 2000.

An antibody of the present disclosure may also be a fully human monoclonal antibody. Fully human monoclonal antibodies may be generated by any number of techniques with which those having ordinary skill in the art will be familiar. Such methods include, but are not limited to, Epstein Barr Virus (EBV) transformation of human peripheral blood cells (e.g., containing B lymphocytes), in vitro immunization of human B-cells, fusion of spleen cells from immunized transgenic mice carrying inserted human immunoglobulin genes, isolation from human immunoglobulin V region phage libraries, or other procedures as known in the art and based on the disclosure herein.

Procedures have been developed for generating human monoclonal antibodies in non-human animals. For example, mice in which one or more endogenous immunoglobulin genes have been inactivated by various means have been prepared. Human immunoglobulin genes have been introduced into the mice to replace the inactivated mouse genes. In this technique, elements of the human heavy and light chain locus are introduced into strains of mice derived from embryonic stem cell lines that contain targeted disruptions of the endogenous heavy chain and light chain loci (see also Bruggemann et al., Curr. Opin. Biotechnol. 8:455-58 (1997)). For example, human immunoglobulin transgenes may be mini-gene constructs, or transloci on yeast artificial chromosomes, which undergo B-cell-specific DNA rearrangement and hypermutation in the mouse lymphoid tissue.

Antibodies produced in the animal incorporate human immunoglobulin polypeptide chains encoded by the human genetic material introduced into the animal. In one embodiment, a non-human animal, such as a transgenic mouse, is immunized with a subject modified polypeptide, chimeric polypeptide or complex immunogen.

Examples of techniques for production and use of transgenic animals for the production of human or partially human antibodies are described in U.S. Pat. Nos. 5,814,318, and 5,545,806, Davis et al., Production of human antibodies from transgenic mice in Lo, ed. Antibody Engineering: Methods and Protocols, Humana Press, NJ:191-200 (2003), Kellermann et al., Curr Opin Biotechnol. 2002, 13:593-97, Russel et al., Infect Immun. 2000, 68:1820-26, Gallo et al., Eur J. Immun. 2000, 30:534-40, Davis et al., Cancer Metastasis Rev. 1999, 18:421-25, Green, J Immunol Methods 1999, 231:11-23, Jakobovits, Advanced Drug Delivery Reviews 1998, 31:33-42, Green et al., J Exp Med. 1998, 188:483-95, Jakobovits A, Exp. Opin. Invest. Drugs 1998, 7:607-14, Tsuda et al., Genomics 1997, 42:413-21, Mendez et al., Nat. Genet. 1997, 15:146-56, Jakobovits, Curr Biol. 1994, 4:761-63, Arbones et al., Immunity 1994, 1:247-60, Green et al., Nat. Genet. 1994, 7:13-21, Jakobovits et al., Nature 1993, 362:255-58, Jakobovits et al., Proc Natl Acad Sci USA 1993, 90:2551-55. Chen, J., M. et al. Int. Immunol. 1993, 5: 647-656, Choi et al., Nature Genetics 1993, 4: 117-23, Fishwild et al., Nature Biotech. 1996, 14: 845-51, Harding et al., 1995, Annals of the New York Academy of Sciences, Lonberg et al., 1994, Nature 368: 856-59, Lonberg, 1994, Transgenic Approaches to Human Monoclonal Antibodies in Handbook of Experimental Pharmacology 113: 49-101, Lonberg et al., Int. Rev. Immunol. 1995, 13: 65-93, Neuberger, Nature Biotech. 1996, 14: 826, Taylor et al., Nucleic Acids Research 1992, 20: 6287-95, Taylor et al., Int. Immunol. 1994, 6: 579-91, Tomizuka et al., Nature Genetics 1997, 16: 133-43, Tomizuka et al., Proc Natl Acad Sci USA 2000, 97: 722-27, Tuaillon et al., Proc Natl Acad Sci USA 1993, 90: 3720-24, and Tuaillon et al., J. Immunol. 1994, 152: 2912-20.; Lonberg et al., Nature 1994, 368:856; Taylor et al., Int. Immunol. 1994, 6:579; U.S. Pat. No. 5,877,397; Bruggemann et al., Curr. Opin. Biotechnol. 1997 8:455-58; Jakobovits et al., Ann. N.Y. Acad. Sci. 1995. 764:525-35. In addition, protocols involving the XenoMouse®. (Abgenix, now Amgen, Inc.) are described, for example in U.S. Ser. No. 05/011,8643 and WO 05/694879, WO 98/24838, WO 00/76310, and U.S. Pat. No. 7,064,244.

Alternatively, the modified polypeptide, chimeric polypeptide or complex disclosed herein may be used to screen for antigen-binding molecules from antigen-binding molecule libraries. For example, a modified polypeptide, chimeric polypeptide or complex of the present disclosure may be immobilized to a solid support (e.g., a silica gel, a resin, a derivatized plastic film, a glass bead, cotton, a plastic bead, a polystyrene bead, an alumina gel, or a polysaccharide, a magnetic bead), and screened for binding to antigen-binding molecules. As an alternative, the antigen-binding molecules may be immobilized to a solid support and screened for binding to the modified polypeptide, chimeric polypeptide or complex. Any screening assay, such as a panning assay, ELISA, surface plasmon resonance, or other antigen-binding molecule screening assay known in the art may be used to screen for antigen-binding molecules that bind to a modified polypeptide, chimeric polypeptide or complex disclosed herein. The antigen-binding molecule library screened may be a commercially available library, an in vitro generated library, or a library obtained by identifying and cloning or isolating antibodies from an individual infected with SARS-CoV-2. In particular embodiments, the antigen-binding molecule library is generated from a survivor of a SARS-CoV-2 outbreak. Antigen-binding molecule libraries may be generated in accordance with methods known in the art. In a particular embodiment, the library is generated by cloning the antibodies and using them in phage display libraries or a phagemid display library.

The present disclosure further encompasses fragments of an anti-modified polypeptide, anti-chimeric polypeptide or anti-complex antibody. Such fragments can consist entirely of antibody-derived sequences or can comprise additional sequences. Examples of antigen-binding fragments include Fab, F(ab′)2, single chain antibodies, diabodies, triabodies, tetrabodies, and domain antibodies. Other examples are provided in Lunde et al., Biochem. Soc. Trans. 2002, 30:500-06.

Single chain antibodies may be formed by linking heavy and light chain variable domain (Fv region) fragments via an amino acid bridge (short peptide linker), resulting in a single polypeptide chain. Such single-chain Fvs (scFvs) have been prepared by fusing DNA encoding a peptide linker between DNAs encoding the two variable domain polypeptides (VL and VH). The resulting polypeptides can fold back on themselves to form antigen-binding monomers, or they can form multimers (e.g., dimers, trimers, or tetramers), depending on the length of a flexible linker between the two variable domains (Kortt et al., Prot. Eng. 1997, 10:423; Kortt et al., Biomol. Eng. 2001, 18:95-108). By combining different VL and VH-comprising polypeptides, one can form multimeric scFvs that bind to different epitopes (Kriangkum et al., Biomol. Eng. 2001, 18:31-40). Techniques developed for the production of single chain antibodies include those described in U.S. Pat. No. 4,946,778; Bird, Science 1988, 242:423; Huston et al., Proc. Natl. Acad. Sci. USA 1988, Ward et al., Nature 1989, 334:544, de Graaf et al., Methods Mol. Biol. 2002, 178:379-87.

Antigen binding fragments derived from an antibody can also be obtained, for example, by proteolytic hydrolysis of the antibody, for example, pepsin or papain digestion of whole antibodies according to conventional methods. By way of example, antibody fragments can be produced by enzymatic cleavage of antibodies with pepsin to provide a 5S fragment termed F(ab′)2. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using papain produces two monovalent Fab fragments and an Fc fragment directly. These methods are described, for example, by Goldenberg, U.S. Pat. No. 4,331,647, Nisonoff et al., Arch. Biochem. Biophys. 1960, 89:230; Porter, Biochem. J. 1959, 73:119; Edelman et al., in Methods in Enzymology 1:422 (Academic Press 1967); and by Andrews, S. M. and Titus, J. A. in Current Protocols in Immunology (Coligan J. E., et al., eds), John Wiley & Sons, New York (2003), pages 2.8.1-2.8.10 and 2.10A.1-2.10A.5. Other methods for cleaving antibodies, such as separating heavy chains to form monovalent light-heavy chain fragments (Fd), further cleaving of fragments, or other enzymatic, chemical, or genetic techniques may also be used, so long as the fragments bind to the antigen that is recognized by the intact antibody.

Another form of an antibody fragment is a peptide comprising one or more complementarity determining regions (CDRs) of an antibody. CDRs can be obtained by constructing polynucleotides that encode the CDR of interest. Such polynucleotides are prepared, for example, by using the polymerase chain reaction to synthesize the variable region using mRNA of antibody-producing cells as a template (see, for example, Larrick et al., Methods: A Companion to Methods in Enzymology 2:106, 1991; Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995); and Ward et al., “Genetic Manipulation and Expression of Antibodies,” in Monoclonal Antibodies: Principles and Applications, Birch et al., (eds.), page 137 (Wiley-Liss, Inc. 1995)). The antibody fragment further may comprise at least one variable region domain of an antibody described herein. Thus, for example, the V region domain may be monomeric and be a VL and VH domain, which is capable of independently binding a subject ectodomain polypeptide or complex with an affinity at least equal to 10⁻⁷ M or less.

The variable region domain may be any naturally occurring variable domain or an engineered version thereof. By engineered version is meant a variable region domain that has been created using recombinant DNA engineering techniques. Such engineered versions include those created, for example, from a specific antibody variable region by insertions, deletions, or changes in or to the amino acid sequences of the specific antibody. Particular examples include engineered variable region domains containing at least one CDR and optionally one or more framework amino acids from a first antibody and the remainder of the variable region domain from a second antibody.

The variable region domain may be covalently attached at a C-terminal amino acid to at least one other antibody domain or a fragment thereof. Thus, for example, a VH domain that is present in the variable region domain may be linked to an immunoglobulin CH1 domain, or a fragment thereof. Similarly a VL domain may be linked to a CK domain or a fragment thereof. In this way, for example, the antibody may be a Fab fragment wherein the antigen binding domain contains associated VH and VL domains covalently linked at their C-termini to a CH1 and CK domain, respectively. The CH1 domain may be extended with further amino acids, for example to provide a hinge region or a portion of a hinge region domain as found in a Fab′ fragment, or to provide further domains, such as antibody CH2 and CH3 domains.

Antigen-binding molecules identified in the methods described herein may be tested for neutralizing activity and lack of autoreactivity using biological assays known in the art or described herein. In some embodiments, an antibody isolated from a non-human animal or an antigen-binding molecule library neutralizes a spike protein from more than one ACE2-interacting coronavirus or ACE2-interactive coronavirus strain. In some embodiments, an antigen-binding molecule elicited or identified using a modified polypeptide, chimeric polypeptide or complex disclosed herein, neutralizes an ACE2-interactive coronavirus selected from SARS-CoV, SARS-CoV-2 and MERS.

In some embodiments, antibodies elicited or identified using a modified polypeptide, chimeric polypeptide or complex disclosed herein may be used to monitor the efficacy of a therapy and/or disease progression.

Antigen-binding molecules elicited or identified using a modified polypeptide, chimeric polypeptide or complex may be used in diagnostic immunoassays to detect the presence of an ACE2-interacting coronavirus in biological samples, passive immunotherapy, and generation of antiidiotypic antigen-binding molecules. In addition, the ability of the antigen-binding molecules to neutralize ACE2-interacting coronavirus spike protein and the specificity of the antigen-binding molecules for the spike protein may be tested prior to using the antibodies in passive immunotherapy.

Specific binding of an antigen-binding molecule to a modified polypeptide, chimeric polypeptide or complex disclosed herein and cross-reactivity with other antigens can be assessed by any method known in the art. Immunoassays which can be used to analyze specific binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds., 1994, Current Protocols in Molecular Biology, Vol. 1, John Wiley & Sons, Inc., New York).

In some embodiments, the antigen-binding molecules disclosed herein are used in immunodetection methods for binding, purifying, removing, quantifying and otherwise generally detecting ACE2-interacting coronavirus, particularly SARS-CoV-2. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The immunodetection methods also include methods for detecting and quantifying the amount of ACE2-interacting coronavirus or related components (e.g., spike protein thereof) in a sample and the detection and quantification of any immune complexes formed during the binding process. In non-limiting examples, a sample suspected of containing ACE2-interacting coronavirus is obtained from a patient, and the sample is contacted with an antigen-binding molecule that binds specifically to a modified polypeptide, chimeric polypeptide or complex disclosed herein, followed by detecting and quantifying the amount of immune complexes formed under the specific conditions. In terms of antigen detection, the biological sample analyzed may be any sample that is suspected of containing an ACE2-interacting coronavirus, such as a tissue section or specimen, a homogenized tissue extract, a biological fluid, including a biological fluid and/or tissue obtained or derived from the respiratory tract including mouth, nose, throat and lungs.

Contacting the chosen biological sample with the antigen-binding molecule under suitable conditions and for a period of time sufficient to allow the formation of immune complexes (primary immune complexes) is generally a matter of simply adding the antigen-binding molecule to the sample and incubating the mixture for a period of time long enough for the antigen-binding molecule to form immune complexes with, i.e., to bind to ACE2-interacting coronavirus or related components (e.g., spike protein thereof) present in the sample. After this time, the sample-antigen-binding molecule composition, such as a tissue section, ELISA plate, dot blot or Western blot, will generally be washed to remove any non-specifically bound antigen-binding molecule species, allowing only those antigen-binding molecule specifically bound within the primary immune complexes to be detected.

### 6. Compositions

The present disclosure further provides compositions, including pharmaceutical compositions, comprising a modified polypeptide, chimeric polypeptide or complex disclosed herein, or a modified polypeptide- or chimeric polypeptide-encoding polynucleotide, or nucleic acid construct from which the modified polypeptide, chimeric polypeptide or complex is expressible. Representative compositions may include a buffer, which is selected according to the desired use of the chimeric polypeptide or complex, and may also include other substances appropriate to the intended use. Where the intended use is to induce an immune response, the composition is referred to as an “immunogenic” or “immunomodulating” composition. Such compositions include preventative compositions (i.e., compositions administered for the purpose of preventing a condition such as an infection) and therapeutic compositions (i.e., compositions administered for the purpose of treating conditions such as an infection). An immunomodulating composition of the present disclosure may therefore be administered to a recipient for prophylactic, ameliorative, palliative, or therapeutic purposes.

Those skilled in the art can readily select an appropriate buffer, a wide variety of which are known in the art, suitable for an intended use. In some instances, the composition can comprise a pharmaceutically acceptable excipient, a variety of which are known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7.sup.th ed., Lippincott, Williams, & Wilkins; and Handbook of Pharmaceutical Excipients (2000) A. H. Kibbe et al., eds., 3.sup.rd ed. Amer. Pharmaceutical Assoc.

In some embodiments, the compositions comprise more than one (i.e., different) modified polypeptide, chimeric polypeptide or complex of the disclosure (e.g., modified or chimeric polypeptides which correspond to the spike proteins of different strains of SARS-CoV-2), one or more modified polypeptide- or chimeric polypeptide-encoding polynucleotides, or one or more nucleic acid constructs from which the modified polypeptide(s), chimeric polypeptide(s) or complex(es) is/are expressible.

Pharmaceutical compositions of the present disclosure may be in a form suitable for administration by injection, in a formulation suitable for oral ingestion (such as, for example, capsules, tablets, caplets, elixirs), in the form of an ointment, cream or lotion suitable for topical administration, in a form suitable for delivery as an eye drop, in an aerosol form suitable for administration by inhalation, such as by intranasal inhalation or oral inhalation, or in a form suitable for parenteral administration, that is, subcutaneous, intramuscular or intravenous injection.

Supplementary active ingredients such as adjuvants or biological response modifiers can also be incorporated into pharmaceutical compositions of the present disclosure. Although adjuvant(s) may be included in pharmaceutical compositions of the present disclosure they need not necessarily comprise an adjuvant. In such cases, reactogenicity problems arising from the use of adjuvants may be avoided.

In general, adjuvant activity in the context of a pharmaceutical composition of the present disclosure includes, but is not limited to, an ability to enhance the immune response (quantitatively or qualitatively) induced by immunogenic components in the composition (e.g., a chimeric polypeptide or complex of the present disclosure). This may reduce the dose or level of the immunogenic components required to produce an immune response and/or reduce the number or the frequency of immunizations required to produce the desired immune response.

Any suitable adjuvant may be included in a pharmaceutical composition of the present disclosure. Such an adjuvant may be selected from any adjuvant known to a skilled person and suitable for the present case, i.e., supporting the induction of an immune response in a mammal. Exemplary adjuvants may be selected from the group consisting of, without being limited thereto, TDM, MDP, muramyl dipeptide, pluronics, alum solution, aluminium hydroxide, ADJUMER™ (polyphosphazene); aluminium phosphate gel; glucans from algae; algammulin; aluminium hydroxide gel (alum); highly protein-adsorbing aluminium hydroxide gel; low viscosity aluminium hydroxide gel; AF or SPT (emulsion of squalane (5%), Tween 80 (0.2%), Pluronic L121 (1.25%), phosphate-buffered saline, pH 7.4); AVRIDINE™ (propanediamine); BAY R1005TM UN-(2-deoxy-2-L-leucylamino-b-D-glucopyranosyl)-N-octadecyl-dodecanoyl-amide hydroacetate); CALCITRIOL™ (1-alpha,25-dihydroxy-vitamin D3); calcium phosphate gel; CAP™ (calcium phosphate nanoparticles); cholera holotoxin, cholera-toxin-A1-protein-A-D-fragment fusion protein, sub-unit B of the cholera toxin; CRL 1005 (block copolymer P1205); cytokine-containing liposomes; DDA (dimethyldioctadecylammonium bromide); DHEA (dehydroepiandrosterone); DMPC (dimyristoylphosphatidylcholine); DMPG (dimyristoylphosphatidylglycerol); DOC/alum complex (deoxycholic acid sodium salt); Freund's complete adjuvant; Freund's incomplete adjuvant; gamma inulin; Gerbu adjuvant (mixture of: i)N-acetylglucosaminyl-(P1-4)-N-acetylmuramyl-L-alanyl-D-glutamine (GMDP), ii) dimethyldioctadecylammonium chloride (DDA), iii) zinc-L-proline salt complex (ZnPro-8); GM-CSF); GMDP (N-acetylglucosaminyl-(b1-4)-N-acetylmuramyl-L-alanyl-D-isoglutamine); imiquimod (1-(2-methypropyl)-1H-imidazo[4,5-c]quinoline-4-amine); ImmTher™ (N-acetylglucosaminyl-N-acetylmuramyl-L-Ala-D-isoGlu-L-Ala-glycerol dipalmitate); DRVs (immunoliposomes prepared from dehydration-rehydration vesicles); interferon-gamma; interleukin-1beta; interleukin-2; interleukin-7; interleukin-12; ISCOMS™; ISCOPREP 7.0.3™; liposomes; LOXORIBINE™ (7-allyl-8-oxoguanosine); LT oral adjuvant (E. coli labile enterotoxin-protoxin); microspheres and microparticles of any composition; MF59C.1®; (squalene-water emulsion); MONTANIDE ISA 51TM (purified incomplete Freund's adjuvant); MONTANIDE ISA 720TM (metabolisable oil adjuvant); MPL™ (3-Q-desacyl-4′-monophosphoryl lipid A); MTP-PE and MTP-PE liposomes ((N-acetyl-L-alanyl-D-isoglutaminyl-L-alanine-2-(1,2-dipalmitoyl-sn-glycero-3-(hydroxyphosphoryloxy))-ethylamide, monosodium salt); MURAMETIDE™ (Nac-Mur-L-Ala-D-Gln-OCH3); MURAPALMITINE™ and D-MURAPALMITINE™ (Nac-Mur-L-Thr-D-isoGln-sn-glyceroldipalmitoyl); NAGO (neuraminidase-galactose oxidase); nanospheres or nanoparticles of any composition; NISVs (non-ionic surfactant vesicles); PLEURAN™ (P3-glucan); PLGA, PGA and PLA (homo- and co-polymers of lactic acid and glycolic acid; microspheres/nanospheres); PLURONIC L121™; PMMA (polymethyl methacrylate); PODDS™ (proteinoid microspheres); polyethylene carbamate derivatives; poly-rA: poly-rU (polyadenylic acid-polyuridylic acid complex); polysorbate 80 (Tween 80); protein cochleates (Avanti Polar Lipids, Inc., Alabaster, Ala.); STIMULON™ (QS-21); Quil-A (Quil-A saponin); S-28463 (4-amino-otec-dimethyl-2-ethoxymethyl-1H-imidazo[4,5 c]quinoline-1-ethanol); SAF-1™ (“Syntex adjuvant formulation”); Sendai proteoliposomes and Sendai-containing lipid matrices; Span-85 (sorbitan trioleate); Specol (emulsion of Marcol 52, Span 85 and Tween 85); squalene or Robane® (2,6,10,15,19,23-hexamethyltetracosan and 2,6,10,15,19,23-hexamethyl-2,6,10,14,18,22-tetracosahexane); stearyltyrosine (octadecyltyrosine hydrochloride); Theramid® (N-acetylglucosaminyl-N-acetylnnurannyl-L-Ala-D-isoGlu-L-Ala-dipalmitoxypro-pylamide); Theronyl-MDP (Termurtide™ or [thr 1]-MDP; N-acetylmuramyl-L-threonyl-D-isoglutamine); Ty particles (Ty-VLPs or virus-like particles); Walter-Reed liposomes (liposomes containing lipid A adsorbed on aluminium hydroxide), and lipopeptides, including Pam3Cys, in particular aluminium salts, such as Adju-phos, Alhydrogel, Rehydragel; emulsions, including CFA, SAF, IFA, Provax, TiterMax, Montanide, Vaxfectin; copolymers, including Optivax (CRL1005), L121, Poloaxmer4010), etc.; liposomes, including Stealth, cochleates, including BIORAL; plant derived adjuvants, including QS21, Quil A, Iscomatrix, ISCOM; adjuvants suitable for costimulation including Tomatine, biopolymers, including PLG, PMM, Inulin; microbe derived adjuvants, including Romurtide, DETOX, MPL, CWS, Mannose, CpG nucleic acid sequences, CpG7909, ligands of human TLR 1-10, ligands of murine TLR 1-13, ISS-1018, IC31, Imidazoquinolines, Ampligen, Ribi529, IMOxine, IRIVs, VLPs, cholera toxin, heat-labile toxin, Pam3Cys, Flagellin, GPI anchor, LNFPIII/Lewis X, antimicrobial peptides, UC-1V150, RSV fusion protein, cdiGMP; and adjuvants suitable as antagonists including CGRP neuropeptide.

Pharmaceutical compositions of the present disclosure may be provided in a kit. The kit may comprise additional components to assist in performing the methods of the present disclosure such as, for example, administration device(s), buffer(s), and/or diluent(s). The kits may include containers for housing the various components and instructions for using the kit components in the methods of the present disclosure.

### 7. Dosages and Routes of Administration

The composition is administered in an “effective amount” that is, an amount effective to achieve an intended purpose in a subject. The dose of active compound(s) administered to a patient should be sufficient to achieve a beneficial response in the subject over time such as a reduction in at least one symptom associated with an infections. The quantity or dose frequency of the pharmaceutically active compounds(s) to be administered may depend on the subject to be treated inclusive of the age, sex, weight and general health condition thereof. In this regard, precise amounts of the active compound(s) for administration will depend on the judgment of the practitioner. One skilled in the art would be able, by routine experimentation, to determine an effective, non-toxic amount of a chimeric polypeptide or complex described herein to include in a pharmaceutical composition of the present disclosure for the desired therapeutic outcome.

In general, a pharmaceutical composition of the present disclosure can be administered in a manner compatible with the route of administration and physical characteristics of the recipient (including health status) and in such a way that it elicits the desired effect(s) (i.e. therapeutically effective, immunogenic and/or protective). For example, the appropriate dosage of a pharmaceutical composition of the present disclosure may depend on a variety of factors including, but not limited to, a subject's physical characteristics (e.g., age, weight, sex), whether the compound is being used as single agent or adjuvant therapy, the type of MHC restriction of the patient, the progression (i.e., pathological state) of a virus infection, and other factors that may be recognized by one skilled in the art. Various general considerations that may be considered when determining an appropriate dosage of a pharmaceutical composition of the present disclosure are described, for example, in Gennaro (2000) “Remington: The Science and Practice of Pharmacy”, edition, Lippincott, Williams, & Wilkins; and Gilman et al., (Eds), (1990), “Goodman And Gilman's: The Pharmacological Bases of Therapeutics”, Pergamon Press.

In some embodiments, an “effective amount” of a subject modified polypeptide, chimeric polypeptide or complex, or a modified polypeptide- or chimeric polypeptide-encoding polynucleotide, or a nucleic acid construct from which the chimeric polypeptide or complex is expressible, is an amount sufficient to achieve a desired prophylactic or therapeutic effect, e.g., to reduce a symptom associated with infection, and/or to reduce the number of infectious agents in the individual. In these embodiments, an effective amount reduces a symptom associated with infection and/or reduces the number of infectious agents in an individual by at least about 10%, at least about 20%, at least about 30%, at least about 40%, at least about 50%, at least about 60%, at least about 70%, at least about 80%, or at least about 90%, or more, when compared to the symptom or number of infectious agents in an individual not treated with the chimeric polypeptide or complex. Symptoms of infection by a pathogenic organism, as well as methods for measuring such symptoms, are known in the art. Methods for measuring the number of pathogenic organisms in an individual are standard in the art.

In some embodiments, an “effective amount” of a subject chimeric polypeptide or complex, or a modified polypeptide- or chimeric polypeptide-encoding polynucleotide, or a nucleic acid construct from which the chimeric polypeptide or complex is expressible, is an amount that is effective in a selected route of administration to elicit an immune response to an ACE2-ineracting coronavirus spike protein, particularly the spike protein of SARS-CoV-2.

In some embodiments, e.g., where the chimeric polypeptide comprises a heterologous antigen, an “effective amount” is an amount that is effective to facilitate elicitation of an immune response against that antigen. For example, where the heterologous antigen is an antigen from a different pathogenic organism than the one from which the ectodomain polypeptide is derived), an “effective amount” of a subject modified polypeptide or chimeric polypeptide or complex, or a modified polypeptide- or chimeric polypeptide-encoding polynucleotide, or a nucleic acid construct from which the chimeric polypeptide or complex is expressible, is an amount that is effective for elicitation of an immune response against that antigen and preferably protection of the host against infection, or symptoms associated with infection, by that pathogenic organism. In these embodiments, an effective amount reduces a symptom associated with infection by the pathogenic organism and/or reduces the number of infectious agents corresponding to the pathogenic organism in an individual by at least about 10%, at least about 20%, at least about 30%, at least about 40%, at least about 50%, at least about 60%, at least about 70%, at least about 80%, or at least about 90%, or more, when compared to the symptom or number of infectious agents in an individual not treated with the modified polypeptide or chimeric polypeptide or complex, or a modified polypeptide- or chimeric polypeptide-encoding polynucleotide, or a nucleic acid construct from which the chimeric polypeptide or complex is expressible. Symptoms of infection by a pathogenic organism, as well as methods for measuring such symptoms, are known in the art.

Alternatively, where a heterologous antigen is a cancer- or tumor-associated antigen, an “effective amount” of a modified polypeptide or chimeric polypeptide or complex, or a modified polypeptide- or chimeric polypeptide-encoding polynucleotide, or a nucleic acid construct from which the chimeric polypeptide or complex is expressible, is an amount that is effective in a route of administration to elicit an immune response effective to reduce or inhibit cancer or tumor cell growth, to reduce cancer or tumor cell mass or cancer or tumor cell numbers, or to reduce the likelihood that a cancer or tumor will form. In these embodiments, an effective amount reduces tumor growth and/or the number of tumor cells in an individual by at least about 10%, at least about 20%, at least about 30%, at least about 40%, at least about 50%, at least about 60%, at least about 70%, at least about 80%, or at least about 90%, or more, when compared to the tumor growth and/or number of tumor cells in an individual not treated with the modified polypeptide or chimeric polypeptide or complex, or a modified polypeptide- or chimeric polypeptide-encoding polynucleotide, or a nucleic acid construct from which the chimeric polypeptide or complex is expressible. Methods of measuring tumor growth and numbers of tumor cells are known in the art.

In various embodiments, the amount of modified polypeptide or chimeric polypeptide or complex, or a modified polypeptide- or chimeric polypeptide-encoding polynucleotide, or a nucleic acid construct from which the chimeric polypeptide or complex is expressible in each dose is selected as an amount that induces an immune response to the encoded ectodomain polypeptide, and/or that induces an immunoprotective or other immunotherapeutic response without significant, adverse side effects generally associated with typical vaccines. Such amount may vary depending upon which specific modified polypeptide or chimeric polypeptide or complex, or a modified polypeptide- or chimeric polypeptide-encoding polynucleotide, or a nucleic acid construct from which the chimeric polypeptide or complex is expressible is employed, whether or not the vaccine formulation comprises an adjuvant, and a variety of host-dependent factors.

A pharmaceutical composition of the present disclosure can be administered to a recipient by standard routes, including, but not limited to, parenteral (e.g., intravenous).

A pharmaceutical composition of the present disclosure may be administered to a recipient in isolation or in conjunction with additional therapeutic agent(s). In embodiments where a pharmaceutical composition is concurrently administered with therapeutic agent(s), the administration may be simultaneous or sequential (i.e., pharmaceutical composition administration followed by administration of the agent(s) or vice versa).

Typically, in treatment applications, the treatment may be for the duration of the disease state or condition. Further, it will be apparent to one of ordinary skill in the art that the optimal quantity and spacing of individual dosages will be determined by the nature and extent of the disease state or condition being treated, the form, route and site of administration, and the nature of the particular individual being treated. Optimum conditions can be determined using conventional techniques.

In many instances (e.g., preventative applications), it may be desirable to have several or multiple administrations of a pharmaceutical composition of the present disclosure. For example, a pharmaceutical composition may be administered 1, 2, 3, 4, 5, 6, 7, 8, 9, 10, or more times. The administrations may be from about one to about twelve week intervals, and in certain embodiments from about one to about four week intervals. Periodic re-administration may be desirable in the case of recurrent exposure to a particular pathogen or other disease-associated component targeted by a pharmaceutical composition of the present disclosure.

It will also be apparent to one of ordinary skill in the art that the optimal course of administration can be ascertained using conventional course of treatment determination tests.

Where two or more entities are administered to a subject “in conjunction” or “concurrently” they may be administered in a single composition at the same time, or in separate compositions at the same time, or in separate compositions separated in time.

Certain embodiments of the present disclosure involve the administration of pharmaceutical compositions in multiple separate doses. Accordingly, the methods for the prevention (i.e. vaccination) and treatment of infection described herein encompass the administration of multiple separated doses to a subject, for example, over a defined period of time. Accordingly, the methods for the prevention (i.e., vaccination) and treatment of infection disclosed herein include administering a priming dose of a pharmaceutical composition of the present disclosure. The priming dose may be followed by a booster dose. The booster may be for the purpose of re-vaccination. In various embodiments, the pharmaceutical composition or vaccine is administered at least once, twice, three times or more.

Methods for measuring the immune response are known to persons of ordinary skill in the art. Exemplary methods include solid-phase heterogeneous assays (e.g., enzyme-linked immunosorbent assay), solution phase assays (e.g., electrochemiluminescence assay), amplified luminescent proximity homogeneous assays, flow cytometry, intracellular cytokine staining, functional T-cell assays, functional B-cell assays, functional monocyte-macrophage assays, dendritic and reticular endothelial cell assays, measurement of NK cell responses, IFN-γ production by immune cells, quantification of virus RNA/DNA in tissues or biological fluids (e.g., quantification of viral RNA or DNA in serum or other fluid or tissue/organ), oxidative burst assays, cytotoxic-specific cell lysis assays, pentamer binding assays, and phagocytosis and apoptosis evaluation.

It will be appreciated by persons skilled in the art that numerous variations and/or modifications may be made to the disclosure as shown in the specific embodiments without departing from the spirit or scope of the present disclosure as broadly described. The present embodiments are, therefore, to be considered in all respects as illustrative and not restrictive.

In order that the disclosure may be readily understood and put into practical effect, particular preferred embodiments will now be described by way of the following non-limiting examples.

### EXAMPLES

### Example 1

### Antigen Design, In Vitro Analysis and Manufacturability

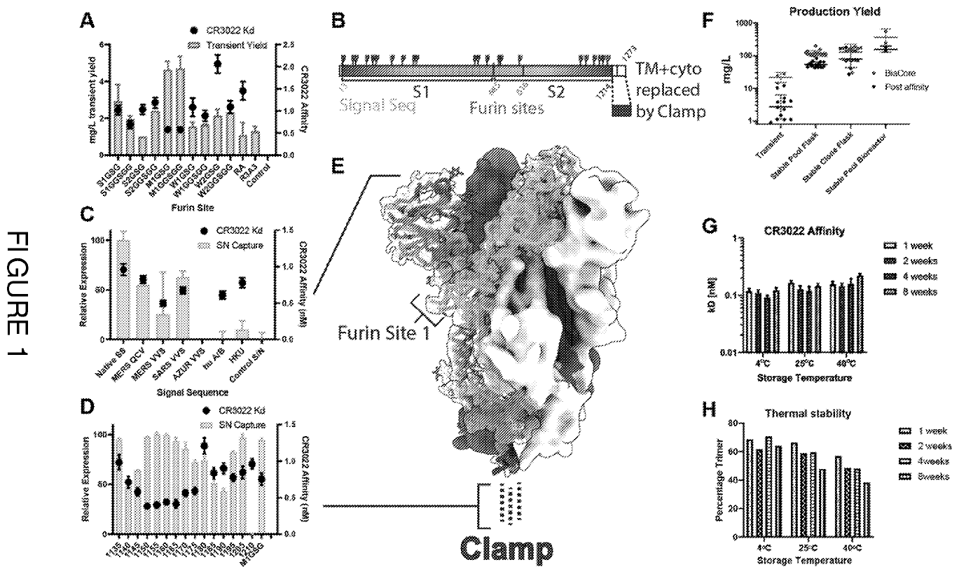

The SARS-CoV-2 genomic sequence first became available on the 12th of January, 2020 ((WHO), W. H. O. Novel Coronavirus (2019-nCoV) Situation Report 1. (https://www.who.int/docs/default-source/coronaviruse/situation-reports/20200121-sitrep-1-2019-ncov.pdf, 2020) and the present inventors commenced development of a candidate subunit vaccine utilizing the molecular clamp platform. Within 34 days of the sequence being released, they had recombinantly expressed and screened more than 200 antigen designs incorporating sequence truncations and modifications at key, generic sites within the spike protein amino acid sequence to identify a lead candidate (FIG. 1).

Briefly, a panel of over 200 candidate subunit vaccines was produced with an HIV GP molecular clamp coding sequence, as described in International Application Publication No. WO 2018/176103, incorporated in place of the transmembrane domain (TM) and cytoplasmic tail of the SARS-CoV-2 spike protein. This panel was produced based on the shared architecture of the SARS-CoV-2 spike protein with other viral class I membrane fusion proteins and the inventors previous knowledge of regions that can impact on yield and homogeneity during the expression of other recombinant viral fusion proteins. Of note, the present inventors identified that the furin cleavage site of SAR-CoV-2 is more similar to the furin cleavage site of the MERS-CoV spike protein than that of the SARS-CoV-1 spike protein. A SARS-CoV-2 homology model was developed using the published ectodomain sequence input for SWISS-MODEL (Waterhouse, A. et al., 2018. Nucleic Acids Res 46 (W1):W296-W303) and the SARS-CoV-1 template model PDB:6ACC. (Song, W. et al. 2018. PLoS Pathog 14(8):e1007236). This full-length ectodomain model was aligned with SARS-CoV-1/MERS-CoV spike protein structure models (Yuan, Y. et al., 2017. Nat. Commun. 8:15092; Walls, A. C. et al., 2019, Cell 176(5):1026-1039.e15) and together with sequence alignment of the S1/S2 furin site for each model, insertion sites were selected for introduction of different linkers, with the aim of minimizing the highly flexible extended loop region around the S1/S2 furin site.

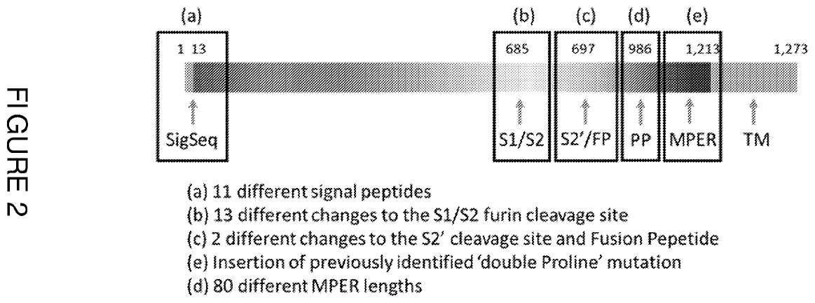

Four structural regions were also identified, which were modified to produce the panel and additionally the previously reported ‘double proline’ mutation shown to facilitate stabilization of other coronavirus spike proteins was also incorporated to some candidates (Wrapp, D. et al., 2020. Science 367(6483):1260-1263; Kirchdoerfer, R. N. et al., 2018. Sci Rep 8(1):15701) (FIG. 2). The sites targeted for modification included: (a) the N-terminal signal peptide, (b) the S1/S2 furin cleavage site, (c) the S2′ proteolytic cleavage site and fusion peptide, (d) The previously identified ‘double proline’ mutation (Wrapp, D. et al., 2020. supra; Kirchdoerfer, R. N. et al., 2018. supra) and (e) the membrane-proximal external region (MPER).

The panel of >200 candidates were screened in vitro for attributes including expression level, presence of the soluble trimeric conformation and reactivity with both the SARS-CoV/SARS-CoV-2 cross-reactive MAb CR3022, as well as the SARS and SARS-CoV-2 in vivo target, soluble recombinant human angiotensin-converting enzyme 2 receptor incorporating a monomeric IgG fragment crystallizable region (hACE2-monoFc) receptor, by enzyme-linked immunosorbent assay (ELISA). Results for several of these candidate vaccines are presented in Table 9 infra.

The lead candidate producing the best results in the screening assays consisted of the trimeric glycosylated SARS-Cov-2 Spike glycoprotein ectodomain fused to HIV GP41 trimerization domain using a 3-amino acid, glycine-serine-glycine linker. Characterization studies of the lead candidate, SARS-CoV-2 Sclamp, exhibited an approximate molecular weight of 600 kDa consistent with a soluble trimeric product, and bound the receptor binding domain specific MAb CR3022 with high affinity (kD=0.30 nM±0.5), as well as the soluble ACE2 receptor with moderate affinity (kD=56 nM±5).

Screening of this library resulted in the identification of a lead clamped SARS-CoV-2 S-protein (subsequently named SARS-CoV-2 Sclamp). The lead candidate incorporated the native signal peptide (site ‘a’ FIG. 2), replacement of the S1/S2 furin cleavage site amino acids 680-690 with a GSG flexible linker (site ‘b’ FIG. 2), the native sequence at S2′ proteolytic cleavage site and fusion peptide (site ‘c’ FIG. 2), the native sequence at amino acid 986/987 (site ‘d’ FIG. 2) and insertion of the molecular clamp coding sequence at amino acid 1,204 (site ‘e’ FIG. 2). Of note, the previously reported ‘double proline’ mutation (Wrapp, D. et al., 2020. supra; Kirchdoerfer, R. N. et al., 2018. supra) was not found to provide any advantage when included in addition to the molecular clamp and therefore was not included in the SARS-CoV-2 Sclamp construct.

The primary sequence of SARS-CoV-2 Sclamp is shown below:

[SEQ ID NO: 71] [symbol]
CVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNGSGSIIA
YTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTP
PIKDFGGFNFSQILPDPSKPSKRSFIEDLLFNKVTLADAGFIKQYGDCL
GDIAARDLICAQKFNGLTVLPPLLTDEMIAQYTSALLAGTITSGWTFGA
GAALQIPFAMQMAYRFNGIGVTQNVLYENQKLIANQFNSAIGKIQDSLS
STASALGKLQDVVNQNAQALNTLVKQLSSNFGAISSVLNDILSRLDKVE
AEVQIDRLITGRLQSLQTYVTQQLIRAAEIRASANLAATKMSECVLGQS
KRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNN
TVYDPLQPELDSFKEELDKYFKNHTSPDVDLGDISGINASVVNIQKEID
RLNEVAKNLNESLIDLQELGSGIVQQQNNLLRAIEAQQHLLQLTVWGIK
QLQARILAGGSGGHTTWMEWDREINNYTSLIHSLIEESQNQQEKNEQEL
LE,

wherein:
   Italicized bold text corresponds to signal peptide removed during expression and absent from final product;
   Underlined text corresponds to N-linked glycosylation sites;
   Non-italicized bold text corresponds to a flexible linker GSG inserted in place of native furin-like cleavage site; and
   Italicized non-bolded text corresponds to an HIV GP160-based SSM.

This lead candidate, SARS-CoV-2 Sclamp, showed an approximate expression level post-purification of 15 mg/L of transient ExpiCHO culture at Day 6. Analysis of the purified product by SDS-PAGE revealed the presence of a single molecular weight (MW) product at approximately 200 kDa, consistent with the expected size of the SARS-CoV-2 Sclamp. SEC revealed the presence of a major peak (˜60% of total) at an approximate MW of 600 kDa consistent with a soluble trimeric product. A minor peak was present at the void volume intermittent region indicative of substantial High Molecular Weight (HMW) aggregated product.

Transient gene expression in Chinese Hamster Ovary (CHO) cells (ExpiCHO-S, ThermoFisher) was initially assessed to allow rapid screening of antigen design panels. Transition to CHO stable cell lines (GS Xceed CHO-S system, Lonza) followed lead candidate selection. Using transient ExpiCHO expression the present inventors obtained expression levels of −20 mg/L. Transitioning to GS Xceed CHO-S stable pools, and using a fed batch bioprocess, resulted in expression in shaker flasks of 100-150 mg/L and in bioreactors up to 500 mg/L (FIG. 1). This level of mammalian cell based protein expression is an order of magnitude higher than that previously reported for an “optimized” spike protein construct (Hsieh, C. L. et al., 2020. bioRxiv, doi:10.1101/2020.05.30.125484) and creates the potential to generate many millions of doses per bioreactor run using industry standard 2,000 L single use bioprocess facilities.

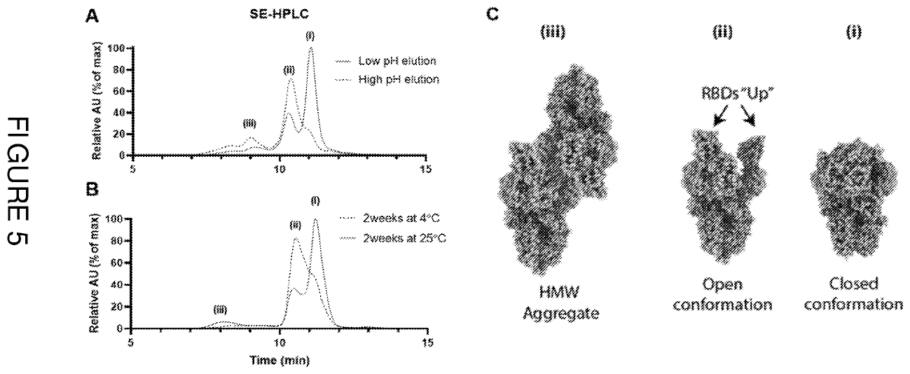

A 5 Å resolution structure was produced for SARS-CoV-2 Sclamp in the closed conformation by Cryo-TEM (FIG. 1). Further TEM revealed that the antigen can also adopt the open pre-fusion conformation (Wrapp, D. et al., 2020. Science 367:1260-1263, doi:10.1126/science.abb2507). Using SE-HPLC the antigen was demonstrated to exists in an equilibrium between these two conformations. This equilibrium could be transitioned between one or other conformation by exposure to varying pH or temperature (FIG. 5).

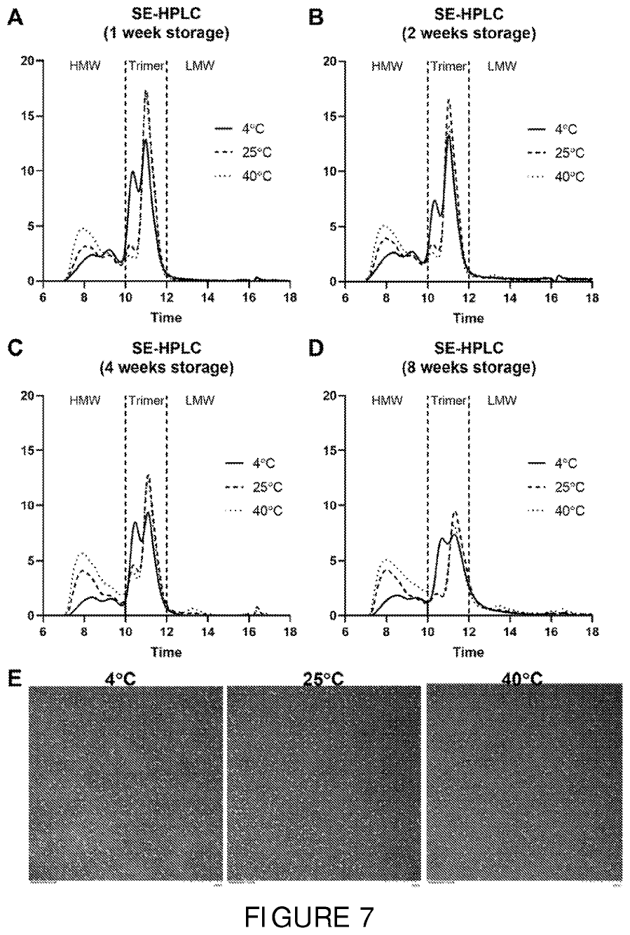

A panel of S specific mAbs including those originally identified for SARS-CoV (CR3022; ter Meulen, J. et al., 2006. PLoS Med 3:e237, doi:10.1371/journal.pmed.0030237) and newly identified mAbs to SARS-CoV-2 (S309; Pinto, D. et al., 2020. Nature 583:290-295, doi:10.1038/s41586-020-2349-y, and CB6; Hurlburt, N. K. et al., 2020. bioRxiv, doi:10.1101/2020.06.12.148692), were recombinantly engineered and shown to bind SARS-CoV-2 Sclamp with high affinity (CR3022 kDa=0.13 nM; S309 kDa=0.08 nM; CB6 kDa=0.15 nM). Recombinant hACE2 was found to bind to SARS-CoV-2 Sclamp with a similar affinity to that reported by others (Wrapp, D. et al., 2020. supra; Lan, J. et al., 2020. Nature 581:215-220, doi:10.1038/s41586-020-2180-5). Together, these findings further support the suggestion that SARS-CoV-2 Sclamp adopts a native conformation. The present inventors also demonstrate that antibody and receptor binding is retained by SARS-CoV-2 Sclamp even after prolonged exposure to heat stress, indicating that in combination with future formulation optimization, a long-shelf life may be achievable without the need for low temperature storage (FIGS. 1 and 7).

### Example 2

### Humoral Immune Response

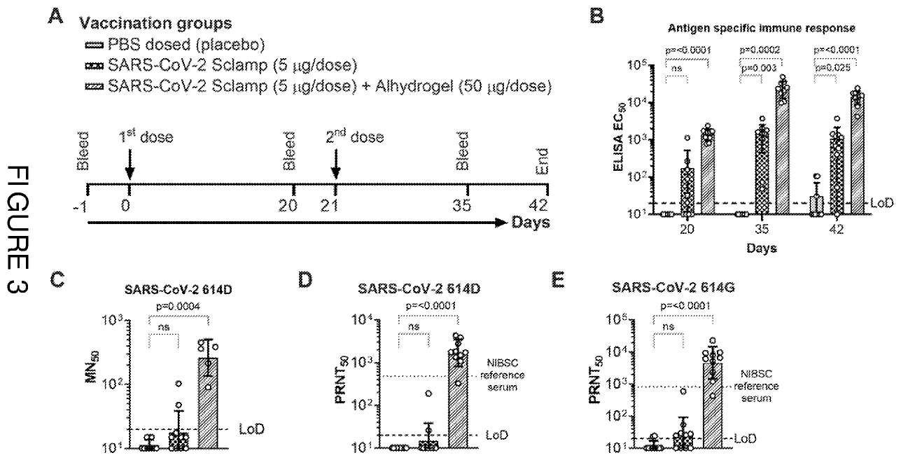

Highly purified or recombinant antigens may be co-administered with an adjuvant to enhance immunogenicity and to reduce the total amount of antigen required to provide protective immunity (Vogel, F. R., 2000. Clin Infect Dis 30 Suppl 3:S266-270, doi:10.1086/313883. Alhydrogel (Croda) was included as an adjuvant. BALB/c mice received intramuscular injections of PBS (placebo) or two doses of SARS-CoV-2 Sclamp (Ag) with or without Alhydrogel (FIG. 3A). Vaccinated mice developed a robust antigen-specific IgG response after a single dose with adjuvant and this was boosted following the second dose (FIG. 3B).

All mice receiving two doses of SARS-CoV-2 Sclamp with Alhydrogel produced a neutralizing antibody response as assessed in a microneutralization (MN) assay (FIG. 3C). Virus neutralization was observed equally for both D614 and G614 SARS-CoV2 variants using serum and bronchoalveolar lavage samples (FIGS. 3D and 3E).

### Example 3

### Cellular Immune Response

The present inventors evaluated the SARS-CoV-2 S-specific CD4⁺ and CD8⁺ T cell responses in vivo using a fluorescent target array (FTA) analysis and a complementary intracellular cytokine staining (ICS) analysis to determine the type 1 vs. type 2 immunity (Khanna, M. et al., 2019. Sci Rep 9(1):5661; Wijesundara, D. K. et al., 2014. PLoS One 29; 9(8):e105366).

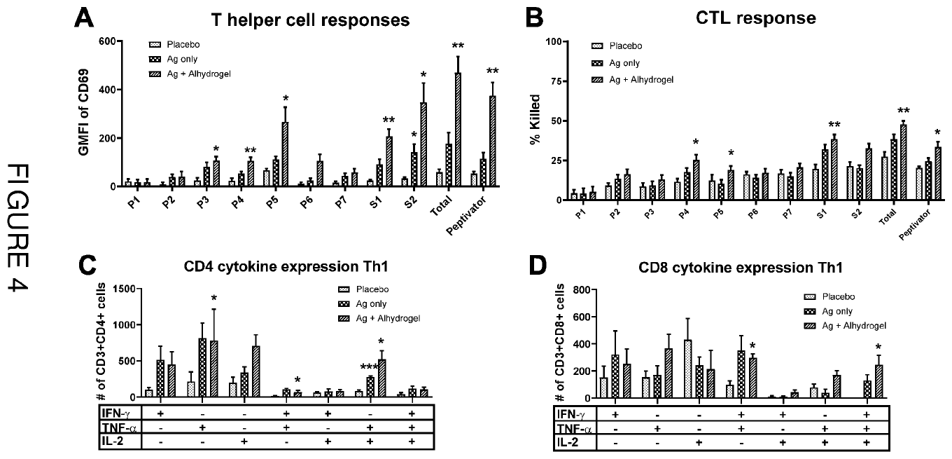

The FTA analysis involved challenging mice with fluorescent-bar coded cells pulsed with peptide pools that collectively span the SARS-CoV-2 S₁₋₁₂₂₆ sequence or a commercially available array of in silico mapped immunodominant peptides (Peptivator, Miteny Biotec) (FIG. 4). In the FTA, up-regulation of CD69 on S peptides-pulsed B220+ cells recovered from challenged mice is dependent on, and indicative of, antigen-specific Th cell responses with the presence of cytotoxic T lymphocyte (CTL) responses resulting in killing of the cognate peptides-pulsed targets in vivo (Mekonnen, Z. A. et al., 2019. J Virol 93: doi:10.1128/JVI.00202-19; Grubor-Bauk, B. et al., 2019. Sci Adv 5(12):eaax2388).

These findings showed that the Alhydrogel adjuvanted vaccination regimen was more effective in eliciting S-specific Th cell and CTL responses compared to Ag only and placebo control groups (FIG. 4).

### Discussion of Examples 1-3

The Sclamp, particularly in combination with Alhydrogel, was found to elicit a robust humoral immune response that was capable of efficiently neutralizing both the original strain and the D614G variant which has since become the dominant circulating strain. The present inventors confirmed that the vaccine elicited neutralizing antibodies against a broad array of epitopes, including those both inside and outside the RBD. This finding provides confidence that the breadth of vaccine efficacy will extend to drifted strains, and minimize the potential impact of the emergence of escape mutants (Baum, A. et al., 2020. Science, doi:10.1126/science.abd0831).

Overall, this work demonstrates the ability of SARS-CoV-2 Sclamp formulated with adjuvant to provide protection against SARS-CoV-2 infection and supports progression into human clinical trials and continued development.

### Example 4

### Clamp Silencing for Preventing Off-Target Immunogenicity

The present inventors sought to decrease elicitation of an immune response to molecular clamp domain, to more effectively focus the stimulation of an immune response against the virus. To achieve this, a version of the SARS-CoV-2 spike protein was produced comprising 5 N-linked glycosylation sites that are surface exposed and situated along the length of the clamp domain, as illustrated in the following sequence:

[SEQ ID NO: 75] [symbol]
QCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNGSGSIIA
YTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTP
PIKDFGGFNFSQILPDPSKPSKRSFIEDLLFNKVTLADAGFIKQYGDCL
GDIAARDLICAQKFNGLTVLPPLLTDEMIAQYTSALLAGTITSGWTFGA
GAALQIPFAMQMAYRFNGIGVTQNVLYENQKLIANQFNSAIGKIQDSLS
STASALGKLQDVVNQNAQALNTLVKQLSSNFGAISSVLNDILSRLDKVE
AEVQIDRLITGRLQSLQTYVTQQLIRAAEIRASANLAATKMSECVLGQS
KRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNN
TVYDPLQPELDSFKEELDKYFKNHTSPDVDLGDISGINASVVNIQKEID
RLNEVAKNLNESLIDLQELGSGIVQQQNNLLRAIEAQQHLLQLTVWGIK
QLQARILAGGSGGNHTTWMNWSREINNYTSLIHNLTEESQNQTEKNEQE
LLE,

wherein:
   Italicized bold text corresponds to signal peptide removed during expression and absent from final product;
   Underlined text corresponds to N-linked glycosylation sites;
   Non-italicized bold text corresponds to a flexible linker GSG inserted in place of native furin-like cleavage site; and
   Italicized non-bolded text corresponds to an immunosilenced HIV GP160-based SSM comprising 5× N-linked glycosylation sites.

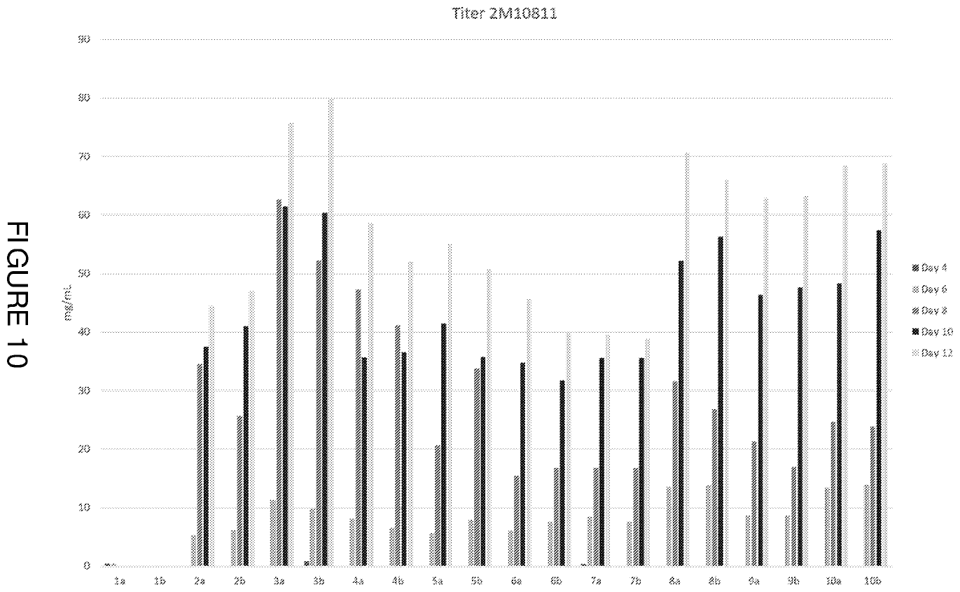

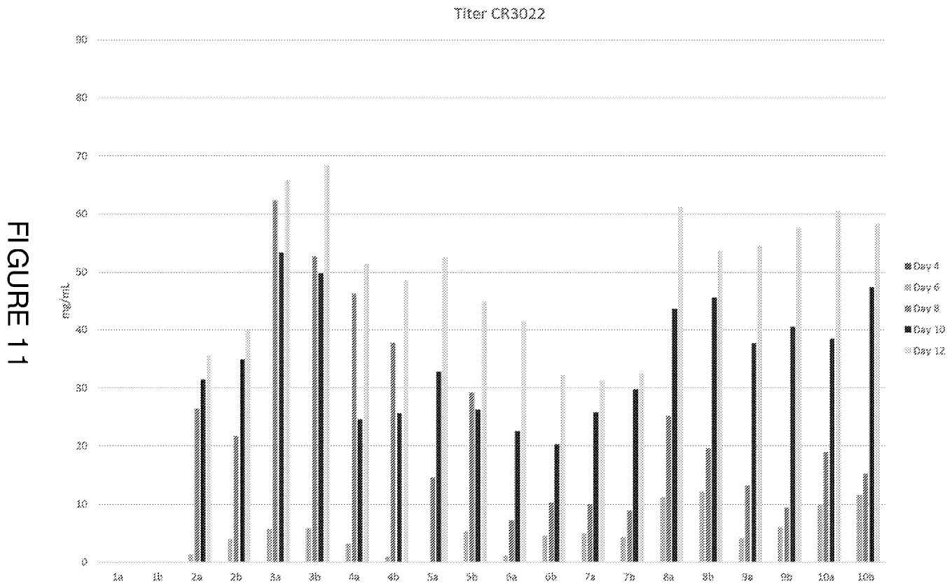

Stable cell lines were selected using the Lonza CHO-S GS-Xceed platform. The expression level of SARS-CoV-2 S-silenced-clamp was assessed via BIAcore by affinity with 2 different spike protein specific antibodies, CR3022 and 2M10B11 (FIGS. 10 and 11). The expression level for the highest expressing stable cell lines were estimated to be between 50 and 80 mg/L, which is consistent with the results obtained previously with the non-silenced clamp antigen.

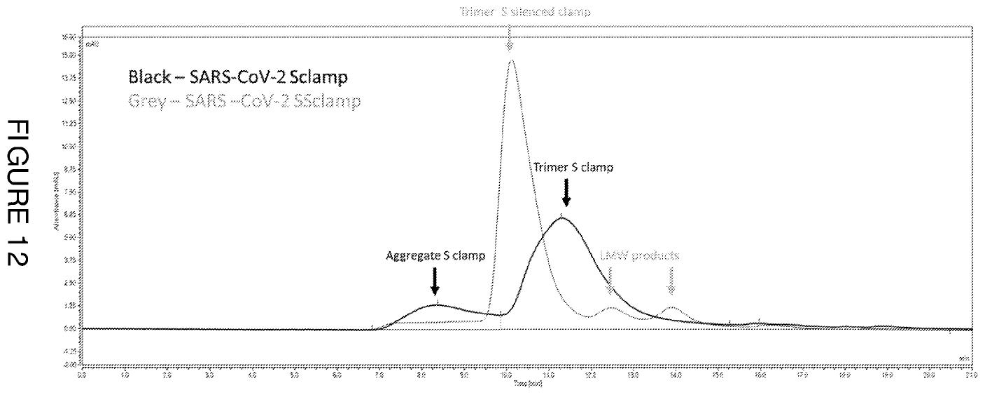

The SARS-CoV-2 S-silenced-clamp (SSclamp) was then purified from the cell supernatant by immunoaffinity chromatography using the monoclonal antibody 2M10B11 and eluted with pH 11.5. The purified antigen was then analyzed by SE-HPLC on a Waters X-Bridge 450 Å 300 mm and Guard column (FIG. 12). SE-HPLC analysis shows that the SARS-CoV-2 SSclamp is slightly larger than Sclamp as expected due to the incorporation of additional N-linked glycans to the clamp domain. Notably SSclamp showed no presence of aggregation which has been seen when the clamped version is produced.

Taken together these results confirm that the incorporation of silenced clamp in place of clamp does not negatively impact expression level or binding to spike specific antibodies. Importantly the silenced clamp appears to significantly decrease protein aggregation, which would be beneficial for production of a stable antigen for vaccinations.

### Example 5

### Design, In Vitro Analysis and Manufacturability of Additional SARS-Cov-2 S-Protein Vaccine Candidates Using Viral Derived Clamp Proteins

The lead candidate identified in Example 1, comprising the native signal peptide (site ‘a’ FIG. 2), replacement of the S1/S2 furin cleavage site amino acids 680-690 with a GSG flexible linker (site ‘b’ FIG. 2), the native sequence at S2′ proteolytic cleavage site and fusion peptide (site ‘c’ FIG. 2), the native sequence at amino acid 986/987 (site ‘d’ FIG. 2) is amplified by PCR and is cloned into plasmids upstream of alternative SSMs derived from RSV F, hMPV F, PIV F, MEV F, HEV F, Inf A HA, Inf B HA, EBOV GP, MARV GP, MERS S and SARS S proteins.

The primary sequence of SARS-CoV-2 Sclamp-RSV is shown below:

[SEQ ID NO: 87] [symbol]
QCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNGSGSIIA
YTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTP
PIKDFGGFNFSQILPDPSKPSKRSFIEDLLFNKVTLADAGFIKQYGDCL
GDIAARDLICAQKFNGLTVLPPLLTDEMIAQYTSALLAGTITSGWTFGA
GAALQIPFAMQMAYRFNGIGVTQNVLYENQKLIANQFNSAIGKIQDSLS
STASALGKLQDVVNQNAQALNTLVKQLSSNFGAISSVLNDILSRLDKVE
AEVQIDRLITGRLQSLQTYVTQQLIRAAEIRASANLAATKMSECVLGQS
KRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNN
TVYDPLQPELDSFKEELDKYFKNHTSPDVDLGDISGINASVVNIQKEID
RLNEVAKNLNESLIDLQELGSGVNKIKSALLSTNKAVVSLSNGVSVLTS
KVLDLKGGSGGFDASISQVNEKINQSLAFIRKSDELLHNVNAGKSTTN

The primary sequence of SARS-CoV-2 Sclamp-hMPV F is shown below:

[SEQ ID NO: 88] [symbol]
QCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNGSGSIIA
YTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTP
PIKDFGGFNFSQILPDPSKPSKRSFIEDLLFNKVTLADAGFIKQYGDCL
GDIAARDLICAQKFNGLTVLPPLLTDEMIAQYTSALLAGTITSGWTFGA
GAALQIPFAMQMAYRFNGIGVTQNVLYENQKLIANQFNSAIGKIQDSLS
STASALGKLQDVVNQNAQALNTLVKQLSSNFGAISSVLNDILSRLDKVE
AEVQIDRLITGRLQSLQTYVTQQLIRAAEIRASANLAATKMSECVLGQS
KRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNN
TVYDPLQPELDSFKEELDKYFKNHTSPDVDLGDISGINASVVNIQKEID
RLNEVAKNLNESLIDLQELGSGKTIRLESEVTAIKNALKKTNEAVSTLG
NGVRVLATAVRELKDFVGGSGGFNVALDQVFESIENSQALVDQSNRILS
SAEKGNTG

The primary sequence of SARS-CoV-2 Sclamp-PIV is shown below:

[SEQ ID NO: 89] [symbol]
QCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNGSGSIIA
YTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTP
PIKDFGGFNFSQILPDPSKPSKRSFIEDLLFNKVTLADAGFIKQYGDCL
GDIAARDLICAQKFNGLTVLPPLLTDEMIAQYTSALLAGTITSGWTFGA
GAALQIPFAMQMAYRFNGIGVTQNVLYENQKLIANQFNSAIGKIQDSLS
STASALGKLQDVVNQNAQALNTLVKQLSSNFGAISSVLNDILSRLDKVE
AEVQIDRLITGRLQSLQTYVTQQLIRAAEIRASANLAATKMSECVLGQS
KRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNN
TVYDPLQPELDSFKEELDKYFKNHTSPDVDLGDISGINASVVNIQKEID
RLNEVAKNLNESLIDLQELGSGEAKQARSDIEKLKEAIRDTNKAVQSVQ
SSIGNLGGSGGIELNKAKSDLEESKEWIRRSNQKLDSIGNWHQSSTT

The primary sequence of SARS-CoV-2 Sclamp-MEV F is shown below:

[SEQ ID NO: 90] [symbol]
QCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNGSGSIIA
YTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTP
PIKDFGGFNFSQILPDPSKPSKRSFIEDLLFNKVTLADAGFIKQYGDCL
GDIAARDLICAQKFNGLTVLPPLLTDEMIAQYTSALLAGTITSGWTFGA
GAALQIPFAMQMAYRFNGIGVTQNVLYENQKLIANQFNSAIGKIQDSLS
STASALGKLQDVVNQNAQALNTLVKQLSSNFGAISSVLNDILSRLDKVE
AEVQIDRLITGRLQSLQTYVTQQLIRAAEIRASANLAATKMSECVLGQS
KRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNN
TVYDPLQPELDSFKEELDKYFKNHTSPDVDLGDISGINASVVNIQKEID
RLNEVAKNLNESLIDLQELGSGHQSMLNSQAIDNLRASLETTNQAIEAI
RQAGQEMGGSGGLERLDVGTNLGNAIAKLEDAKELLESSDQILRSMKGL
SST

The primary sequence of SARS-CoV-2 Sclamp-HEV F is shown below:

[SEQ ID NO: 91] [symbol]
QCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNGSGSIIA
YTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTP
PIKDFGGFNFSQILPDPSKPSKRSFIEDLLFNKVTLADAGFIKQYGDCL
GDIAARDLICAQKFNGLTVLPPLLTDEMIAQYTSALLAGTITSGWTFGA
GAALQIPFAMQMAYRFNGIGVTQNVLYENQKLIANQFNSAIGKIQDSLS
STASALGKLQDVVNQNAQALNTLVKQLSSNFGAISSVLNDILSRLDKVE
AEVQIDRLITGRLQSLQTYVTQQLIRAAEIRASANLAATKMSECVLGQS
KRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNN
TVYDPLQPELDSFKEELDKYFKNHTSPDVDLGDISGINASVVNIQKEID
RLNEVAKNLNESLIDLQELGSGEAMKNADNINKLKSSIESTNEAVVKLQ
ETAEKTVGGSGGISSQISSMNQSLQQSKDYIKEAQKILDTVNPS

The primary sequence of SARS-CoV-2 Sclamp-INF A HA is shown below:

[SEQ ID NO: 92] [symbol]
QCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNGSGSIIA
YTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTP
PIKDFGGFNFSQILPDPSKPSKRSFIEDLLFNKVTLADAGFIKQYGDCL
GDIAARDLICAQKFNGLTVLPPLLTDEMIAQYTSALLAGTITSGWTFGA
GAALQIPFAMQMAYRFNGIGVTQNVLYENQKLIANQFNSAIGKIQDSLS
STASALGKLQDVVNQNAQALNTLVKQLSSNFGAISSVLNDILSRLDKVE
AEVQIDRLITGRLQSLQTYVTQQLIRAAEIRASANLAATKMSECVLGQS
KRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNN
TVYDPLQPELDSFKEELDKYFKNHTSPDVDLGDISGINASVVNIQKEID
RLNEVAKNLNESLIDLQELGSGENGWEGMVDGWYGFRHQNSEGTGQAAD
LKSTQAAIGGSGGRIQDLEKYVEDTKIDLWSYNAELLVALENQHTIDLT
DSEMSKLFERTRR

The primary sequence of SARS-CoV-2 Sclamp-INF B is shown below:

[SEQ ID NO: 93] [symbol]
QCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNGSGSIIA
YTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTP
PIKDFGGFNFSQILPDPSKPSKRSFIEDLLFNKVTLADAGFIKQYGDCL
GDIAARDLICAQKFNGLTVLPPLLTDEMIAQYTSALLAGTITSGWTFGA
GAALQIPFAMQMAYRFNGIGVTQNVLYENQKLIANQFNSAIGKIQDSLS
STASALGKLQDVVNQNAQALNTLVKQLSSNFGAISSVLNDILSRLDKVE
AEVQIDRLITGRLQSLQTYVTQQLIRAAEIRASANLAATKMSECVLGQS
KRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNN
TVYDPLQPELDSFKEELDKYFKNHTSPDVDLGDISGINASVVNIQKEID
RLNEVAKNLNESLIDLQELGSGHGYTSHGAHGVAVAADLKSTQEAINKI
TKNLNYLGGSGGDEILELDEKVDDLRADTISSQIELAVLLSNEGIINSE
DEHLLALERKLKKML

The primary sequence of SARS-CoV-2 Sclamp-EBOV GP is shown below:

[SEQ ID NO: 94] [symbol]
QCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNGSGSIIA
YTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTP
PIKDFGGFNFSQILPDPSKPSKRSFIEDLLFNKVTLADAGFIKQYGDCL
GDIAARDLICAQKFNGLTVLPPLLTDEMIAQYTSALLAGTITSGWTFGA
GAALQIPFAMQMAYRFNGIGVTQNVLYENQKLIANQFNSAIGKIQDSLS
STASALGKLQDVVNQNAQALNTLVKQLSSNFGAISSVLNDILSRLDKVE
AEVQIDRLITGRLQSLQTYVTQQLIRAAEIRASANLAATKMSECVLGQS
KRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNN
TVYDPLQPELDSFKEELDKYFKNHTSPDVDLGDISGINASVVNIQKEID
RLNEVAKNLNESLIDLQELGSGGLRQLANETTQALQLFLRATTELRTFS
ILNRKAIDFLGGSGGTCHILGPDCCIEPHDWTKNITDKIDQIIHDFVDK
TL

The primary sequence of SARS-CoV-2 Sclamp-MARV GP is shown below:

[SEQ ID NO: 95] [symbol]
QCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNGSGSIIA
YTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTP
PIKDFGGFNFSQILPDPSKPSKRSFIEDLLFNKVTLADAGFIKQYGDCL
GDIAARDLICAQKFNGLTVLPPLLTDEMIAQYTSALLAGTITSGWTFGA
GAALQIPFAMQMAYRFNGIGVTQNVLYENQKLIANQFNSAIGKIQDSLS
STASALGKLQDVVNQNAQALNTLVKQLSSNFGAISSVLNDILSRLDKVE
AEVQIDRLITGRLQSLQTYVTQQLIRAAEIRASANLAATKMSECVLGQS
KRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNN
TVYDPLQPELDSFKEELDKYFKNHTSPDVDLGDISGINASVVNIQKEID
RLNEVAKNLNESLIDLQELGSGTFSLINRHAIDFLLTRWGGSGGIGIED
LSKNISEQIDQIK

The primary sequence of SARS-CoV-2 Sclamp-MERS S is shown below:

[SEQ ID NO: 96] [symbol]
QCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNGSGSIIA
YTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTP
PIKDFGGFNFSQILPDPSKPSKRSFIEDLLFNKVTLADAGFIKQYGDCL
GDIAARDLICAQKFNGLTVLPPLLTDEMIAQYTSALLAGTITSGWTFGA
GAALQIPFAMQMAYRFNGIGVTQNVLYENQKLIANQFNSAIGKIQDSLS
STASALGKLQDVVNQNAQALNTLVKQLSSNFGAISSVLNDILSRLDKVE
AEVQIDRLITGRLQSLQTYVTQQLIRAAEIRASANLAATKMSECVLGQS
KRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNN
TVYDPLQPELDSFKEELDKYFKNHTSPDVDLGDISGINASVVNIQKEID
RLNEVAKNLNESLIDLQELGSGGITQQVLSENQKLIANKFNQALGAMQT
GFTTTNEAFRKVQDAVNNNAQALSKLASELSNTFGAISASIGDIIQRLD
VLEQDAQIDRLINGRLTTLNAFVAQQLVRSESAALSAQLAKDKVNEGGS
GGNFGSLTQINTTLLDLTYEMLSLQQVVKALNESYIDLKELGNYTY

The primary sequence of SARS-CoV-2 Sclamp-SARS is shown below:

[SEQ ID NO: 97] [symbol]
QCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNGSGSIIA
YTMSLGAENSVAYSNNSIAIPTNFTISVTTEILPVSMTKTSVDCTMYIC
GDSTECSNLLLQYGSFCTQLNRALTGIAVEQDKNTQEVFAQVKQIYKTP
PIKDFGGFNFSQILPDPSKPSKRSFIEDLLFNKVTLADAGFIKQYGDCL
GDIAARDLICAQKFNGLTVLPPLLTDEMIAQYTSALLAGTITSGWTFGA
GAALQIPFAMQMAYRFNGIGVTQNVLYENQKLIANQFNSAIGKIQDSLS
STASALGKLQDVVNQNAQALNTLVKQLSSNFGAISSVLNDILSRLDKVE
AEVQIDRLITGRLQSLQTYVTQQLIRAAEIRASANLAATKMSECVLGQS
KRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNN
TVYDPLQPELDSFKEELDKYFKNHTSPDVDLGDISGINASVVNIQKEID
RLNEVAKNLNESLIDLQELGSGGVTQNVLYENQKQIANQFNKAISQIQE
SLTTTSTALGKLQDVVNQNAQALNTLVKQLSSNFGAISSVLNDILSRLD
KVEAEVQIDRLITGRLQSLQTYVTQQLIRAAEIRASANLAATKMSEGGS
GGDVDLGDISGINASVVNIQKEIDRLNEVAKNLNESLIDLQELGK

wherein:
   Italicized bold text corresponds to signal peptide removed during expression and absent from final product;
   Underlined text corresponds to N-linked glycosylation sites;
   Non-italicized bold text corresponds to a flexible linker GSG inserted in place of native furin-like cleavage site; and
   Italicized non-bolded text corresponds to the viral-derived SSM.

Each SARS-CoV-2 sClamp protein stabilised with a viral-derived SSM is cloned into a plasmid and transfected for transient expression into ExpiCHO cells from the ExpiCHO-S expression system (ThermoFisher Scientific). CHO cells are cultured in ExpiCHO-S Expression Medium (Gibco™) and transfection is conducted following the manufacturer's protocols for 5 or 7 days prior to harvest of the culture supernatant and protein purification. The proteins show an approximate yield level post-purification of 1-50 mg/L of transient ExpiCHO culture at Day 6. Analysis of the purified products by SDS-PAGE reveals the presence of a single molecular weight (MW) product, consistent with the expected size of the SARS-CoV-2 sClamp-viral SSM domains (Table 8).

Size exclusion chromatography reveals a major peak consistent with a soluble trimeric product (Table 8).

TABLE 8
Molecular weights of monomeric and trimeric
forms of SARS-CoV-2-sClamp-viral SSMs

| | Source | Monomer | Trimer |
|---|---|---|---|
| Protein | of SSM | (kDa) | (kDa) |
| SARS-CoV-2 Sclamp-RSV F | RSV F | 140 | 421 |
| SARS-CoV-2 Sclamp-hMPV F | hMPV F | 141 | 424 |
| SARS-CoV-2 Sclamp-PIV F | PIV F | 141 | 422 |
| SARS-CoV-2 Sclamp-MEV F | MEV F | 141 | 423 |
| SARS-CoV-2 Sclamp-HEV F | HEV F | 140 | 420 |
| SARS-CoV-2 Sclamp-Inf A HA | Inf A HA | 143 | 428 |
| SARS-CoV-2 Sclamp-Inf B HA | Inf B HA | 142 | 427 |
| SARS-CoV-2 Sclamp-EBOV GP | EBOV GP | 141 | 424 |
| SARS-CoV-2 Sclamp-MARV GP | MARV GP | 137 | 411 |
| SARS-CoV-2 Sclamp-MERS S | MERS S | 151 | 453 |
| SARS-CoV-2 Sclamp-SARS S | SARS S | 151 | 453 |

Antibody Binding

A panel of S specific mAbs including those originally identified for SARS-CoV (CR3022; ter Meulen, J. et al., 2006. PLoS Med 3:e237, doi:10.1371/journal.pmed.0030237) and newly identified mAbs to SARS-CoV-2 (S309; Pinto, D. et al., 2020. Nature 583:290-295, doi:10.1038/s41586-020-2349-y, and CB6; Hurlburt, N. K. et al., 2020. bioRxiv, doi:10.1101/2020.06.12.148692), were recombinantly engineered and are able to bind SARS-CoV-2-Sclamp viral SSMs with high affinity. Recombinant hACE2 is able to bind to SARS-CoV-2 Sclamp viral SSMs. Binding of these antibodies and recombinant hACE2 indicates that the SARS-CoV-2 spike protein adopts a native conformation.

Mouse Vaccinations, Humoral Immune Response and Viral Neutralisation

Groups of BALB/c mice receive two intramuscular injections of PBS (placebo) or two doses of SARS-CoV-2-Sclamp viral SSMs with or without Alhydrogel adjuvant.

Vaccinated mice develop a robust antigen-specific IgG response after a single dose with adjuvant that is boosted following a second dose.

Mice receiving two doses of SARS-CoV-2Sclamp viral SSMs with Alhydrogel produce a neutralizing antibody response in microneutralization (MN) assay.

Discussion

The SARS-CoV-2SClamp viral SSMs, particularly in combination with Alhydrogel, are found to elicit robust immune responses that are capable of efficiently neutralizing SARS-CoV-2 viruses. The SARS-CoV-2SClamp viral SSMs formulated with adjuvant can provide protection against SARS-CoV-2 infection.

### Example 6

### Design, In Vitro Analysis and Manufacturability of SARS-Cov-2 S Protein-Foldon Vaccine Candidate

The lead candidate identified in Example 1, comprising the native signal peptide (site ‘a’ FIG. 2), replacement of the S1/S2 furin cleavage site amino acids 680-690 with a GSG flexible linker (site ‘b’ FIG. 2), the native sequence at S2′ proteolytic cleavage site and fusion peptide (site ‘c’ FIG. 2), the native sequence at amino acid 986/987 (site ‘d’ FIG. 2) was amplified by PCR and was cloned into plasmids upstream of a foldon SSM (Meier et al. (2004), JMB, 344(4):1051-1069, doi:10.1016/j.jmb.2004.09.079).

The primary sequence of the SARS-CoV-2-foldon is shown below

[SEQ ID NO: 98] [symbol]
SQCVNLTTRTQLPPAYTNSFTRGVYYPDKVFRSSVLHSTQDLFLPFFSNVTWFHAIHVSGTN
GTKRFDNPVLPFNDGVYFASTEKSNIIRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGYFKIYSKHTPINLVRDLPQGFSALEP
LVDLPIGINITRFQTLLALHRSYLTPGDSSSGWTAGAAAYYVGYLQPRTFLLKYNENGTITDAVDCALDPLSETKC
TLKSFTVEKGIYQTSNFRVQPTESIVRFPNITNLCPFGEVFNATRFASVYAWNRKRISNCVADYSVLYNSASFSTF
KCYGVSPTKLNDLCFTNVYADSFVIRGDEVRQIAPGQTGKIADYNYKLPDDFTGCVIAWNSNNLDSKVGGNYNY
LYRLFRKSNLKPFERDISTEIYQAGSTPCNGVEGFNCYFPLQSYGFQPTNGVGYQPYRVVVLSFELLHAPATVCGP
KKSTNLVKNKCVNFNFNGLTGTGVLTESNKKFLPFQQFGRDIADTTDAVRDPQTLEILDITPCSFGGVSVITPGTN
TSNQVAVLYQDVNCTEVPVAIHADQLTPTWRVYSTGSNVFQTRAGCLIGAEHVNNSYECDIPIGAGICASYQTQ
TNGSGSIIAYTMSLGAENSVAYSNNSIAIPTNFTISVTTEILPVSMTKTSVDCTMYICGDSTECSNLLLQYGSFCT
QLNRALTGIAVEQDKNTQEVFAQVKQIYKTPPIKDFGGFNFSQILPDPSKPSKRSFIEDLLFNKVTLADAGFIKQY
GDCLGDIAARDLICAQKFNGLTVLPPLLTDEMIAQYTSALLAGTITSGWTFGAGAALQIPFAMQMAYRFNGIGVT
QNVLYENQKLIANQFNSAIGKIQDSLSSTASALGKLQDVVNQNAQALNTLVKQLSSNFGAISSVLNDILSRLDKV
EAEVQIDRLITGRLQSLQTYVTQQLIRAAEIRASANLAATKMSECVLGQSKRVDFCGKGYHLMSFPQSAPHGVVF
LHVTYVPAQEKNFTTAPAICHDGKAHFPREGVFVSNGTHWFVTQRNFYEPQIITTDNTFVSGNCDVVIGIVNNTV

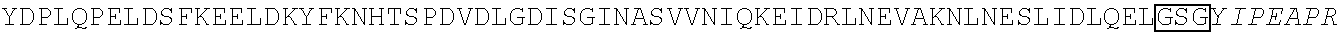

DGQAYVRKDGEWVLLSTFGSGEPEA

wherein:
   Italicized bold text corresponds to signal peptide removed during expression and absent from final product;
   Non-italicized bold text corresponds to a flexible linker GSG inserted in place of native furin-like cleavage site;
   Boxed text corresponds to a GSG linker. Note the first G is native to SARS-CoV-2 spike protein; and
   Underlined text is a c-tag purification sequence

SARS-CoV-2-foldon was cloned into a plasmid and transfected for transient expression into ExpiCHO cells from the ExpiCHO-S expression system (ThermoFisher Scientific). CHO cells were cultured in ExpiCHO-S Expression Medium (Gibco™) and transfection was conducted following the manufacturer's protocols for 5 or 7 days prior to harvest of the culture supernatant and protein purification.

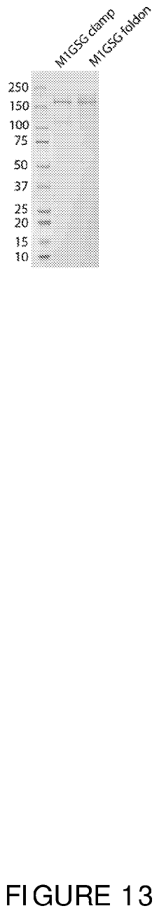

Analysis of culture cell supernatants indicated that the SARS-CoV-2-foldon was expressed at high levels, with the size of the expressed protein consistent with the expected size of the SARS-CoV-2-foldon (135 kDa, FIG. 13).

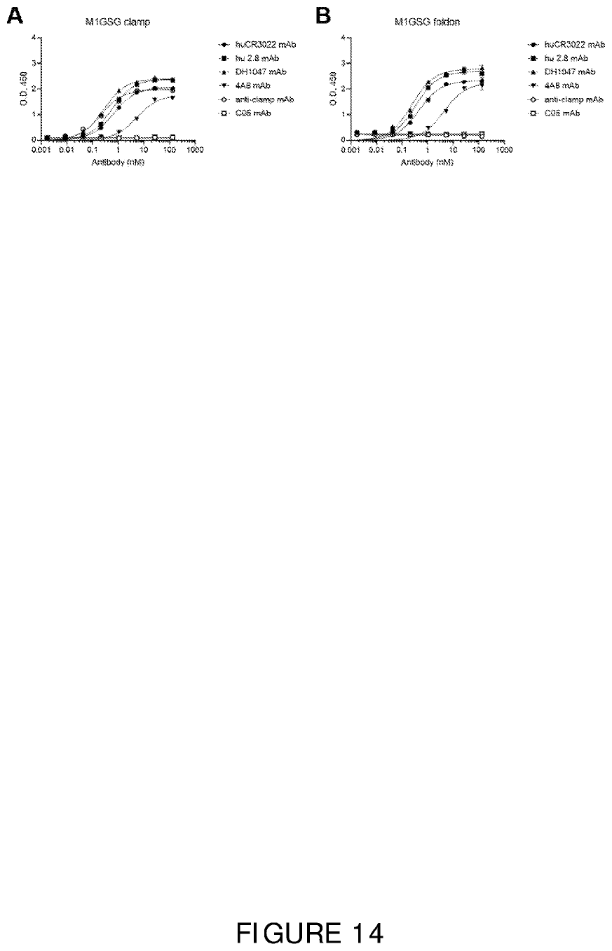

SARS-CoV-2-foldon was found to react with conformationally specific antibodies to the SARS-CoV-2 spike protein (FIG. 14).

### Example 7

### Materials and Methods

### Constructs and Plasmids

To express the prefusion S ectodomain, codon-optimized SARS-CoV2 S (GenBank accession number: MN908947) gene with variations including (i) substitution at the furin cleavage site (ii) substitution at signal peptide (iii) truncation at C-terminal domain was generated with primers containing overlapping sequence by PCR mutagenesis using Phusion polymerase (New England Biolabs). These amplicons were introduced upstream of HIV1281 (PDB ID 3P30) trimerization motif. To express the SARS-CoV-2 RBD-SD1 (aa residues 319-526), was cloned upstream of a human Fc tag. A human ACE2 ectodomain (residues 20-602) was cloned into human monomeric Fc tag. Plasmid encoding variable domains of heavy and light chain of CR3022 (ter Meulen, J. et al., 2006, supra) S309 (Pinto, D. et al., 2020, supra), B38 (Pinto, D. et al., 2020, supra), H4 (Huo, J. et al., 2020. Nat Struct Mol Biol, doi:10.1038/s41594-020-0469-6), CB6 (Pinto, D. et al., 2020, supra), G4 (anti-MERS S)(Wang, L. et al., 2015. Nat Commun 6:7712, doi:10.1038/ncomms8712) and anti-clamp HIV1281 (Frey, G. et al., 2010. Nat Struct Mol Biol 17:1486-1491, doi:10.1038/nsmb.1950) were cloned into the mammalian expression vector, pNBF-Hv or pNBF-Lv in-frame with IgK signal peptide.

### Recombinant Protein Expression

The ExpiCHO-S expression system (ThermoFisher Scientific) was used for transient Spike protein and antibody expression. CHO cells were cultured in ExpiCHO-S Expression Medium (Gibco™) and transfection was conducted following the manufacturer's protocols for 5 or 7 days prior to harvest of the culture supernatant and protein purification. Stable cell lines were generated using the Lonza GS Xceed® System. CHOK1SV GS-KO® cells were transfected via electroporation with linearized GS expression vector expressing SARS-CoV-2 Sclamp as per manufactures instructions (GS Xceed® manual, Version 06 2019). Approximately 24 hours later enriched pools were selected using 50 μM L-Methionine sulfoximine (MSX) over a period of 3-4 weeks. Stable pool shaker flask expression was assessed over 12 days via Lonza's abridged fed-batch shake flask screen (v8.10) and clone selection was performed using the Beacon Optofluidic platform (Berkeley Lights). Stable pools were loaded onto the OptoSelect™ 1750b Chip as single cells. Cells were cultured on chip for 3-5 days before pens were analyzed for secretion of SARS-CoV-2 Sclamp using fluorescently tagged anti-Clamp IgG. Selected pens were then exported into a 96-well plate and scaled-up into shaker flasks. Clones were further assessed via Lonza's abridged fed-batch shake flask screen (v8.10).

### SDS-PAGE and Western Blot

CHO supernatants from transfected cultures and purified proteins were screened for expression of Sclamp vaccine candidates via SDS-PAGE for protein homogeneity and Western blot. Supernatants and proteins separated via SDS-PAGE on a 4-15% Mini-PROTEAN TGX Precast Protein gel (Bio-Rad). For Western Blot analysis, proteins were transferred using the iBlot 2 Dry Blotting system (ThermoFisher) before blocking using 5% KPL Milk Diluent/Blocking solution concentrate (SeraCare) in PBS with 0.05% Tween 20 for 30 minutes on an orbital shaker. Blocking solution was removed and the membrane incubated with the anti-clamp MAb HIV1281 for 1 h at room temperature (RT) with shaking. The membrane was washed three times with PBS containing 0.05% Tween 20 for five minutes each before the addition of a 1:2500 dilution of an IRDye 800CW goat anti-human secondary antibody (LI-COR Biosciences) for 1 h with shaking. The membrane was washed as before and imaged using an Odyssey CLx infrared imager (LI-COR Biosciences).

### Recombinant Protein Purification

SARS-CoV-2 stabilized spike protein was purified using immunoaffinity chromatography on an ÄKTA pure protein purification system (Cytiva). This was achieved using an in-house made immunoaffinity chromatography column—the anti-clamp MAb HIV1281 coupled to 1 or 5 mL HiTrap-NHS activated HP Columns (Cytiva). CHO expression culture was centrifuged at 4000×g for 10 min at 4° C. and resultant supernatant filtered through a filter unit (0.22 μm pore size). Supernatant was added to anti-spike protein affinity column that was pre-equilibrated with high salt PBS (PBS with 400 mM NaCl, pH 7.4). Bound resin was washed with 15 column volumes (CV) of high salt PBS before elution with either high pH buffer (100 mM glycine, 137 mM NaCl, 5 mM EDTA pH 11.5) or low pH buffer (100 mM Sodium Acetate, 100 mM NaCl, pH 3.5). Antibodies were purified from cell supernatants using Protein A HP column (Cytiva). Eluted fractions were neutralized with a 1:1 v/v ratio of 1M Tris pH 6.8 before concentration and buffer exchange into PBS using Merck Amicon Ultra-4 or Ultra-15 centrifugal filter units. Protein concentration was determined using the NanoDrop One (ThermoFisher) or via the Pierce BCA protein assay kit (ThermoFisher).

### Size Exclusion Chromatography (SEC) and High Pressure Liquid Chromatography (SE-HPLC) of Stabilized Sclamp Protein

To assess oligomeric state of recombinant spike proteins, 30 μg of purified recombinant protein was loaded onto a Superose 6™ Increase 10/300 GL size-exclusion chromatography column (Cytiva) using a 300-500 μL loop. Proteins were eluted using a mobile phase of PBS, pH 7.4 at a flow rate of 0.3-0.5 mL/min. Higher resolution assessment of oligomeric state was conducted using an Agilent 1200 HPLC. Duplicate 95 μL samples were injected onto a Waters X-Bridge 300 mm column pre-calibrated with PBS mobile phase and using a flow rate of 0.8 ml/min.

### Negative Staining Electron Microscopy of Pre-Fusion Sclamp Protein

SARS-CoV-2 proteins were diluted at −10 μg/mL in PBS. Diluted proteins (4 mL) were adsorbed onto carbon-coated grids (ProSciTech) for 2 min and glow discharged for 5 sec in 25 mA. The grids were blotted and washed three time in water and stained twice with 1% Uranyl acetate with blotting in between. The grids were air dried and imaged using a Hitachi HT7700 microscope operated at 120 Kv.

### Thermal Stability Testing

Purified antigen was sterile-filtered and diluted in PBS to a final concentration of mg/mL. 250 μl aliquots were added to 1.5 mL sterile microcentrifuge tubes which were then stored at either 4° C., 25° C. or 40° C. for 1, 2, 4, or 8 weeks. At each designated time point, samples were removed from incubation and assessed by ELISA and SE-HPLC.

### Mouse Vaccinations and Immune Analysis

Five- to seven-week old female BALB/c mice were purchased from the Australian Resource Centre, Perth and housed in individually ventilated HEPA-filtered cages at the University of Queensland Biological Resources facility, The Australian Institute for Bioengineering and Nanotechnology. The mice were allowed to acclimatize for at least 5 days prior to vaccination via the intramuscular (IM) route using the hind leg muscle with 50 μL of PBS (placebo) or 5 μg/mouse of SARS-CoV-2 Sclamp with or without Alhydrogel (50 μg/mouse, InvivoGen) under anesthesia.

Blood was collected via the tail vein prior to each vaccination and using cardiac puncture at the study end point and serum was collected via centrifugation for 10 min at 10000×g of blood samples that were stored at 4° C. overnight. Serum was heat inactivated at 56° C. for 30 min and stored at −80° C. prior to analysis.

BAL fluid was collected following perfusion of the lungs via the trachea using 400 μl of PBS. The cells found in the BAL fluid were removed following pelleting of the cells from each sample at 300×g for 7 min at 4° C.

To assess the T cell response, red blood cell (RBC) depleted splenocytes from each mouse was isolated at the study end point and analyzed using ICS or the FTA as described below.

### ELISA

A capture ELISA was used to screen ExpiCHO-S supernatants and purified proteins for expression of Sclamp vaccine candidates. Nunc MaxiSorp™ ELISA plates were coated with 2 μg/mL of the anti-Clamp MAb HIV1281 in PBS overnight at 4° C. Plates were then blocked with 150 μL/well of 5% KPL Milk Diluent/Blocking solution concentrate (SeraCare) in PBS with 0.05% Tween 20 for 1 h at room temperature. Blocking buffer was removed and plates were incubated with serial dilutions of harvested ExpiCHO-S supernatant for one hour at 37° C. Plates were washed three times with water before incubation with 5 μg/mL of an in-house produced recombinant CR3022 MAb (ter Meulen, J. et al., 2006, supra) in a mouse IgG1 backbone for one hour at 37° C. Following another wash step, plates were incubated with a 1:2000 dilution of a horse radish peroxidase (HRP) conjugated goat anti-mouse secondary antibody (#A16072) for 1 h at 37° C. After a final wash, plates were developed for five minutes using TMB Single solution chromogen/substrate (#002023) before the reaction stopped by addition of 2N H₂SO₄. Absorbance at 450 nm was then read on a Spectramax 190 Microplate reader (Molecular Devices).

For ELISA analysis of mouse serum from placebo or Sclamp immunized mice, Nunc MaxiSorp™ ELISA plates were coated with 2 μg/mL of antigen and blocked as above. The blocked plates were incubated with serially diluted mouse serum at 37° C. for 1 hr. The plates were then washed, developed and read as described above. EC50 values were calculated by three parameter curve fitting using nonlinear regression in GraphPad Prism (version 8.3.1). The Limit of Detection (LoD) was defined as the reciprocal of the highest concentration of sera tested and any values falling below the LoD were reported as ½ LoD.

For the depletion ELISA analysis of mouse serum, 10 μg/mL of the depleting antigen was added to titrated mouse serum in a 96-well round bottom plate. Antigens used for depletion included an alternate clamp stabilized viral glycoprotein (i.e., influenza virus haemagglutinin (HA)clamp) and SARS-CoV-2 RBD. Sera and depletion antigen were incubated at 37° C. for 1 h prior to addition to the ELISA plate and analysis as described above. The ELISA plate setup included separate rows coated with the depletion antigen as a control for incomplete depletion of domain specific response.

### Microneutralisation (Mn) Assay

Neutralizing activity against live SARS-CoV-2 was assessed by a traditional MN assay as originally described for SARS-CoV (Subbarao, K. et al., 2004. J Virol 78:3572-3577, doi:10.1128/jvi.78.7.3572-3577). Briefly, Vero cells were seeded into flat-bottom 96 well tissue culture plates. The following day cells were washed with serum free MEM and fresh MEM containing 1 μg/mL TPCK trypsin. In a separate 96 well round bottom plate a 1:2 serial dilution of serum was performed across the plate starting at 1:10 with 125 μL of media per well, leaving last 2 columns (virus control and cell control). An equal volume of 125 μL containing 10² TCID₅₀ virus per well, and an additional 125 μL of MEM to the final lane (cell control). Virus and serum was then incubated for 1 h at room temperature and then 50 μL serum/virus mix to each well of 4 rows of Vero cells (quadruplicate). Five days post-infection CPE was assessed and the MN titre calculated by the Reed Muench method. LoD was defined as the reciprocal of the highest concentration of sera tested and any values falling below the LoD were reported as ½ LoD.

### Depletion MN Assay

The depletion MN assay was conducted as above with the following modifications; Sera were titrated in MEM in a round bottom plate and mixed 1:1 with the depletion antigen to give a final concentration of 10 μg/mL. Sera and depletion antigen were incubated at 37° C. for 1 hr. Following this, 100 TCID50 virus per well and was added as per MN assay and the virus, serum and depletion antigen incubated together for 1 h at RT before addition to the cell monolayer.

### Plaque Reduction Neutralization Assay (PRNT)

Two isolates made from infected patients in Queensland Australia were used for the PRNT assay, QLD02/2020-30/1/2020 (GISAID accession EPI_ISL_407896) and QLDID935/2020-25/03/2020 (GISAID accession EPI_ISL_436097). Viruses were passaged two times in Vero E6 cells and titrated by focus-forming assay on Vero E6 cells. Serial dilutions of the indicated sera and BALF were incubated with approximately 3000 focus-forming units of SARS-CoV-2 for 1 h at 37° C. Mab-virus complexes were added to Vero E6 cell monolayers at 37° C. for 30 min in 96-well plates that were pre-seeded at 40,000 cells/well and incubated overnight. Subsequently, cells were overlaid with 1% (w/v) medium velocity carboxymethyl cellulose in M199 (Gibco) supplemented with 2% heat-inactivated fetal bovine serum (HI-FCS) supplemented with 1% Penicillin-Streptomycin (Sigma-Aldrich) P/S. Plates were collected 14 h later by removing overlays and fixed with 80% cold-acetone in PBS for 1 h at −20° C. Plates were then dried and blocked with blocking buffer (1×KPL in 0.1% PBS-Tween 20) for 1 hour. Plates were subsequently incubated with 1 μg/mL of CR3022 anti-Spike antibody and 0.02 μg/mL IR-Dye800-conjugated goat anti-human IgG in blocking buffer. Plates were washed 3 times after antibody incubations by submerging in PBS-T 0.1% Tween-20. Plates were then dried prior to visualizing using Odyssey (LI-COR). Immunoplaques were manually counted in blinded fashion.

### FTA Analysis

The FTA analysis was performed using a well-established method as described in Mekonnen, Z. A. et al. (2019. J Virol 93: doi:10.1128/JVI.00202-19) and Grubor-Bauk, B. et al. (2019. Sci Adv 5(12):eaax2388). In brief, naïve autologous BALB/c splenocytes were evenly split and labeled serially with 0.12, 0.46, 1.7, 6.2, 23 or 85 μM of cell trace violet (CTV, Invitrogen) and or 39 μM cell proliferation dye (CPD, Invitrogen) for 5 min at RT. Each of the dye-labelled population was pulsed with DMSO (nil) or 10 μg/mL/peptide of the indicated peptide pools comprising of 15-18 aa peptides (10-11 aa overlap between adjacent peptides) for 4 h at 37° C. with 5% CO₂. Overlapping peptides spanning the SARS-CoV-2 S₁₋₁₂₂₆ and the Peptivator array (S₃₀₄₋₃₃₈, S₄₂₁₋₄₇₅, S₄₉₂₋₅₁₉, S₆₈₃₋₇₀₇, S₇₄₁₋₇₇₀, S₇₈₅₋₈₀₂ and S₈₈₅₋₁₂₇₃) used for peptide pulsing were purchased from Shanghai RoyoBiotech and Miltenyi Biotec, respectively. The FTA was then injected i.v. into placebo or vaccinated mice such that each mouse received 24×10⁶ cells (2×10⁶ cells from each fluorescent bar-coded target cell population) in 200 μL of PBS.

15 h after the injection, RBC-depleted splenocytes from FTA-challenged mice were stained with PE-Cy7 conjugated anti-mouse CD69 (clone H1.2F3, BD Biosciences) and BUV395 conjugated anti-mouse 8220 (clone RA3-662, BD Biosciences) and fixed using 0.5% paraformaldehyde. Subsequently, the stained samples were acquired using the BD LSRII and analyzed using the FlowJo software (version 8.8.7). The geometric mean fluorescent intensity (GMFI) of CD69 plotted was calculated using the formula: B220⁺ peptides-pulsed target value (GMFI of CD69)—B220⁺ nil target value (GMFI of CD69). The following formula was used to calculate the % killed data: [(nil target value %−peptides-pulsed target value %)/nil target value %]×100.

### ICS Analysis

2×10⁶ RBC-depleted splenocytes from vaccinated or placebo control mice were seeded in each well of a 96-well round-bottom plate and stimulated at 37° C. with 5% CO₂ for 12 h using 5 μg/mL of S1-1226 total peptide pool at a cell density of 1×10 7 cells/mL. DMSO and PMA/Ionomycin (PMA at 25 ng/mL and Ionomycin at 1 μg/mL) stimulations were included as negative and positive controls, respectively. Following the 12 h incubation, 1 μg/mL of brefeldin-A (BioLegend) was added to each well and incubated for further 4 h prior to staining the cells with fluorochrome conjugated monoclonal antibodies. The stimulated cells were stained for cell-surface markers, fixed and permeabilized using IC Fix/Perm buffer (BioLegend) prior to the intracellular stain to analyze cytokine expression. For the data presented, the following fluorochrome-conjugated monoclonal antibodies were used to stain the cells: CD3 (clone: 17A2, BioLegend), CD4 (clone: GK1.5, BioLegend), CD8 (clone: 53-6.7, BioLegend), IFN-γ (clone: XMG1.2, BioLegend), TNF-α (clone: MP6-XT22, BioLegend), IL-2 (clone: JES6-5H4, BioLegend), IL-4 (clone: 11611, BioLegend) and IL-13 (clone: eBio13A, eBioscience). The stained cells were acquired using the BD LSRII flow cytometer and analyzed using the FlowJo software (version 10.8).

### Statistical Significance Analysis

Statistical significance analysis of the data was performed using the IBM SPSS Statistics Software (version 25) or GraphPad Prism (version 8.3.1). P values are reported for statistically significant comparisons with p<0.05. Non-significant data with p>0.05 are denoted ‘ns’.

TABLE 9
Engineered Sclamp variants

| | | | Expression | Antibody |
|---|---|---|---|---|
| | | | (mg/L | Reactivity |
| Construct | Linker sequence | C-terminus | Transient | Cr3022 |
| Cycle 1 | (SEQ ID NOs 99 to 110) | (aa) | Purified) | Kd (nM) |
| S1GSG | 671-CASYQGSG------------SIIAY-695 | 1204 | 2.9 | 1.02 |
| S1GGSGG | 671-CASYQGGSGG----------SIIAY-695 | 1204 | 1.9 | 0.69 |
| S2GSG | 671-CASYQTQGSG--------SQSIIAY-695 | 1204 | 1.0 | 1.03 |
| S2GGSGG | 671-CASYQTQGGSGG------SQSIIAY-695 | 1204 | 2.4 | 1.18 |
| M1GSG | 671-CASYQTQTNGSG--------SIIAY-695 | 1204 | 4.6 | 0.58 |
| M1GGSGG | 671-CASYQTQTNGGSGG------SIIAY-695 | 1204 | 4.7 | 0.57 |
| W1GSG | 671-CASYQTQTNSPGSG-SVASQSIIAY-695 | 1204 | 1.6 | 1.08 |
| W1GGSG | 671-CASYQTQTNSPGGSGSVASQSIIAY-695 | 1204 | 1.6 | 0.89 |
| W2GSG | 671-CASYQTQTGSG-----VASQSIIAY-695 | 1204 | 2.1 | 2.06 |
| W2GGSGG | 671-CASYQTQTGGSGG---VASQSIIAY-695 | 1204 | 2.3 | 1.09 |
| RAA | 671-CASYQTQTNSPRRAASVASQSIIAY-695 | 1204 | 1.1 | 0.91 |
| AAAA | 671-CASYQTQTNSPAAAASVASQSIIAY-695 | 1204 | 1.3 | 0.74 |

| C-terminal truncation | | |
|---|---|---|
| | | C-terminus |
| | Linker sequence (SEQ ID NO: 111) | (aa) |
| M1GSG | 672-ASYQTQTNGSGS-691 | 1135-1210 |

| | | Modified | |
|---|---|---|---|
| | | SARS-CoV-2 | C- |
| | Signal Peptide Sequence | Spike | terminus |
| Origin | (SEQ ID NOs 112 to 118) | Protein | (aa) |
| SARS-CoV2 SS | MFVFLVLLPLVSSQCV | 672-ASYQTQ | 1204 |
| MERS SS VSS | MIHSVFLLMFLLTPTESVSSQCV | TNGSGS-691 | 1204 |
| MERS SS QCV | MIHSVFLLMFLLTPTESQCV | | 1204 |
| SARS-CoV1 SS | MFIFLLFLTLTSGVSSQCV | | 1204 |
| HKU SS | MFLIIFILPTTLAVSSQCV | | 1204 |
| Azur SS | MTRLTVLALLAGLLASSRAVSSQCV | | 1204 |

*Relative to wild-type SARS-CoV-2 with sequence: 672-ASYQTQTNSPRRARSVASQS-691 (SEQ ID NO: 86)

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the disclosure without limiting the disclosure to any one embodiment or specific collection of features. Those of skill in the art will therefore appreciate that, in light of the instant disclosure, various modifications and changes can be made in the particular embodiments exemplified without departing from the scope of the present disclosure. All such modifications and changes are intended to be included within the scope of the appended claims.

1. A modified SARS-CoV-2 spike polypeptide that is distinguished from a wild-type SARS-CoV-2 spike protein by an absence of a furin cleavage site at a location corresponding to the furin cleavage site of the wild-type SARS-CoV-2 spike protein and a presence of a heterologous flexible linker at the location.

2. The modified polypeptide of claim 1, wherein the flexible linker connects first and second polypeptides, wherein the first polypeptide corresponds to an upstream portion of the wild-type SARS-CoV-2 spike protein and the second polypeptide corresponds to a downstream portion of the wild-type SARS-CoV-2 spike protein,
wherein:
the carboxy-terminal residue of the upstream portion is immediately upstream of an amino acid corresponding to any one of Pro681, Ser680, Asn679, Thr678, Gln677, and Thr676, and the amino-terminal residue of the downstream portion is immediately downstream of an amino acid corresponding to any one of Ser686, Val687, Ala688, Ser689, Gln690 and Ser691 of the full-length wild-type SARS-CoV-2 spike protein amino acid sequence set forth in SEQ ID NO:1; and/or
the carboxy-terminal residue of the upstream portion is immediately downstream of an amino acid corresponding to any one of Gln675, Thr676, Gln677, Thr678, Asn679, and Ser680, and the amino-terminal residue of the downstream portion is immediately upstream of an amino acid corresponding to any one of Ser691, Gln690, Ser689, Ala688, Val687, and Ser686 of the full-length wild-type SARS-CoV-2 spike protein amino acid sequence set forth in SEQ ID NO:1.

3. The modified polypeptide of claim 2, wherein the carboxy-terminal residue of the upstream portion corresponds to an amino acid residue selected from Pro681, Ser680, Asn679, Thr678, Gln677, Thr676 and Gln675, and the amino-terminal residue of the downstream portion corresponds to an amino acid residue selected from Ser686, Val687, Ala688, Ser689, Gln690 and Ser691.

4-5. (canceled)

6. The modified polypeptide of claim 1, wherein the modified polypeptide lacks an amino acid residue corresponding to Pro681 and/or Ala684 of the SARS-CoV-2 spike protein amino acid sequence set forth in SEQ ID NO:1.

7. (canceled)

8. The modified polypeptide of claim 1, wherein the upstream and downstream portions of the wild-type SARS-CoV-2 spike protein comprise an amino acid sequence having at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98% or 99% sequence similarity or identity to the amino acid sequence set forth in SEQ ID NO:1.

9. The modified polypeptide of claim 1, wherein the first and second polypeptides of the modified polypeptide have at least 75%, 76%, 77%, 78%, 79% 80%, 81%, 82%, 83%, 84%, 85%, 86%, 87%, 88%, 89%, 90%, 91%, 92%, 93%, 94%, 95%, 96%, 97%, 98% or 99% sequence similarity or identity to the amino acid sequence set forth in SEQ ID NO:1.

10. The modified polypeptide of claim 1, wherein the flexible linker consists or consists essentially of glycine and serine residues.

11-15. (canceled)

16. The modified polypeptide of claim 1, wherein the modified polypeptide is operably connected downstream to a heterologous structure-stabilizing moiety.

17. The modified polypeptide of claim 16, wherein the structure-stabilizing moiety stabilizes the modified polypeptide in a conformation that mimics the pre-fusion conformation of the wild-type SARS-CoV-2 spike protein.

18. (canceled)

19. The modified polypeptide of claim 16, wherein the structure-stabilizing moiety comprises a trimerization domain.

20. (canceled)

21. The modified polypeptide of claim 19, wherein the trimerization domain comprises complementary first heptad repeat (HR1) and second heptad repeat (HR2) regions that associate with each other under conditions suitable for their association to form an anti-parallel, two-helix bundle.

22. (canceled)

23. The modified polypeptide of claim 21, wherein
each of the HR1 and HR2 regions is independently characterized by an n-times repeated 7-residue pattern of amino acid types, represented as (a-b-c-d-e-f-g-)ₙ or (d-e-f-g-a-b-c)ₙ, wherein the pattern elements ‘a’ to ‘g’ denote heptad positions at which the amino acid types are located and n is a number equal to or greater than 2, and at least 50% of the heptad positions ‘a’ and ‘d’ are occupied by hydrophobic amino acid types and at least 50% of the heptad positions ‘b’, ‘c’, ‘e’, ‘f’ and ‘g’ are occupied by hydrophilic amino acid types, the resulting distribution between hydrophobic and hydrophilic amino acid types enabling the identification of the heptad repeat regions.

24. The modified polypeptide of claim 21, wherein one or both of the HR1 and HR2 regions comprises an endogenous Class I enveloped virus fusion protein heptad repeat region amino acid sequence.

25. The modified polypeptide of claim 24, wherein the HR1 and HR2 regions comprise complementary endogenous heptad repeat A (HRA) and heptad repeat B (HRB) regions, respectively, of one or more Class I enveloped virus fusion proteins.

26.-27. (canceled)

28. The modified polypeptide of claim 25, wherein the HR1 and HR2 regions are independently selected from HRA and HRB regions of fusion proteins expressed by orthomyxoviruses, paramyxoviruses, retroviruses, coronaviruses, filoviruses and arenaviruses.

29. The modified polypeptide of claim 16, wherein the structure-stabilizing moiety is operably connected directly or indirectly to the carboxy-terminal residue of an amino acid sequence corresponding to the SARS-CoV-2 ectodomain polypeptide.

30. (canceled)

31. The modified polypeptide of claim 29, wherein the carboxy-terminal residue corresponds to Gly1204 of the SARS-CoV-2 spike protein.

32.-34. (canceled)

35. The modified polypeptide of claim 16, wherein the structure-stabilizing moiety, including one or both of the heptad repeat regions, includes an immune-silencing or suppressing moiety that inhibits elicitation or production of an immune response to the structure-stabilizing moiety.

36. The modified polypeptide of claim 35, wherein the immune-silencing moiety comprises a glycosylation site that is specifically recognized and glycosylated by a glycosylation enzyme.

37. A polynucleotide comprising a coding sequence for a modified polypeptide, as defined in claim 1.

38. The polynucleotide of claim 37, wherein the modified polypeptide is a modified polypeptide, as defined in claim 16.

39. The polynucleotide of claim 38, wherein the polynucleotide comprises RNA.

40. A nucleic acid construct that comprises a polynucleotide as defined in claim 38, operably linked to a regulatory element that is operable in a host cell.

41. A host cell that contains a nucleic acid construct as defined in claim 40.

42.-43. (canceled)

44. A method of producing a polypeptide complex, the method comprising: combining modified polypeptides as defined in claim 1 under conditions suitable for the formation of a polypeptide complex, whereby a polypeptide complex is produced that comprises three modified polypeptides.

45. The method of claim 44, wherein the modified polypeptides are as defined in claim 16.

46. (canceled)

47. A polypeptide complex comprising a trimer of modified polypeptides as defined in claim 1.

48. The polypeptide complex of claim 47, wherein the trimer of modified polypeptides is a trimer of modified polypeptides as defined in claim 16.

49.-51. (canceled)

52. The method of claim 68, wherein the modified polypeptide is a modified polypeptide as defined in claim 16.

53. The method of claim 52, wherein the ACE2-interacting coronavirus is SARS-CoV-2.

54. (canceled)

55. The method of claim 69, wherein the modified polypeptide is a modified polypeptide as defined in claim 16.

56.-60. (canceled)

61. A method for treating or inhibiting the development of an ACE2-interacting coronavirus infection, or at least one symptom, or viral shedding, associated therewith, in a subject, wherein the method comprises administering to the subject an effective amount of a modified polypeptide as defined in claim 16.

62. The method of claim 61, wherein the ACE2-interacting coronavirus is SARS-CoV-2.

63. A method for treating or inhibiting the development of COVID-19, or at least one symptom associated therewith, or reducing viral shedding associated with COVID-19, in a subject, wherein the method comprises administering to the subject an effective amount of a modified polypeptide as defined in claim 16.

64.-67. (canceled)

68. A method of eliciting an immune response to the spike protein of an ACE2-interacting coronavirus, or complex thereof, in a subject, wherein the method comprises administering to the subject an effective amount of a modified polypeptide as defined in claim 1.

69. A method of producing an antigen-binding molecule that is immuno-interactive with a SARS-CoV-2 spike protein, or complex thereof, the method comprising: (1) screening a library of antigen-binding molecules with a modified polypeptide as defined in claim 1; (2) detecting an antigen-binding molecule that binds specifically with the modified polypeptide; and (3) isolating the detected antigen-binding molecule.

70. A method for treating or inhibiting the development of an ACE2-interacting coronavirus infection, or at least one symptom, or viral shedding, associated therewith, in a subject, wherein the method comprises administering to the subject an effective amount of the modified polypeptide of claim 1.

71. A method for treating or inhibiting the development of an ACE2-interacting coronavirus infection, or at least one symptom, or viral shedding, associated therewith, in a subject, wherein the method comprises administering to the subject an effective amount of the polynucleotide of claim 38.


SEQUENCE LISTING (118 sequences)

SEQ ID NO: 1 - protein, 1273 aa, SARS-CoV-2
  full-length wild-type SARS-CoV-2 spike protein amino acid sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 5 aa, SARS-CoV-2
  wild-type SARS-CoV-2 spike protein amino acid sequence
PRRAR

SEQ ID NO: 3 - protein, 6 aa, SARS-CoV-2
  wild-type SARS-CoV-2 spike protein amino acid sequence
PRRARS

SEQ ID NO: 4 - protein, 7 aa, SARS-CoV-2
  wild-type SARS-CoV-2 spike protein amino acid sequence
PRRARSV

SEQ ID NO: 5 - protein, 8 aa, SARS-CoV-2
  wild-type SARS-CoV-2 spike protein amino acid sequence
PRRARSVA

SEQ ID NO: 6 - protein, 9 aa, SARS-CoV-2
  wild-type SARS-CoV-2 spike protein amino acid sequence
PRRARSVAS

SEQ ID NO: 7 - protein, 10 aa, SARS-CoV-2
  wild-type SARS-CoV-2 spike protein amino acid sequence
PRRARSVASQ

SEQ ID NO: 8 - protein, 6 aa, SARS-CoV-2
  wild-type SARS-CoV-2 spike protein amino acid sequence
SPRRAR

SEQ ID NO: 9 - protein, 7 aa, SARS-CoV-2
  wild-type SARS-CoV-2 spike protein amino acid sequence
SPRRARS

SEQ ID NO: 10 - protein, 8 aa, SARS-CoV-2
  wild-type SARS-CoV-2 spike protein amino acid sequence
SPRRARSV

SEQ ID NO: 11 - protein, 9 aa, SARS-CoV-2
  wild-type SARS-CoV-2 spike protein amino acid sequence
SPRRARSVA

SEQ ID NO: 12 - protein, 10 aa, SARS-CoV-2
  wild-type SARS-CoV-2 spike protein amino acid sequence
SPRRARSVAS

SEQ ID NO: 13 - protein, 11 aa, SARS-CoV-2
  wild-type SARS-CoV-2 spike protein amino acid sequence
SPRRARSVASQ

SEQ ID NO: 14 - protein, 7 aa, SARS-CoV-2
  wild-type SARS-CoV-2 spike protein amino acid sequence
NSPRRAR

SEQ ID NO: 15 - protein, 8 aa, SARS-CoV-2
  wild-type SARS-CoV-2 spike protein amino acid sequence
NSPRRARS

SEQ ID NO: 16 - protein, 9 aa, SARS-CoV-2
  wild-type SARS-CoV-2 spike protein amino acid sequence
NSPRRARSV

SEQ ID NO: 17 - protein, 10 aa, SARS-CoV-2
  wild-type SARS-CoV-2 spike protein amino acid sequence
NSPRRARSVA

SEQ ID NO: 18 - protein, 11 aa, SARS-CoV-2
  wild-type SARS-CoV-2 spike protein amino acid sequence
NSPRRARSVAS

SEQ ID NO: 19 - protein, 12 aa, SARS-CoV-2
  wild-type SARS-CoV-2 spike protein amino acid sequence
NSPRRARSVASQ

SEQ ID NO: 20 - protein, 8 aa, SARS-CoV-2
  wild-type SARS-CoV-2 spike protein amino acid sequence
TNSPRRAR

SEQ ID NO: 21 - protein, 9 aa, SARS-CoV-2
  wild-type SARS-CoV-2 spike protein amino acid sequence
TNSPRRARS

SEQ ID NO: 22 - protein, 10 aa, SARS-CoV-2
  wild-type SARS-CoV-2 spike protein amino acid sequence
TNSPRRARSV

SEQ ID NO: 23 - protein, 11 aa, SARS-CoV-2
  wild-type SARS-CoV-2 spike protein amino acid sequence
TNSPRRARSVA

SEQ ID NO: 24 - protein, 12 aa, SARS-CoV-2
  wild-type SARS-CoV-2 spike protein amino acid sequence
TNSPRRARSVAS

SEQ ID NO: 25 - protein, 13 aa, SARS-CoV-2
  wild-type SARS-CoV-2 spike protein amino acid sequence
TNSPRRARSVASQ

SEQ ID NO: 26 - protein, 9 aa, SARS-CoV-2
  wild-type SARS-CoV-2 spike protein amino acid sequence
QTNSPRRAR

SEQ ID NO: 27 - protein, 10 aa, SARS-CoV-2
  wild-type SARS-CoV-2 spike protein amino acid sequence
QTNSPRRARS

SEQ ID NO: 28 - protein, 11 aa, SARS-CoV-2
  wild-type SARS-CoV-2 spike protein amino acid sequence
QTNSPRRARSV

SEQ ID NO: 29 - protein, 12 aa, SARS-CoV-2
  wild-type SARS-CoV-2 spike protein amino acid sequence
QTNSPRRARSVA

SEQ ID NO: 30 - protein, 13 aa, SARS-CoV-2
  wild-type SARS-CoV-2 spike protein amino acid sequence
QTNSPRRARSVAS

SEQ ID NO: 31 - protein, 14 aa, SARS-CoV-2
  wild-type SARS-CoV-2 spike protein amino acid sequence
QTNSPRRARSVASQ

SEQ ID NO: 32 - protein, 10 aa, SARS-CoV-2
  wild-type SARS-CoV-2 spike protein amino acid sequence
TQTNSPRRAR

SEQ ID NO: 33 - protein, 11 aa, SARS-CoV-2
  wild-type SARS-CoV-2 spike protein amino acid sequence
TQTNSPRRARS

SEQ ID NO: 34 - protein, 12 aa, SARS-CoV-2
  wild-type SARS-CoV-2 spike protein amino acid sequence
TQTNSPRRARSV

SEQ ID NO: 35 - protein, 13 aa, SARS-CoV-2
  wild-type SARS-CoV-2 spike protein amino acid sequence
TQTNSPRRARSVA

SEQ ID NO: 36 - protein, 14 aa, SARS-CoV-2
  wild-type SARS-CoV-2 spike protein amino acid sequence
TQTNSPRRARSVAS

SEQ ID NO: 37 - protein, 15 aa, SARS-CoV-2
  wild-type SARS-CoV-2 spike protein amino acid sequence
TQTNSPRRARSVASQ

SEQ ID NO: 38 - protein, 27 aa, bacteriophage T4
  trimerization domain (foldon) of the bacteriophage T4 protein fibritin
GYIPEAPRDGQAYVRKDGEWVLLSTFL

SEQ ID NO: 39 - protein, 29 aa, bacteriophage T4
  trimerization domain (foldon) of the bacteriophage T4 protein fibritin
GSGYIPEAPRDGQAYVRKDGEWVLLSTFL

SEQ ID NO: 40 - protein, 32 aa, Saccharomyces cerevisiae
  GCN4 leucine zipper domain capable of forming a trimer
RMKQIEDKIEEILSKIYHIENEIARIKKLIGE

SEQ ID NO: 41 - protein, 27 aa, Saccharomyces cerevisiae
  GCN4 leucine zipper domain capable of forming a trimer
MKQIEDKIEEIESKQKKIENEIARIKK

SEQ ID NO: 42 - protein, 25 aa, Artificial Sequence
  HR1 and HR2 region
IEEIQKQIAAIQKQIAAIQKQIYRM

SEQ ID NO: 43 - protein, 37 aa, Human immunodeficiency virus
  HRA region amino acid sequence
SGIVQQQNNLLRAIEAQQHLLQLTVWGIKQLQARILA

SEQ ID NO: 44 - protein, 39 aa, Respiratory syncytial virus
  HRA region amino acid sequence
LHLEGEVNKIKSALLSTNKAVVSLGNGVSVLTSKVLDLK

SEQ ID NO: 45 - protein, 39 aa, Human metapneumovirus
  HRA region amion acid sequence
IRLESEVTAIKNALKKTNEAVSTLGNGVRVLATAVRELK

SEQ ID NO: 46 - protein, 39 aa, Parainfluenza virus
  HRA region amino acid sequence
KQARSDIEKLKEAIRDTNKAVQSVQSSIGNLIVAIKSVQ

SEQ ID NO: 47 - protein, 39 aa, Measles virus
  HRA region amino acid sequence
MLNSQAIDNLRASLETTNQAIEAIRQAGQEMILAVQGVQ

SEQ ID NO: 48 - protein, 39 aa, Hendra virus
  HRA region amino acid sequence
MKNADNINKLKSSIESTNEAVVKLQETAEKTVYVLTALQ

SEQ ID NO: 49 - protein, 35 aa, Influenza A virus
  HRA region amino acid sequence
ENGWEGMVDGWYGFRHQNSEGTGQAADLKSTQAAI

SEQ ID NO: 50 - protein, 34 aa, Influenza B virus
  HRA region amino acid sequence
HGYTSHGAHGVAVAADLKSTQEAINKITKNLNYL

SEQ ID NO: 51 - protein, 37 aa, Ebola virus sp.
  HRA region amino acid sequence
GLRQLANETTQALQLFLRATTELRTFSILNRKAIDFL

SEQ ID NO: 52 - protein, 38 aa, Marburg virus
  HRA region amino acid sequence
LANQTAKSLELLLRVTTEERTFSLINRHAIDFLLTRWG

SEQ ID NO: 53 - protein, 59 aa, Middle East Respiratory Syndrome virus
  HRA region amino acid sequence
ISASIGDIIQRLDVLEQDAQIDRLINGRLTTLNAFVAQQLVRSESAALSAQLAKDKVNE

SEQ ID NO: 54 - protein, 59 aa, Severe acute respiratory syndrome virus
  HRA region amino acid sequence
ISSVLNDILSRLDKVEAEVQIDRLITGRLQSLQTYVTQQLIRAAEIRASANLAATKMSE

SEQ ID NO: 55 - protein, 38 aa, Human immunodeficiency virus
  HRB region amino acid sequence
HTTWMEWDREINNYTSLIHSLIEESQNQQEKNEQELLE

SEQ ID NO: 56 - protein, 37 aa, Respiratory syncytial virus
  HRB region amino acid sequence
FDASISQVNEKINQSLAFIRKSDELLHNVNAGKSTTN

SEQ ID NO: 57 - protein, 35 aa, Human metapneumovirus
  HRB region amino acid sequence
FNVALDQVFESIENSQALVDQSNRILSSAEKGNTG

SEQ ID NO: 58 - protein, 36 aa, Parainfluenza virus
  HRB region amino acid sequence
IELNKAKSDLEESKEWIRRSNQKLDSIGNWHQSSTT

SEQ ID NO: 59 - protein, 40 aa, Measles virus
  HRB region amino acid sequence
LERLDVGTNLGNAIAKLEDAKELLESSDQILRSMKGLSST

SEQ ID NO: 60 - protein, 32 aa, Hendra virus
  HRB region amino acid sequence
ISSQISSMNQSLQQSKDYIKEAQKILDTVNPS

SEQ ID NO: 61 - protein, 49 aa, Influenza A virus
  HRB region amino acid sequence
RIQDLEKYVEDTKIDLWSYNAELVLALENQHTIDLTDSEMSKLFERTRR

SEQ ID NO: 62 - protein, 52 aa, Influenza B virus
  HRB region amino acid sequence
DEILELDEKVDDLRADTISSQIELAVLLSNEGIINSEDEHLLALERKLKKML

SEQ ID NO: 63 - protein, 23 aa, Ebola virus sp.
  HRB region amino acid sequence
HDWTKNITDKIDQIIHDFVDKTL

SEQ ID NO: 64 - protein, 17 aa, Marburg virus
  HRB region amino acid sequence
IGIEDLSKNISEQIDQI

SEQ ID NO: 65 - protein, 44 aa, Middle East Respiratory Syndrome virus
  HRB region amino acid sequence
NFGSLTQINTTLLDLTYEMLSLQQVVKALNESYIDLKELGNYTY

SEQ ID NO: 66 - protein, 43 aa, Severe acute respiratory syndrome virus
  HRB region amino acid sequence
DVDLGDISGINASVVNIQKEIDRLNEVAKNLNESLIDLQELGK

SEQ ID NO: 67 - protein, 1272 aa, Artificial Sequence
  SARS-CoV-2 Spike protein- Q675-GSG-S691 G1204-HIV GP160-based SSM
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQGSGSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGIVQQQNNLLRAIEAQQHLLQLTVWGIKQLQARILAGGSGGHTTWMEWDREINNYTSLIHSLIEESQNQQEKNEQELLE

SEQ ID NO: 68 - protein, 1274 aa, Artificial Sequence
  SARS-CoV-2 Spike protein - Q675-GGSGG-S691 G1204-HIV GP160-based SSM
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQGGSGGSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGIVQQQNNLLRAIEAQQHLLQLTVWGIKQLQARILAGGSGGHTTWMEWDREINNYTSLIHSLIEESQNQQEKNEQELLE

SEQ ID NO: 69 - protein, 1276 aa, Artificial Sequence
  SARS-CoV-2 Spike protein - Q677-GSG-S689 G1204-HIV GP160-based SSM
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQGSGSQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGIVQQQNNLLRAIEAQQHLLQLTVWGIKQLQARILAGGSGGHTTWMEWDREINNYTSLIHSLIEESQNQQEKNEQELLE

SEQ ID NO: 70 - protein, 1278 aa, Artificial Sequence
  SARS-CoV-2 Spike protein - Q677-GGSGG-S689 G1204-HIV GP160-based SSM
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQGGSGGSQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGIVQQQNNLLRAIEAQQHLLQLTVWGIKQLQARILAGGSGGHTTWMEWDREINNYTSLIHSLIEESQNQQEKNEQELLE

SEQ ID NO: 71 - protein, 1276 aa, Artificial Sequence
  SARS-CoV-2 Spike protein - N679-GSG-S691 G1204-HIV GP160-based SSM
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNGSGSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGIVQQQNNLLRAIEAQQHLLQLTVWGIKQLQARILAGGSGGHTTWMEWDREINNYTSLIHSLIEESQNQQEKNEQELLE

SEQ ID NO: 72 - protein, 1278 aa, Artificial Sequence
  SARS-CoV-2 Spike protein - N679-GGSGG-S691 G1204-HIV GP160-based SSM
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNGGSGGSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGIVQQQNNLLRAIEAQQHLLQLTVWGIKQLQARILAGGSGGHTTWMEWDREINNYTSLIHSLIEESQNQQEKNEQELLE

SEQ ID NO: 73 - protein, 1283 aa, Artificial Sequence
  SARS-CoV-2 Spike protein - P681-GSG-S686 G1204-HIV GP160-based SSM
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGIVQQQNNLLRAIEAQQHLLQLTVWGIKQLQARILAGGSGGHTTWMEWDREINNYTSLIHSLIEESQNQQEKNEQELLE

SEQ ID NO: 74 - protein, 1285 aa, Artificial Sequence
  SARS-CoV-2 Spike protein - P681-GGSGG-S691 G1204-HIV GP160-based SSM
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGSGGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGIVQQQNNLLRAIEAQQHLLQLTVWGIKQLQARILAGGSGGHTTWMEWDREINNYTSLIHSLIEESQNQQEKNEQELLE

SEQ ID NO: 75 - protein, 1277 aa, Artificial Sequence
  SARS-CoV-2 Spike protein - N679-GSG-S691 G1204-Immunosilenced HIV GP160-based SSM
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNGSGSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGIVQQQNNLLRAIEAQQHLLQLTVWGIKQLQARILAGGSGGNHTTWMNWSREINNYTSLIHNLTEESQNQTEKNEQELLE

SEQ ID NO: 76 - protein, 4 aa, Artificial Sequence
  linker sequence
GGSG

SEQ ID NO: 77 - protein, 5 aa, Artificial Sequence
  linker sequence
GGSGG

SEQ ID NO: 78 - protein, 6 aa, Artificial Sequence
  linker sequence
GGSGGG

SEQ ID NO: 79 - protein, 5 aa, Artificial Sequence
  linker sequence
GGGGS

SEQ ID NO: 80 - protein, 4 aa, Artificial Sequence
  linker sequence
SSSG

SEQ ID NO: 81 - protein, 5 aa, Artificial Sequence
  linker sequence
SSSSG

SEQ ID NO: 82 - protein, 4 aa, Artificial Sequence
  linker sequence
AAPA

SEQ ID NO: 83 - protein, 8 aa, Artificial Sequence
  linker sequence
GGGKGGGG

SEQ ID NO: 84 - protein, 8 aa, Artificial Sequence
  linker sequence
GGGNGGGG

SEQ ID NO: 85 - protein, 8 aa, Artificial Sequence
  linker sequence
GGGCGGGG

SEQ ID NO: 86 - protein, 20 aa, Artificial Sequence
  wild-type SARS-CoV-2 Spike Protein
ASYQTQTNSPRRARSVASQS

SEQ ID NO: 87 - protein, 1273 aa, Artificial Sequence
  primary sequence of SARS-CoV-2 Sclamp-RSV
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNGSGSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGVNKIKSALLSTNKAVVSLSNGVSVLTSKVLDLKGGSGGFDASISQVNEKINQSLAFIRKSDELLHNVNAGKSTTN

SEQ ID NO: 88 - protein, 1282 aa, Artificial Sequence
  primary sequence of SARS-CoV-2 Sclamp-hMPV F
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNGSGSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGKTIRLESEVTAIKNALKKTNEAVSTLGNGVRVLATAVRELKDFVGGSGGFNVALDQVFESIENSQALVDQSNRILSSAEKGNTG

SEQ ID NO: 89 - protein, 1272 aa, Artificial Sequence
  primary sequence of SARS-CoV-2 Sclamp-PIV
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNGSGSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGEAKQARSDIEKLKEAIRDTNKAVQSVQSSIGNLGGSGGIELNKAKSDLEESKEWIRRSNQKLDSIGNWHQSSTT

SEQ ID NO: 90 - protein, 1277 aa, Artificial Sequence
  Primary sequence of SARS-CoV-2 Sclamp-MEV F
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNGSGSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGHQSMLNSQAIDNLRASLETTNQAIEAIRQAGQEMGGSGGLERLDVGTNLGNAIAKLEDAKELLESSDQILRSMKGLSST

SEQ ID NO: 91 - protein, 1269 aa, Artificial Sequence
  primary sequence of SARS-CoV-2 Sclamp-HEV F
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNGSGSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGEAMKNADNINKLKSSIESTNEAVVKLQETAEKTVGGSGGISSQISSMNQSLQQSKDYIKEAQKILDTVNPS

SEQ ID NO: 92 - protein, 1287 aa, Artificial Sequence
  primary sequence of SARS-CoV-2 Sclamp-INF A HA
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNGSGSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGENGWEGMVDGWYGFRHQNSEGTGQAADLKSTQAAIGGSGGRIQDLEKYVEDTKIDLWSYNAELLVALENQHTIDLTDSEMSKLFERTRR

SEQ ID NO: 93 - protein, 1289 aa, Artificial Sequence
  primary sequence of SARS-CoV-2 Sclamp-INF B
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNGSGSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGHGYTSHGAHGVAVAADLKSTQEAINKITKNLNYLGGSGGDEILELDEKVDDLRADTISSQIELAVLLSNEGIINSEDEHLLALERKLKKML

SEQ ID NO: 94 - protein, 1276 aa, Artificial Sequence
  primary sequence of SARS-CoV-2 Sclamp-EBOV GP
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNGSGSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGGLRQLANETTQALQLFLRATTELRTFSILNRKAIDFLGGSGGTCHILGPDCCIEPHDWTKNITDKIDQIIHDFVDKTL

SEQ ID NO: 95 - protein, 1238 aa, Artificial Sequence
  primary sequence of SARS-CoV-2 Sclamp-MARV GP
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNGSGSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGTFSLINRHAIDFLLTRWGGSGGIGIEDLSKNISEQIDQIK

SEQ ID NO: 96 - protein, 1369 aa, Artificial Sequence
  primary sequence of SARS-CoV-2 Sclamp-MERS S
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNGSGSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGGITQQVLSENQKLIANKFNQALGAMQTGFTTTNEAFRKVQDAVNNNAQALSKLASELSNTFGAISASIGDIIQRLDVLEQDAQIDRLINGRLTTLNAFVAQQLVRSESAALSAQLAKDKVNEGGSGGNFGSLTQINTTLLDLTYEMLSLQQVVKALNESYIDLKELGNYTY

SEQ ID NO: 97 - protein, 1368 aa, Artificial Sequence
  primary sequence of SARS-CoV-2 Sclamp-SARS
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNGSGSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSEGGSGGDVDLGDISGINASVVNIQKEIDRLNEVAKNLNESLIDLQELGK

SEQ ID NO: 98 - protein, 1230 aa, Artificial Sequence
  primary sequence of the SARS-CoV-2-foldon
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNGSGSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGSGYIPEAPRDGQAYVRKDGEWVLLSTFGSGEPEA

SEQ ID NO: 99 - protein, 13 aa, Artificial Sequence
  linker sequence
CASYQGSGSIIAY

SEQ ID NO: 100 - protein, 15 aa, Artificial Sequence
  linker sequence
CASYQGGSGGSIIAY

SEQ ID NO: 101 - protein, 17 aa, Artificial Sequence
  linker sequence
CASYQTQGSGSQSIIAY

SEQ ID NO: 102 - protein, 19 aa, Artificial Sequence
  linker sequence
CASYQTQGGSGGSQSIIAY

SEQ ID NO: 103 - protein, 17 aa, Artificial Sequence
  linker sequence
CASYQTQTNGSGSIIAY

SEQ ID NO: 104 - protein, 19 aa, Artificial Sequence
  linker sequence
CASYQTQTNGGSGGSIIAY

SEQ ID NO: 105 - protein, 24 aa, Artificial Sequence
  linker sequence
CASYQTQTNSPGSGSVASQSIIAY

SEQ ID NO: 106 - protein, 25 aa, Artificial Sequence
  linker sequence
CASYQTQTNSPGGSGSVASQSIIAY

SEQ ID NO: 107 - protein, 20 aa, Artificial Sequence
  linker sequence
CASYQTQTGSGVASQSIIAY

SEQ ID NO: 108 - protein, 22 aa, Artificial Sequence
  linker sequence
CASYQTQTGGSGGVASQSIIAY

SEQ ID NO: 109 - protein, 25 aa, Artificial Sequence
  linker sequence
CASYQTQTNSPRRAASVASQSIIAY

SEQ ID NO: 110 - protein, 25 aa, Artificial Sequence
  linker sequence
CASYQTQTNSPAAAASVASQSIIAY

SEQ ID NO: 111 - protein, 12 aa, Artificial Sequence
  linker sequence
ASYQTQTNGSGS

SEQ ID NO: 112 - protein, 16 aa, Artificial Sequence
  Signal Peptide Sequence
MFVFLVLLPLVSSQCV

SEQ ID NO: 113 - protein, 23 aa, Artificial Sequence
  Signal Peptide Sequence
MIHSVFLLMFLLTPTESVSSQCV

SEQ ID NO: 114 - protein, 20 aa, Artificial Sequence
  Signal Peptide Sequence
MIHSVFLLMFLLTPTESQCV

SEQ ID NO: 115 - protein, 19 aa, Artificial Sequence
  Signal Peptide Sequence
MFIFLLFLTLTSGVSSQCV

SEQ ID NO: 116 - protein, 19 aa, Artificial Sequence
  Signal Peptide Sequence
MFLIIFILPTTLAVSSQCV

SEQ ID NO: 117 - protein, 25 aa, Artificial Sequence
  Signal Peptide Sequence
MTRLTVLALLAGLLASSRAVSSQCV

SEQ ID NO: 118 - protein, 12 aa, Artificial Sequence
  Modified SARS-CoV-2 Spike Protein
ASYQTQTNGSGS